SEQUENCE LISTING (109 sequences)

SEQ ID NO: 1 - DNA, 1522 bp, Homo sapiens
  CDS (13)..(1389)
  mat_peptide (124)..(1389)
acagtcagccgcatggctcccctgtgccccagcccctggctccctctgttgatcccggcccctgctccaggcctcactgtgcaactgctgctgtcactgctgcttctgatgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataat

SEQ ID NO: 2 - protein, 459 aa, Homo sapiens
MAPLCPSPWLPLLIPAPAPGLTVQLLLSLLLLMPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 3 - DNA, 10898 bp, Homo sapiens
  gene (1)..(10898)
  misc_feature (1974)..(1974): n is a, c, g, or t
ggatcctgttgactcgtgaccttacccccaaccctgtgctctctgaaacatgagctgtgtccactcagggttaaatggattaagggcggtgcaagatgtgctttgttaaacagatgcttgaaggcagcatgctcgttaagagtcatcaccaatccctaatctcaagtaatcagggacacaaacactgcggaaggccgcagggtcctctgcctaggaaaaccagagacctttgttcacttgtttatctgaccttccctccactattgtccatgaccctgccaaatccccctctgtgagaaacacccaagaattatcaataaaaaaataaatttaaaaaaaaaatacaaaaaaaaaaaaaaaaaaaaaaaaagacttacgaatagttattgataaatgaatagctattggtaaagccaagtaaatgatcatattcaaaaccagacggccatcatcacagctcaagtctacctgatttgatctctttatcattgtcattctttggattcactagattagtcatcatcctcaaaattctcccccaagttctaattacgttccaaacatttaggggttacatgaagcttgaacctactaccttctttgcttttgagccatgagttgtaggaatgatgagtttacaccttacatgctggggattaatttaaactttacctctaagtcagttgggtagcctttggcttatttttgtagctaattttgtagttaatggatgcactgtgaatcttgctatgatagttttcctccacactttgccactaggggtaggtaggtactcagttttcagtaattgcttacctaagaccctaagccctatttctcttgtactggcctttatctgtaatatgggcatatttaatacaatataatttttggagtttttttgtttgtttgtttgtttgtttttttgagacggagtcttgcatctgtcatgcccaggctggagtagcagtggtgccatctcggctcactgcaagctccacctcccgagttcacgccattttcctgcctcagcctcccgagtagctgggactacaggcgcccgccaccatgcccggctaattttttgtatttttggtagagacggggtttcaccgtgttagccagaatggtctcgatctcctgacttcgtgatccacccgcctcggcctcccaaagttctgggattacaggtgtgagccaccgcacctggccaattttttgagtcttttaaagtaaaaatatgtcttgtaagctggtaactatggtacatttccttttattaatgtggtgctgacggtcatataggttcttttgagtttggcatgcatatgctactttttgcagtcctttcattacatttttctctcttcatttgaagagcatgttatatcttttagcttcacttggcttaaaaggttctctcattagcctaacacagtgtcattgttggtaccacttggatcataagtggaaaaacagtcaagaaattgcacagtaatacttgtttgtaagagggatgattcaggtgaatctgacactaagaaactcccctacctgaggtctgagattcctctgacattgctgtatataggcttttcctttgacagcctgtgactgcggactatttttcttaagcaagatatgctaaagttttgtgagcctttttccagagagaggtctcatatctgcatcaagtgagaacatataatgtctgcatgtttccatatttcaggaatgtttgcttgtgttttatgcttttatatagacagggaaacttgttcctcagtgacccaaaagaggtgggaattgttattggatatcatcattggcccacgctttctgaccttggaaacaattaagggttcataatctcaattctgtcagaattggtacaagaaatagctgctatgtttcttgacattccacttggtaggaaataagaatgtgaaactcttcagttggtgtgtgtccctngtttttttgcaatttccttcttactgtgttaaaaaaaagtatgatcttgctctgagaggtgaggcattcttaatcatgatctttaaagatcaataatataatcctttcaaggattatgtctttattataataaagataatttgtctttaacagaatcaataatataatcccttaaaggattatatctttgctgggcgcagtggctcacacctgtaatcccagcactttgggtggccaaggtggaaggatcaaatttgcctacttctatattatcttctaaagcagaattcatctctcttccctcaatatgatgatattgacagggtttgccctcactcactagattgtgagctcctgctcagggcaggtagcgttttttgtttttgtttttgtttttcttttttgagacagggtcttgctctgtcacccaggccagagtgcaatggtacagtctcagctcactgcagcctcaaccgcctcggctcaaaccatcatcccatttcagcctcctgagtagctgggactacaggcacatgccattacacctggctaatttttttgtatttctagtagagacagggtttggccatgttgcccgggctggtctcgaactcctggactcaagcaatccacccacctcagcctcccaaaatgagggaccgtgtcttattcatttccatgtccctagtccatagcccagtgctggacctatggtagtactaaataaatatttgttgaatgcaatagtaaatagcatttcagggagcaagaactagattaacaaaggtggtaaaaggtttggagaaaaaaataatagtttaatttggctagagtatgagggagagtagtaggagacaagatggaaaggtctcttgggcaaggttttgaaggaagttggaagtcagaagtacacaatgtgcatatcgtggcaggcagtggggagccaatgaaggcttttgagcaggagagtaatgtgttgaaaaataaatataggttaaacctatcagagcccctctgacacatacacttgcttttcattcaagctcaagtttgtctcccacatacccattacttaactcaccctcgggctcccctagcagcctgccctacctctttacctgcttcctggtggagtcagggatgtatacatgagctgctttccctctcagccagaggacatggggggccccagctcccctgcctttccccttctgtgcctggagctgggaagcaggccagggttagctgaggctggctggcaagcagctgggtggtgccagggagagcctgcatagtgccaggtggtgccttgggttccaagctagtccatggccccgataaccttctgcctgtgcacacacctgcccctcactccacccccatcctagctttggtatgggggagagggcacagggccagacaaacctgtgagactttggctccatctctgcaaaagggcgctctgtgagtcagcctgctcccctccaggcttgctcctcccccacccagctctcgtttccaatgcacgtacagcccgtacacaccgtgtgctgggacaccccacagtcagccgcatggctcccctgtgccccagcccctggctccctctgttgatcccggcccctgctccaggcctcactgtgcaactgctgctgtcactgctgcttctggtgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggtaagtggtcatcaatctccaaatccaggttccaggaggttcatgactcccctcccataccccagcctaggctctgttcactcagggaaggaggggagactgtactccccacagaagcccttccagaggtcccataccaatatccccatccccactctcggaggtagaaagggacagatgtggagagaaaataaaaagggtgcaaaaggagagaggtgagctggatgagatgggagagaagggggaggctggagaagagaaagggatgagaactgcagatgagagaaaaaatgtgcagacagaggaaaaaaataggtggagaaggagagtcagagagtttgaggggaagagaaaaggaaagcttgggaggtgaagtgggtaccagagacaagcaagaagagctggtagaagtcatctcatcttaggctacaatgaggaattgagacctaggaagaagggacacagcaggtagagaaacgtggcttcttgactcccaagccaggaatttggggaaaggggttggagaccatacaaggcagagggatgagtggggagaagaaagaagggagaaaggaaagatggtgtactcactcatttgggactcaggactgaagtgcccactcactttttttttttttttttttgagacaaactttcacttttgttgcccaggctggagtgcaatggcgcgatctcggctcactgcaacctccacctcccgggttcaagtgattctcctgcctcagcctctagccaagtagctgcgattacaggcatgcgccaccacgcccggctaatttttgtatttttagtagagacggggtttcgccatgttggtcaggctggtctcgaactcctgatctcaggtgatccaaccaccctggcctcccaaagtgctgggattataggcgtgagccacagcgcctggcctgaagcagccactcacttttacagaccctaagacaatgattgcaagctggtaggattgctgtttggcccacccagctgcggtgttgagtttgggtgcggtctcctgtgctttgcacctggcccgcttaaggcatttgttacccgtaatgctcctgtaaggcatctgcgtttgtgacatcgttttggtcgccaggaagggattggggctctaagcttgagcggttcatccttttcatttatacaggggatgaccagagtcattggcgctatggaggtgagacacccacccgctgcacagacccaatctgggaacccagctctgtggatctcccctacagccgtccctgaacactggtcccgggcgtcccacccgccgcccaccgtcccaccccctcaccttttctacccgggttccctaagttcctgacctaggcgtcagacttcctcactatactctcccaccccaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtggtgagggggtctccccgccgagacttggggatggggcggggcgcagggaagggaaccgtcgcgcagtgcctgcccgggggttgggctggccctaccgggcggggccggctcacttgcctctccctacgcagtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgaggtgagcgcggactggccgagaaggggcaaaggagcggggcggacgggggccagagacgtggccctctcctaccctcgtgtccttttcagatccacgtggttcacctcagcaccgcctttgccagagttgacgaggccttggggcgcccgggaggcctggccgtgttggccgcctttctggaggtaccagatcctggacaccccctactccccgctttcccatcccatgctcctcccggactctatcgtggagccagagaccccatcccagcaagctcactcaggcccctggctgacaaactcattcacgcactgtttgttcatttaacacccactgtgaaccaggcaccagcccccaacaaggattctgaagctgtaggtccttgcctctaaggagcccacagccagtgggggaggctgacatgacagacacataggaaggacatagtaaagatggtggtcacagaggaggtgacacttaaagccttcactggtagaaaagaaaaggaggtgttcattgcagaggaaacagaatgtgcaaagactcagaatatggcctatttagggaatggctacatacaccatgattagaggaggcccagtaaagggaagggatggtgagatgcctgctaggttcactcactcacttttatttatttatttatttttttgacagtctctctgtcgcccaggctggagtgcagtggtgtgatcttgggtcactgcaacttccgcctcccgggttcaagggattctcctgcctcagcttcctgagtagctggggttacaggtgtgtgccaccatgcccagctaatttttttttgtatttttagtagacagggtttcaccatgttggtcaggctggtctcaaactcctggcctcaagtgatccgcctgactcagcctaccaaagtgctgattacaagtgtgagccaccgtgcccagccacactcactgattctttaatgccagccacacagcacaaagttcagagaaatgcctccatcatagcatgtcaatatgttcatactcttaggttcatgatgttcttaacattaggttcataagcaaaataagaaaaaagaataataaataaaagaagtggcatgtcaggacctcacctgaaaagccaaacacagaatcatgaaggtgaatgcagaggtgacaccaacacaaaggtgtatatatggtttcctgtggggagtatgtacggaggcagcagtgagtgagactgcaaacgtcagaagggcacgggtcactgagagcctagtatcctagtaaagtgggctctctccctctctctccagcttgtcattgaaaaccagtccaccaagcttgttggttcgcacagcaagagtacatagagtttgaaataatacataggattttaagagggagacactgtctctaaaaaaaaaaacaacagcaacaacaaaaagcaacaaccattacaattttatgttccctcagcattctcagagctgaggaatgggagaggactatgggaacccccttcatgttccggccttcagccatggccctggatacatgcactcatctgtcttacaatgtcattcccccaggagggcccggaagaaaacagtgcctatgagcagttgctgtctcgcttggaagaaatcgctgaggaaggtcagtttgttggtctggccactaatctctgtggcctagttcataaagaatcaccctttggagcttcaggtctgaggctggagatgggctccctccagtgcaggagggattgaagcatgagccagcgctcatcttgataataaccatgaagctgacagacacagttacccgcaaacggctgcctacagattgaaaaccaagcaaaaaccgccgggcacggtggctcacgcctgtaatcccagcactttgggaggccaaggcaggtggatcacgaggtcaagagatcaagaccatcctggccaacatggtgaaaccccatctctactaaaaatacgaaaaaatagccaggcgtggtggcgggtgcctgtaatcccagctactcgggaggctgaggcaggagaatggcatgaacccgggaggcagaagttgcagtgagccgagatcgtgccactgcactccagcctgggcaacagagcgagactcttgtctcaaaaaaaaaaaaaaaaaagaaaaccaagcaaaaaccaaaatgagacaaaaaaaacaagaccaaaaaatggtgtttggaaattgtcaaggtcaagtctggagagctaaactttttctgagaactgtttatctttaataagcatcaaatattttaactttgtaaatacttttgttggaaatcgttctcttcttagtcactcttgggtcattttaaatctcacttactctactagaccttttaggtttctgctagactaggtagaactctgcctttgcatttcttgtgtctgttttgtatagttatcaatattcatatttatttacaagttattcagatcattttttcttttcttttttttttttttttttttttttacatctttagtagagacagggtttcaccatattggccaggctgctctcaaactcctgaccttgtgatccaccagcctcggcctcccaaagtgctgggattcattttttctttttaatttgctctgggcttaaacttgtggcccagcactttatgatggtacacagagttaagagtgtagactcagacggtctttcttctttccttctcttccttcctcccttccctcccaccttcccttctctccttcctttctttcttcctctcttgcttcctcaggcctcttccagttgctccaaagccctgtacttttttttgagttaacgtcttatgggaagggcctgcacttagtgaagaagtggtctcagagttgagttaccttggcttctgggaggtgaaactgtatccctataccctgaagctttaagggggtgcaatgtagatgagaccccaacatagatcctcttcacaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcaggtgggcctggggtgtgtgtggacacagtgggtgcgggggaaagaggatgtaagatgagatgagaaacaggagaagaaagaaatcaaggctgggctctgtggcttacgcctataatcccaccacgttgggaggctgaggtgggagaatggtttgagcccaggagttcaagacaaggcggggcaacatagtgtgaccccatctctaccaaaaaaaccccaacaaaaccaaaaatagccgggcatggtggtatgcggcctagtcccagctactcaaggaggctgaggtgggaagatcgcttgattccaggagtttgagactgcagtgagctatgatcccaccactgcctaccatctttaggatacatttatttatttataaaagaaatcaagaggctggatggggaatacaggagctggagggtggagccctgaggtgctggttgtgagctggcctgggacccttgtttcctgtcatgccatgaacccacccacactgtccactgacctccctagctccacaccctctctgacaccctgtggggacctggtgactctcggctacagctgaacttccgagcgacgcagcctttgaatgggcgagtgattgaggcctccttccctgctggagtggacagcagtcctcgggctgctgagccaggtacagctttgtctggtttccccccagccagtagtcccttatcctcccatgtgtgtgccagtgtctgtcattggtggtcacagcccgcctctcacatctcctttttctctccagtccagctgaattcctgcctggctgctggtgagtctgcccctcctcttggtcctgatgccaggagactcctcagcaccattcagccccagggctgctcaggaccgcctctgctccctctccttttctgcagaacagaccccaaccccaatattagagaggcagatcatggtggggattcccccattgtccccagaggctaattgattagaatgaagcttgagaaatctcccagcatccctctcgcaaaagaatccccccccctttttttaaagatagggtctcactctgtttgccccaggctggggtgttgtggcacgatcatagctcactgcagcctcgaactcctaggctcaggcaatcctttcaccttagcttctcaaagcactgggactgtaggcatgagccactgtgcctggccccaaacggcccttttacttggcttttaggaagcaaaaacggtgcttatcttaccccttctcgtgtatccaccctcatcccttggctggcctcttctggagactgaggcactatggggctgcctgagaactcggggcaggggtggtggagtgcactgaggcaggtgttgaggaactctgcagacccctcttccttcccaaagcagccctctctgctctccatcgcaggtgacatcctagccctggtttttggcctcctttttgctgtcaccagcgtcgcgttccttgtgcagatgagaaggcagcacaggtattacactgaccctttcttcaggcacaagcttcccccacccttgtggagtcacttcatgcaaagcgcatgcaaatgagctgctcctgggccagttttctgattagcctttcctgttgtgtacacacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataataaaatatgtgttagtcacctttgttccccaaatcagaaggaggtatttgaatttcctattactgttattagcaccaatttagtggtaatgcatttattctattacagttcggcctccttccacacatcactccaatgtgttgctcc

SEQ ID NO: 4 - protein, 37 aa, Homo sapiens
MAPLCPSPWLPLLIPAPAPGLTVQLLLSLLLLMPVHP

SEQ ID NO: 5 - protein, 377 aa, Homo sapiens
QRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGD

SEQ ID NO: 6 - protein, 20 aa, Homo sapiens
ILALVFGLLFAVTSVAFLVQ

SEQ ID NO: 7 - protein, 25 aa, Homo sapiens
MRRQHRRGTKGGVSYRPAEVAETGA

SEQ ID NO: 8 - protein, 59 aa, Homo sapiens
SSGEDDPLGEEDLPSEEDSPREEDPPGEEDLPGEEDLPGEEDLPEVKPKSEEEGSLKLE

SEQ ID NO: 9 - protein, 257 aa, Homo sapiens
GDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFP

SEQ ID NO: 10 - protein, 59 aa, Homo sapiens
SASEEPSPSEVPFPSEEPSPSEEPFPSVRPFPSVVLFPSEEPFPSKEPSPSEEPSASEE

SEQ ID NO: 11 - protein, 7 aa, Homo sapiens
PGEEDLP

SEQ ID NO: 12 - protein, 36 aa, Homo sapiens
GEEDLPSEEDSPREEDPPGEEDLPGEEDLPGEEDLP

SEQ ID NO: 13 - protein, 6 aa, Homo sapiens
GEEDLP

SEQ ID NO: 14 - protein, 4 aa, Homo sapiens
EEDL

SEQ ID NO: 15 - protein, 5 aa, Homo sapiens
EEDLP

SEQ ID NO: 16 - protein, 6 aa, Homo sapiens
EDLPSE

SEQ ID NO: 17 - protein, 7 aa, Homo sapiens
EEDLPSE

SEQ ID NO: 18 - protein, 6 aa, Homo sapiens
DLPGEE

SEQ ID NO: 19 - protein, 6 aa, Homo sapiens
EEDLPS

SEQ ID NO: 20 - protein, 6 aa, Homo sapiens
GEDDPL

SEQ ID NO: 21 - protein, 16 aa, Homo sapiens
HPQRLPRMQEDSPLGG

SEQ ID NO: 22 - protein, 24 aa, Homo sapiens
EEDSPREEDPPGEEDLPGEEDLPG

SEQ ID NO: 23 - protein, 21 aa, Homo sapiens
NNAHRDKEGDDQSHWRYGGDP

SEQ ID NO: 24 - DNA, 540 bp, Homo sapiens
  promoter (1)..(540)
cttgcttttcattcaagctcaagtttgtctcccacatacccattacttaactcaccctcgggctcccctagcagcctgccctacctctttacctgcttcctggtggagtcagggatgtatacatgagctgctttccctctcagccagaggacatggggggccccagctcccctgcctttccccttctgtgcctggagctgggaagcaggccagggttagctgaggctggctggcaagcagctgggtggtgccagggagagcctgcatagtgccaggtggtgccttgggttccaagctagtccatggccccgataaccttctgcctgtgcacacacctgcccctcactccacccccatcctagctttggtatgggggagagggcacagggccagacaaacctgtgagactttggctccatctctgcaaaagggcgctctgtgagtcagcctgctcccctccaggcttgctcctcccccacccagctctcgtttccaatgcacgtacagcccgtacacaccgtgtgctgggacaccccacag

SEQ ID NO: 25 - DNA, 445 bp, Homo sapiens
gcccgtacacaccgtgtgctgggacaccccacagtcagccgcatggctcccctgtgccccagcccctggctccctctgttgatcccggcccctgctccaggcctcactgtgcaactgctgctgtcactgctgcttctggtgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaag

SEQ ID NO: 26 - DNA, 30 bp, Homo sapiens
gggatgaccagagtcattggcgctatggag

SEQ ID NO: 27 - DNA, 171 bp, Homo sapiens
gcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtg

SEQ ID NO: 28 - DNA, 143 bp, Homo sapiens
tgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgag

SEQ ID NO: 29 - DNA, 93 bp, Homo sapiens
atccacgtggttcacctcagcaccgcctttgccagagttgacgaggccttggggcgcccgggaggcctggccgtgttggccgcctttctggag

SEQ ID NO: 30 - DNA, 67 bp, Homo sapiens
gagggcccggaagaaaacagtgcctatgagcagttgctgtctcgcttggaagaaatcgctgaggaag

SEQ ID NO: 31 - DNA, 158 bp, Homo sapiens
gctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcag

SEQ ID NO: 32 - DNA, 145 bp, Homo sapiens
ctccacaccctctctgacaccctgtggggacctggtgactctcggctacagctgaacttccgagcgacgcagcctttgaatgggcgagtgattgaggcctccttccctgctggagtggacagcagtcctcgggctgctgagccag

SEQ ID NO: 33 - DNA, 27 bp, Homo sapiens
tccagctgaattcctgcctggctgctg

SEQ ID NO: 34 - DNA, 82 bp, Homo sapiens
gtgacatcctagccctggtttttggcctcctttttgctgtcaccagcgtcgcgttccttgtgcagatgagaaggcagcacag

SEQ ID NO: 35 - DNA, 191 bp, Homo sapiens
aaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataat

SEQ ID NO: 36 - DNA, 1174 bp, Homo sapiens
gtaagtggtcatcaatctccaaatccaggttccaggaggttcatgactcccctcccataccccagcctaggctctgttcactcagggaaggaggggagactgtactccccacagaagcccttccagaggtcccataccaatatccccatccccactctcggaggtagaaagggacagatgtggagagaaaataaaaagggtgcaaaaggagagaggtgagctggatgagatgggagagaagggggaggctggagaagagaaagggatgagaactgcagatgagagaaaaaatgtgcagacagaggaaaaaaataggtggagaaggagagtcagagagtttgaggggaagagaaaaggaaagcttgggaggtgaagtgggtaccagagacaagcaagaagagctggtagaagtcatctcatcttaggctacaatgaggaattgagacctaggaagaagggacacagcaggtagagaaacgtggcttcttgactcccaagccaggaatttggggaaaggggttggagaccatacaaggcagagggatgagtggggagaagaaagaagggagaaaggaaagatggtgtactcactcatttgggactcaggactgaagtgcccactcactttttttttttttttttttgagacaaactttcacttttgttgcccaggctggagtgcaatggcgcgatctcggctcactgcaacctccacctcccgggttcaagtgattctcctgcctcagcctctagccaagtagctgcgattacaggcatgcgccaccacgcccggctaatttttgtatttttagtagagacggggtttcgccatgttggtcaggctggtctcgaactcctgatctcaggtgatccaaccaccctggcctcccaaagtgctgggattataggcgtgagccacagcgcctggcctgaagcagccactcacttttacagaccctaagacaatgattgcaagctggtaggattgctgtttggcccacccagctgcggtgttgagtttgggtgcggtctcctgtgctttgcacctggcccgcttaaggcatttgttacccgtaatgctcctgtaaggcatctgcgtttgtgacatcgttttggtcgccaggaagggattggggctctaagcttgagcggttcatccttttcatttatacag

SEQ ID NO: 37 - DNA, 193 bp, Homo sapiens
gtgagacacccacccgctgcacagacccaatctgggaacccagctctgtggatctcccctacagccgtccctgaacactggtcccgggcgtcccacccgccgcccaccgtcccaccccctcaccttttctacccgggttccctaagttcctgacctaggcgtcagacttcctcactatactctcccaccccag

SEQ ID NO: 38 - DNA, 131 bp, Homo sapiens
gtgagggggtctccccgccgagacttggggatggggcggggcgcagggaagggaaccgtcgcgcagtgcctgcccgggggttgggctggccctaccgggcggggccggctcacttgcctctccctacgcag

SEQ ID NO: 39 - DNA, 89 bp, Homo sapiens
gtgagcgcggactggccgagaaggggcaaaggagcggggcggacgggggccagagacgtggccctctcctaccctcgtgtccttttcag

SEQ ID NO: 40 - DNA, 1400 bp, Homo sapiens
gtaccagatcctggacaccccctactccccgctttcccatcccatgctcctcccggactctatcgtggagccagagaccccatcccagcaagctcactcaggcccctggctgacaaactcattcacgcactgtttgttcatttaacacccactgtgaaccaggcaccagcccccaacaaggattctgaagctgtaggtccttgcctctaaggagcccacagccagtgggggaggctgacatgacagacacataggaaggacatagtaaagatggtggtcacagaggaggtgacacttaaagccttcactggtagaaaagaaaaggaggtgttcattgcagaggaaacagaatgtgcaaagactcagaatatggcctatttagggaatggctacatacaccatgattagaggaggcccagtaaagggaagggatggtgagatgcctgctaggttcactcactcacttttatttatttatttatttttttgacagtctctctgtcgcccaggctggagtgcagtggtgtgatcttgggtcactgcaacttccgcctcccgggttcaagggattctcctgcctcagcttcctgagtagctggggttacaggtgtgtgccaccatgcccagctaatttttttttgtatttttagtagacagggtttcaccatgttggtcaggctggtctcaaactcctggcctcaagtgatccgcctgactcagcctaccaaagtgctgattacaagtgtgagccaccgtgcccagccacactcactgattctttaatgccagccacacagcacaaagttcagagaaatgcctccatcatagcatgtcaatatgttcatactcttaggttcatgatgttcttaacattaggttcataagcaaaataagaaaaaagaataataaataaaagaagtggcatgtcaggacctcacctgaaaagccaaacacagaatcatgaaggtgaatgcagaggtgacaccaacacaaaggtgtatatatggtttcctgtggggagtatgtacggaggcagcagtgagtgagactgcaaacgtcagaagggcacgggtcactgagagcctagtatcctagtaaagtgggctctctccctctctctccagcttgtcattgaaaaccagtccaccaagcttgttggttcgcacagcaagagtacatagagtttgaaataatacataggattttaagagggagacactgtctctaaaaaaaaaaacaacagcaacaacaaaaagcaacaaccattacaattttatgttccctcagcattctcagagctgaggaatgggagaggactatgggaacccccttcatgttccggccttcagccatggccctggatacatgcactcatctgtcttacaatgtcattcccccag

SEQ ID NO: 41 - DNA, 1334 bp, Homo sapiens
gtcagtttgttggtctggccactaatctctgtggcctagttcataaagaatcaccctttggagcttcaggtctgaggctggagatgggctccctccagtgcaggagggattgaagcatgagccagcgctcatcttgataataaccatgaagctgacagacacagttacccgcaaacggctgcctacagattgaaaaccaagcaaaaaccgccgggcacggtggctcacgcctgtaatcccagcactttgggaggccaaggcaggtggatcacgaggtcaagagatcaagaccatcctggccaacatggtgaaaccccatctctactaaaaatacgaaaaaatagccaggcgtggtggcgggtgcctgtaatcccagctactcgggaggctgaggcaggagaatggcatgaacccgggaggcagaagttgcagtgagccgagatcgtgccactgcactccagcctgggcaacagagcgagactcttgtctcaaaaaaaaaaaaaaaaaagaaaaccaagcaaaaaccaaaatgagacaaaaaaaacaagaccaaaaaatggtgtttggaaattgtcaaggtcaagtctggagagctaaactttttctgagaactgtttatctttaataagcatcaaatattttaactttgtaaatacttttgttggaaatcgttctcttcttagtcactcttgggtcattttaaatctcacttactctactagaccttttaggtttctgctagactaggtagaactctgcctttgcatttcttgtgtctgttttgtatagttatcaatattcatatttatttacaagttattcagatcattttttcttttcttttttttttttttttttttttttacatctttagtagagacagggtttcaccatattggccaggctgctctcaaactcctgaccttgtgatccaccagcctcggcctcccaaagtgctgggattcattttttctttttaatttgctctgggcttaaacttgtggcccagcactttatgatggtacacagagttaagagtgtagactcagacggtctttcttctttccttctcttccttcctcccttccctcccaccttcccttctctccttcctttctttcttcctctcttgcttcctcaggcctcttccagttgctccaaagccctgtacttttttttgagttaacgtcttatgggaagggcctgcacttagtgaagaagtggtctcagagttgagttaccttggcttctgggaggtgaaactgtatccctataccctgaagctttaagggggtgcaatgtagatgagaccccaacatagatcctcttcacag

SEQ ID NO: 42 - DNA, 512 bp, Homo sapiens
gtgggcctggggtgtgtgtggacacagtgggtgcgggggaaagaggatgtaagatgagatgagaaacaggagaagaaagaaatcaaggctgggctctgtggcttacgcctataatcccaccacgttgggaggctgaggtgggagaatggtttgagcccaggagttcaagacaaggcggggcaacatagtgtgaccccatctctaccaaaaaaaccccaacaaaaccaaaaatagccgggcatggtggtatgcggcctagtcccagctactcaaggaggctgaggtgggaagatcgcttgattccaggagtttgagactgcagtgagctatgatcccaccactgcctaccatctttaggatacatttatttatttataaaagaaatcaagaggctggatggggaatacaggagctggagggtggagccctgaggtgctggttgtgagctggcctgggacccttgtttcctgtcatgccatgaacccacccacactgtccactgacctccctag

SEQ ID NO: 43 - DNA, 114 bp, Homo sapiens
gtacagctttgtctggtttccccccagccagtagtcccttatcctcccatgtgtgtgccagtgtctgtcattggtggtcacagcccgcctctcacatctcctttttctctccag

SEQ ID NO: 44 - DNA, 617 bp, Homo sapiens
gtgagtctgcccctcctcttggtcctgatgccaggagactcctcagcaccattcagccccagggctgctcaggaccgcctctgctccctctccttttctgcagaacagaccccaaccccaatattagagaggcagatcatggtggggattcccccattgtccccagaggctaattgattagaatgaagcttgagaaatctcccagcatccctctcgcaaaagaatccccccccctttttttaaagatagggtctcactctgtttgccccaggctggggtgttgtggcacgatcatagctcactgcagcctcgaactcctaggctcaggcaatcctttcaccttagcttctcaaagcactgggactgtaggcatgagccactgtgcctggccccaaacggcccttttacttggcttttaggaagcaaaaacggtgcttatcttaccccttctcgtgtatccaccctcatcccttggctggcctcttctggagactgaggcactatggggctgcctgagaactcggggcaggggtggtggagtgcactgaggcaggtgttgaggaactctgcagacccctcttccttcccaaagcagccctctctgctctccatcgcag

SEQ ID NO: 45 - DNA, 130 bp, Homo sapiens
gtattacactgaccctttcttcaggcacaagcttcccccacccttgtggagtcacttcatgcaaagcgcatgcaaatgagctgctcctgggccagttttctgattagcctttcctgttgtgtacacacag

SEQ ID NO: 46 - DNA, 11 bp, Homo sapiens
agaaggtaagt

SEQ ID NO: 47 - DNA, 11 bp, Homo sapiens
tggaggtgaga

SEQ ID NO: 48 - DNA, 11 bp, Homo sapiens
cagtcgtgagg

SEQ ID NO: 49 - DNA, 11 bp, Homo sapiens
ccgaggtgagc

SEQ ID NO: 50 - DNA, 11 bp, Homo sapiens
tggaggtacca

SEQ ID NO: 51 - DNA, 11 bp, Homo sapiens
ggaaggtcagt

SEQ ID NO: 52 - DNA, 11 bp, Homo sapiens
agcaggtgggc

SEQ ID NO: 53 - DNA, 11 bp, Homo sapiens
gccaggtacag

SEQ ID NO: 54 - DNA, 11 bp, Homo sapiens
tgctggtgagt

SEQ ID NO: 55 - DNA, 11 bp, Homo sapiens
cacaggtatta

SEQ ID NO: 56 - DNA, 11 bp, Homo sapiens
atacaggggat

SEQ ID NO: 57 - DNA, 11 bp, Homo sapiens
ccccaggcgac

SEQ ID NO: 58 - DNA, 11 bp, Homo sapiens
acgcagtgcaa

SEQ ID NO: 59 - DNA, 11 bp, Homo sapiens
tttcagatcca

SEQ ID NO: 60 - DNA, 11 bp, Homo sapiens
ccccaggaggg

SEQ ID NO: 61 - DNA, 11 bp, Homo sapiens
tcacaggctca

SEQ ID NO: 62 - DNA, 11 bp, Homo sapiens
ccctagctcca

SEQ ID NO: 63 - DNA, 11 bp, Homo sapiens
ctccagtccag

SEQ ID NO: 64 - DNA, 12 bp, Homo sapiens
tcgcaggtgaca

SEQ ID NO: 65 - DNA, 11 bp, Homo sapiens
acacagaaggg

SEQ ID NO: 66 - RNA, 470 nt, Homo sapiens
cauggccccgauaaccuucugccugugcacacaccugccccucacuccacccccauccuagcuuugguaugggggagagggcacagggccagacaaaccugugagacuuuggcuccaucucugcaaaagggcgcucugugagucagccugcuccccuccaggcuugcuccucccccacccagcucucguuuccaaugcacguacagcccguacacaccgugugcugggacaccccacagucagccgcauggcuccccugugccccagccccuggcucccucuguugaucccggccccugcuccaggccucacugugcaacugcugcugucacugcugcuucuggugccuguccauccccagagguugccccggaugcaggaggauucccccuugggaggaggcucuucuggggaagaugacccacugggcgaggaggaucugcccagugaagaggauucacccagagagg

SEQ ID NO: 67 - protein, 13 aa, Homo sapiens
LEEGPEENSAYEQ

SEQ ID NO: 68 - protein, 16 aa, Homo sapiens
MRRQHRRGTKGGVSYR

SEQ ID NO: 69 - protein, 232 aa, Homo sapiens
IRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSS

SEQ ID NO: 70 - DNA, 1552 bp, Homo sapiens
  ref <308>: DATABASE ACCESSION NUMBER: EMBL Accession No. X66839
  ref <309>: DATABASE ENTRY DATE: 1995-10-10
  ref <313>: RELEVANT RESIDUES IN SEQ ID NO: (1)..(1552)
gcccgtacacaccgtgtgctgggacaccccacagtcagccgcatggctcccctgtgccccagcccctggctccctctgttgatcccggcccctgctccaggcctcactgtgcaactgctgctgtcactgctgcttctgatgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataat

SEQ ID NO: 71 - DNA, 1965 bp, Mus musculus
acagtgtgtcctgggacaccccagtcagctgcatggcctccctgggccccagcccctgggcccctttgtcgaccccagcccctactgcacagctgctgctgttcctgctgctccaagtgtctgctcagccccagggcctgtccgggatgcagggggagccctccttgggagacagttcatctggggaggatgagctgggcgtggatgttctgcccagtgaagaggatgcacctgaagaggcggatccacccgatggggaggatccacctgaagttaattctgaagacaggatggaggagtcccttgggttagaggatctatcgactcccgaggctcctgagcacagtcaaggttcccacggggatgaaaaagggggtggccacagtcattggagctatggaggcaccctactctggccccaggtgtccccagcctgtgctggccgctttcagtccccggtagacatccgcctggagcgcaccgcattctgccgcactctgcaacccctggaacttctgggttatgagctccagcctctcccggaactgagcctatccaacaatggccacacggtacagctgactctgccgccggggctgaagatggctctgggacctgggcaggaataccgggccttgcagttgcatctacactggggaacttcagatcacccaggctcagaacacacagtcaatggtcaccgtttccctgctgagatccacgtggtgcacctcagtactgctttctccgaacttcatgaagccttgggccgcccaggaggcctggcagttttggctgcctttctgcaggagagcccagaagaaaacagtgcttacgaacagttgctgtcccatttggaagaaatctcggaggaaggctccaagattgagatcccaggtttggatgtatctgcactgttgccctcggacctcagccgctactaccgatatgaagggtctctgaccacacctccctgcagccagggggtcatctggactgtgttcaatgagacagtgaaactgagcgctaagcagctccatactctctccgtttccttgtggggacctcgtgattctcggctacaactgaacttccgagccacgcaacccttgaatgggcgaacgattgaggcttccttccctgctgcagaggatagcagccctgagccagtccatgtgaattcctgcttcactgctggtgacatcctagccttggtgtttggccttctctttgctgtcaccagcatcgccttcctcctacagctgaggaggcagcacaggcacagaagtgggaccaaagatagagttagctacagtccggcagagatgacagaaactggagcctaataaggagaaaccagctaaaggaatctggaaaccttgtcctgtcctgcctgttatacagcctcctttttaaccactacgaaatcatttccatctttttaactttcttctttccttaaagtactgtgataggagctaaccgtgaaaaaacaagattgggctcaattcttgctgggttttgatggttttgaagacagggtttctctgtgtagccctggctgtcctgggactcactctgtagcctaggctggccttgaactcagagatccgattgcctctgccttcttagtgccaggactgagggtatgtaccactaccaccgccaccccgcaattcttgcttttattctgcagtcaaatgctccaccactgagctacacacccctccatcttactatgcaggcagactggccttgcattgtgtatgtatggtgatggagtttgaagccagggtcttacctactaggcaagcgttctatcactgacctaagcctctctagtcccttctcttgaaccctctattctcctgtcccagcctcttgaatgctgggattacaggaatgcaccatcatgcttggtcatcccaattatttaaaaaaaatatataaatcttcctagtaaaa

SEQ ID NO: 72 - DNA, 8725 bp, Mus musculus
  misc_feature (1434)..(1434): n is a, c, g, or t
  misc_feature (1470)..(1470): n is a, c, g, or t
  misc_feature (1482)..(1482): n is a, c, g, or t
  misc_feature (1634)..(1634): n is a, c, g, or t
  misc_feature (1655)..(1655): n is a, c, g, or t
  misc_feature (1734)..(1734): n is a, c, g, or t
  misc_feature (1752)..(1752): n is a, c, g, or t
  misc_feature (3057)..(3057): n is a, c, g, or t
  misc_feature (3499)..(3499): n is a, c, g, or t
  misc_feature (3781)..(3781): n is a, c, g, or t
  misc_feature (4535)..(4535): n is a, c, g, or t
  misc_feature (6708)..(6708): n is a, c, g, or t
  misc_feature (6923)..(6923): n is a, c, g, or t
  misc_feature (7020)..(7020): n is a, c, g, or t
  misc_feature (7087)..(7087): n is a, c, g, or t
  ref <308>: DATABASE ACCESSION NUMBER: EMBL Accession No. AY049077
  ref <309>: DATABASE ENTRY DATE: 2002-12-19
  ref <313>: RELEVANT RESIDUES IN SEQ ID NO: (1)..(8725)
aaatataatgcaggcaaaacagcaatgcacatgaaataaaaataaccaagtttaaacctggttaggtccctcaaataaagttccttggacccttctgtatagatactaccttttatttagatcaggaatcatctagaaggctctcaaagagaagatgggatagcagggttacctagagccatgggatgtagctgtatgtgtccaggaataaaaataactcaatgcattcaattaacctagacacccacactggtggatgctcgttgcatcattttagatacctggatgaaacgaccttattcttttatagttgctgaggctatgggaatctcctgggaatgggagctgcagagcttaaggctgtctgctattctgtgctgcgtgcctggagacaccagcagagtcaaggacactgggagaggcttggtacatggtcactagatggacagattgggagctccatccgttctcactctgtcctgattcataaagaatcaggctgcctgtgaaatccacactcatagttgtcttccacatggcctgatgtaagaactggtctctgtcacgctcttcaaaagactttattttacatctatttggtgtatgtgtgtgtgtgtgcacatgcatgtgccatggcacacacagggggcagagggcaacttgtagtacaggttctctctttccaccatgtcgattttggggttccacctcagatcatcagattcggtggcaagtgtctttacccactaagcctctgaccagtctctattcaaaatggtccatgtctcttctttgtccagaattccaatctctttactgtgattattaaccaacccctccccgtggggcttctacctctgcttcaaagatgggaacagattttacccacatccatagttccttgaggcagaattatgacatctgtcctcactcgctggagcgagagcttctcagataggagccttcgttttcatttccgcatccctagtccacagcccagtgcctaacataccgtaggtattaaacaaatacggcgtgcaaacatgaatggcattctgggcaacaagacggagattcacaaaggcagtgcagagtttggagagacgaatgaatagtttagctctgggagcaaagctcaagtgaaagaatgcttgcatggcacccatggccccctggaatcagtctccagcactgaaaaagaaacaaacaaacaaagcaaaaaatgcatgcgcgcaggagcacacacacacaaattatctgaccagaacattaggtggaagagcaagagacaagactggtttcttgggtgaggttttgaagacctggaagtcgggaaataaaatgatcagactttggcaggcagtgggggggggggggtaaccattagagtctcatatgtagattcaaataatcatgctcaaatttgccttttanttagctgtgagcccctccacctattcacccctaggntcctccagggtnttaacctgcccccttacccaagacctgactgggccaggatacatgcatggatttcttgcctcctcagcccaactccacgggggcccagcttctttctttcccctgccctgccagggccgggaagcaggccaggcttagcagaggccaggagntgggtggtgccagggagaacntatacagtgccaggtggtgccttgggttccaagctgagctggtgatcctgataaccttctttctgcctatgaacacacntgcccctcaccccaaccntacccattaccgcaccaatgtccaggctttgatatcggggagggggcatagggccagacagaacctgtgggactttggctccatctctacaaaagggcactgtgagtcagcctgctcccctcaaggcttgctcctcccctacccagctcctatttccgatgcacgtacagcccgtccacagtgtgtcctgggacaccccagtcagctgcatggcctccctgggccccagcccctgggcccctttgtcgaccccagcccctactgcacagctgctgctgttcctgctgctccaagtgtctgctcagccccagggcctgtccgggatgcagggggagccctccttgggagacagttcatctggggaggatgagctgggcgtggatgttctgcccagtgaagaggatgcacctgaagaggcggatccacccgatggggaggatccacctgaagttaattctgaagacaggatggaggagtcccttgggttagaggatctatcgactcccgaggctcctgagcacagtcaaggttcccacggggatgaaaaaggtaagtggtcattaacttccaaatctaggctcaggtaagtttatgccttgtcctcccccactcccctctcttccccgaaatgctagctgagagcattcggcaattatactctaataggtagtctttcccccctaaaaggagagaagaaaaaaaaagccgagaatagggggaaattaaagaggaaatagaactgggaatggacagagggggaaaataagtgtgtgtgtgtgtgtgtgtgtgtgtgtgtgtgaagagagggaggtagagagaatgagagacagacagacagagacagacacagagagacagagacagagagatgaaaagggaagagaagggggctcctggaagacacacaaggagctcacagccgttactcacctatgatttgcggtgtgggaaaagacaggtggaggaactttggtttgctctgggggcggaggatgagataaggtcttattgtgtagatcggattggccttgaactagttctgccctttaccttcttcttcctccgagttctgggatttcaggtgtgtgcgccaccgtgcttggctctaagccaggaactttagacaggaaagagagatgagtgggaaggtgggtgagtaagggaaaaagaacgatgatttggatctcaaactgaagccgtgcctacagaccctagtttgcaaactatcggtagggctactgatggctcctgcagtgatggttgcggtgggcggaattcccagctggtggttaatccacttgtgcntcacaccaaccctggctacattccctttctattacgtgcaacactcttgtaagacatccggggctgtgaggccccctgccagagcgggagggaatatttttttattctgggcactgggaagggactgggctgtaagagtgggcttattaattcttctcatttatgcagggggtggccacagtcattggagctatggaggtaagacacggccggctacacagacccaatctgggagcccagccctggtgatctgccgcaccaacattcccagtagtagccctagactggacccaggtcctactctatggcccccctcccgcgccccgcactcccccacgccagtccctggagcgcccccccttcctcccaaccccaagcgtcctgattccaacccttcatttatcctaacggttcttgtccaaatcgctagatttcttgcacnttttcttttcatcccaggcaccctactctggccccaggtgtccccagcctgtgctggccgctttcagtccccggtagacatccgcctggagcgcaccgcattctgccgcactctgcaacccctggaacttctgggttatgagctccagcctctcccggaactgagcctatccaacaatggccacacgggtgaggtgtctggggcgggacctatggaggggagtggcaatcaacgtgtgcacttaggagttctgctggtgctcacgctcacgtgtgggtaganttctcacttgccactccccacgcagtacagctgactctgccgccggggctgaagatggctctgggacctgggcaggaataccgggccttgcagttgcatctacactggggaacttcagatcacccaggctcagaacacacagtcaatggtcaccgtttccctgctgaggtggggactgggctgtcccaagaagaagcaagagggtatgatcagaggtctggccactatctgatccttccatcctctctagatccacgtggtgcacctcagtactgctttctccgaacttcatgaagccttgggccgcccaggaggcctggcagttttggctgcctttctgcaggtaccctatttgacattcacgtcctgtgggttcatgcatctggctagcagagacaccattccaagcacaagcgcaagctggtcaattagctctcagccccattcactcctttgttcattcactcagcaccagcagggaaccaggagcagactaacacaaggcttcctttaggaggaaggctgtcttgacagtcataagtgacgaacacggaaaaaataatggccacagaggaggcgaaacttaaatctttcactgatgggaaaccaaggaggaatactgcacagagagaggtcagcgtgagcaaggctggagtatggaccagttagggagtgccacagcaacaagcattgccctgggaagagccatttctctgctgcttctgtggcccctcacttccccttcatgaagtanaagtattcaagtgacctctttgtacctgtaataatcaagaacgtacactccaaggtgggaggaagccaggaagtgaggacgcctatgagacttaccagtgtgacattcacagcacaaagttcagggaagttcctcaggagcaggcgggcaggcgtagttattaaagtgaacgcaaaggcagtacaagtataacatacatgatggtttttttcaggaaaccacggcgtgtgtgtgtgtgtgtgtgtgtgtgtgtgtgcgagagagagagagagagagagagagagagagagagagagagagagagagagagtgtaaatagtgatgagtgaaacgatggacctagatcttgagggccaggcagcctgggaaactttctccccttcagtttgccattgctaattcatttgcaaagcaagagtaatagaacttgaataagattgtatattccccagcatccttagaggtgaggaacggggaaagactatgggaaccccatcccatgcccaggcttccagctgtagtcctagaaatgggtatcaattggccttgtactatatcccgtgttcacccctgcaggagagcccagaagaaaacagtgcttacgaacagttgctgtcccatttggaagaaatctcggaggaaggttggtggcttgcccagtgaacaatgaacatagatggagggagctgagggtgaactggctctgactggtgatagctaaactgatgagaaacacaggggatagggaaactactgtccacaggcttgacaattagaatcaaagccaaacagaaaataacattagagaaaacaagcaaagctgagtctggccgtggtggcctccctggtctatagagtgagttccaggccggtcagggttacacagtaagacctggcctcaaacacaaacaaaacaaaaagcaaaatagagaaaacaagccaaagaaaatgtttaaaatttataagactgagtatggaaagctaaactttttttgtttgagaactgtctatcatttatacatatcaaatatttatatattttttcttattttggtttttcgagacagggtttttctgtgtagccctggctggcctggaacttactctgtagtccaggctggcctcgaactcagaaatccgcctgcctctgcctcccaagtgctgggattaaaggcgtggccaccacgcccagctgatgaatgattttgagcaggtcatggaagagcatgctgataataccaacgtttgggaggttggctactcaccaagccagcctaggttacctgaaatcataaagactagggctagggctgaagagagaaggctcaggggttaagagcactgactgctctcccagaggtccaaagtttaattcccaaccacatggtggctcacaaccatctgtaatgagacctgatgccctcttctggtgtgtccgaagacagctacagtgtactcatatacattaaatagatagtatttggaagaaaaaaaaaaaaaggctagggctatagctctgagtagagcaagaacatagcatgcatgaagccttaggtccagagctgcaaaaacactttagtcaaaaagcttgttcccttccatgactgagccattttaaacctcactctactagactttttaggtgttttgataaactggaactcatagaacttttgtgtctgtttcttatcgttgcaaatattacatgtatttacaagttattctacttgtctatctgtgtatccacgggaatgtgctcagtagatagaataaccccccacccccccacccccagtacccctcctccccgagctacattccaattccgctttattccagtctggggttaaccgcggtcccccccttccccaccccccgcacccctcctccccgagctacattccaattccgctttattccagtctggggttaagttagcagttgtgtattcagatgattgaaggtaccacaggagggcttatgagcttttggcttgaggctggaaagatggctcagcagtacaaggcctcctctaactgcccgaagccctttacttccttttggattatccacacatagtaagaagcattgaaactatgcttgtcccgaaagcttccaaggggcagcagtgggggggggggggggggaacgggcccgaggaggcagggtgatntaggtgagacttcacacaatcccaatccgtaggctccaagattgagatcccaggtttggatgtatctgcactgttgccctcggacctcagccgctactaccgatatgaagggtctctgaccacacctccctgcagccagggggtcatctggactgtgttcaatgagacagtgaaactgagcgctaagcaggttagaatggggcgtggtattgacntggggtgttggaaaatgggaatgtgggaaaaaaggaggaaggggtcatgcagtaaagatagacagagggttggggtccccttgtttctttttgtctngagcctgtactatccatcgacccccacttccccagctccatactctctccgtttccttgtggggacntcgtgattctcggctacaactgaacttccgagccacgcaacccttgaatgggcgaacgattgaggcttccttccctgctgcagaggatagcagccctgagccaggtgcccagttatctgatagcccacgttgacatatccccagtcaggggtcctgcctcttcctgtgtcttcatttgtaccagcctgtcccaggtactcatgggccctggttctaatgtctccttttatctcttcagtccatgtgaattcctgcttcactgctggtgagtctgggtcccttgttcctggtgccagtggcgggagggggccacacccttttagtatttcagcccaggagctagtctttactttttctgtagaataaacttgatccccaaatcgatgtgatcggtcaagtaagagacctgtacctctggcttagttgtctgaatgaggctccagaaatctcctgtgagaccccctcccccaaggcacctttcacctagctgctggaagggagaagagtgctttacccttctcactatctgcctccattctccagatggcctgttctgccgaccgaggccctgggggctgcctgaacactcactggggaaagggtcaggcaggagtatgggaagccagcttgcagggaatcccctggcatccccagaagcagctgcctctccccttccaggtgacatcctagccttggtgtttggccttctctttgctgtcaccagcatcgccttcctcctacagctgaggaggcagcacaggtatctatgaccctccttggcatagcattcttgtataacccagaattcccggactgcaaatgagctctcatgcggagtcggctttctgattctcgttccttgttctataggcacagaagtgggaccaaagatagagttagctacagtccggcagagatgacagaaactggagcctaataaggagaaaccagctaaaggaatctggaaaccttgtcctgtcctgcctgttatacagcctcctttttaaccactacgaaatcatttccatctttttaactttcttctttccttaaagtactgtgataggagctaaccgtgaaaaaacaagattgggctcaattcttgctgggttttgatggttttgaagacagggtttctctgtgtagccctggctgtcctgggactcactctgtagcctaggctggccttgaactcagagatccgattgcctctgccttcttagtgccaggactgagggtatgtaccactaccaccgccaccccgcaattcttgcttttattctgcagtcaaatgctccaccactgagctacacacccctccatcttactatgcaggcagactggccttgcattgtgtatgtatggtgatggagtttgaagccagggtcttacctactaggcaagcgttctatcactgacctaagcctctctagtcccttctcttgaaccctctattctcctgtcccagcctcttgaatgctgggattacaggaatgcaccatcatgcttggtcatcccaattatttaaaaaaaatatataaatcttcctagtaaaactcgtgtatttgcctttattcctcacatcaaagggatattatattaatttcttatgtttgccgggaaaacaaagcaacttcc

SEQ ID NO: 73 - protein, 437 aa, Mus musculus
  ref <308>: DATABASE ACCESSION NUMBER: EMBL Accession Nos. AJ245857; CAC80975.1
  ref <309>: DATABASE ENTRY DATE: 2003-02-01
  ref <313>: RELEVANT RESIDUES IN SEQ ID NO: (1)..(437)
MASLGPSPWAPLSTPAPTAQLLLFLLLQVSAQPQGLSGMQGEPSLGDSSSGEDELGVDVLPSEEDAPEEADPPDGEDPPEVNSEDRMEESLGLEDLSTPEAPEHSQGSHGDEKGGGHSHWSYGGTLLWPQVSPACAGRFQSPVDIRLERTAFCRTLQPLELLGYELQPLPELSLSNNGHTVQLTLPPGLKMALGPGQEYRALQLHLHWGTSDHPGSEHTVNGHRFPAEIHVVHLSTAFSELHEALGRPGGLAVLAAFLQESPEENSAYEQLLSHLEEISEEGSKIEIPGLDVSALLPSDLSRYYRYEGSLTTPPCSQGVIWTVFNETVKLSAKQLHTLSVSLWGPRDSRLQLNFRATQPLNGRTIEASFPAAEDSSPEPVHVNSCFTAGDILALVFGLLFAVTSIAFLLQLRRQHRHRSGTKDRVSYSPAEMTETGA

SEQ ID NO: 74 - protein, 31 aa, Mus musculus
MASLGPSPWAPLSTPAPTAQLLLFLLLQVSA

SEQ ID NO: 75 - protein, 358 aa, Mus musculus
QPQGLSGMQGEPSLGDSSSGEDELGVDVLPSEEDAPEEADPPDGEDPPEVNSEDRMEESLGLEDLSTPEAPEHSQGSHGDEKGGGHSHWSYGGTLLWPQVSPACAGRFQSPVDIRLERTAFCRTLQPLELLGYELQPLPELSLSNNGHTVQLTLPPGLKMALGPGQEYRALQLHLHWGTSDHPGSEHTVNGHRFPAEIHVVHLSTAFSELHEALGRPGGLAVLAAFLQESPEENSAYEQLLSHLEEISEEGSKIEIPGLDVSALLPSDLSRYYRYEGSLTTPPCSQGVIWTVFNETVKLSAKQLHTLSVSLWGPRDSRLQLNFRATQPLNGRTIEASFPAAEDSSPEPVHVNSCFTAG

SEQ ID NO: 76 - protein, 60 aa, Mus musculus
SSSGEDELGVDVLPSEEDAPEEADPPDGEDPPEVNSEDRMEESLGLEDLSTPEAPEHSQG

SEQ ID NO: 77 - protein, 258 aa, Mus musculus
EKGGGHSHWSYGGTLLWPQVSPACAGRFQSPVDIRLERTAFCRTLQPLELLGYELQPLPELSLSNNGHTVQLTLPPGLKMALGPGQEYRALQLHLHWGTSDHPGSEHTVNGHRFPAEIHVVHLSTAFSELHEALGRPGGLAVLAAFLQESPEENSAYEQLLSHLEEISEEGSKIEIPGLDVSALLPSDLSRYYRYEGSLTTPPCSQGVIWTVFNETVKLSAKQLHTLSVSLWGPRDSRLQLNFRATQPLNGRTIEASF

SEQ ID NO: 78 - protein, 48 aa, Mus musculus
DILALVFGLLFAVTSIAFLLQLRRQHRHRSGTKDRVSYSPAEMTETGA

SEQ ID NO: 79 - protein, 22 aa, Mus musculus
DILALVFGLLFAVTSIAFLLQL

SEQ ID NO: 80 - protein, 26 aa, Mus musculus
RRQHRHRSGTKDRVSYSPAEMTETGA

SEQ ID NO: 81 - protein, 74 aa, Homo sapiens
GSSGEDDPLGEEDLPSEEDSPREEDPPGEEDLPGEEDLPGEEDLPEVKPKSEEEGSLKLEDLPTVEAPGDPQEP

SEQ ID NO: 82 - protein, 277 aa, Homo sapiens
DPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSS

SEQ ID NO: 83 - protein, 346 aa, Homo sapiens
GSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSS

SEQ ID NO: 84 - protein, 32 aa, Homo sapiens
GDDQSHWRYGGDPPWPRVSPACAGRFQSPVDI

SEQ ID NO: 85 - DNA, 21 bp, Mus musculus
gttggcatagaggtctttacg

SEQ ID NO: 86 - DNA, 20 bp, Mus musculus
gccgcatcctcttcctccct

SEQ ID NO: 87 - DNA, 21 bp, Mus musculus
ggaggcctggcagttttggct

SEQ ID NO: 88 - DNA, 23 bp, Mus musculus
ctccagtttctgtcatctctgcc

SEQ ID NO: 89 - DNA, 20 bp, Mus musculus
ccctgctgcagaggatagca

SEQ ID NO: 90 - DNA, 20 bp, Mus musculus
ggtcccacttctgtgcctgt

SEQ ID NO: 91 - DNA, 21 bp, Mus musculus
ctcggaggaagtccatgtgaa

SEQ ID NO: 92 - DNA, 20 bp, Mus musculus
ggtcccacttctgtgcctgt

SEQ ID NO: 93 - DNA, 20 bp, Homo sapiens
ccaaccgcgggaagatgacc

SEQ ID NO: 94 - DNA, 21 bp, Homo sapiens
gatcttcatgaggtagtcagt

SEQ ID NO: 95 - DNA, 22 bp, Homo sapiens
gaaccccagaataatgcccaca

SEQ ID NO: 96 - DNA, 22 bp, Homo sapiens
tcgcttggaagaaatcgctgag

SEQ ID NO: 97 - DNA, 23 bp, Homo sapiens
gttgctgtctcgcttggaagaaa

SEQ ID NO: 98 - DNA, 21 bp, Homo sapiens
gcggtagctcacacccccttt

SEQ ID NO: 99 - DNA, 21 bp, Homo sapiens
tatctgcactcctgccctctg

SEQ ID NO: 100 - DNA, 22 bp, Homo sapiens
cacagggtgtcagagagggtgt

SEQ ID NO: 101 - DNA, 25 bp, Homo sapiens
ctaggatgtcacctgcttagcactc

SEQ ID NO: 102 - DNA, 24 bp, Mus musculus
tccatgtgaattcctgcttcactg

SEQ ID NO: 103 - DNA, 24 bp, Mus musculus
cttcctccgagatttcttccaaat

SEQ ID NO: 104 - DNA, 23 bp, Homo sapiens
gtgacatcctagccctggttttt

SEQ ID NO: 105 - DNA, 23 bp, Homo sapiens
ctgcttagcactcagcatcactg

SEQ ID NO: 106 - DNA, 1671 bp, Mus musculus
  CDS (33)..(1052)
acagtgtgtcctgggacaccccagtcagctgcatggcctccctgggccccagcccctgggcccctttgtcgaccccagcccctactgcacagctgctgctgttcctgctgctccaagtgtctgctcagccccagggcctgtccgggatgcagggggagccctccttgggagacagttcatctggggaggatgagctgggcgtggatgttctgcccagtgaagaggatgcacctgaagaggcggatccacccgatggggaggatccacctgaagttaattctgaagacaggatggaggagtcccttgggttagaggatctatcgactcccgaggctcctgagcacagtcaaggttcccacggggatgaaaaagggggtggccacagtcattggagctatggaggcaccctactctggccccaggtgtccccagcctgtgctggccgctttcagtccccggtagacatccgcctggagcgcaccgcattctgccgcactctgcaacccctggaacttctgggttatgagctccagcctctcccggaactgagcctatccaacaatggccacacggtacagctgactctgccgccggggctgaagatggctctgggacctgggcaggaataccgggccttgcagttgcatctacactggggaacttcagatcacccaggctcagaacacacagtcaatggtcaccgtttccctgctgagatccacgtggtgcacctcagtactgctttctccgaacttcatgaagccttgggccgcccaggaggcctggcagttttggctgcctttctgcaggagagcccagaagaaaacagtgcttacgaacagttgctgtcccatttggaagaaatctcggaggaagtccatgtgaattcctgcttcactgctggtgacatcctagccttggtgtttggccttctctttgctgtcaccagcatcgccttcctcctacagctgaggaggcagcacaggcacagaagtgggaccaaagatagagttagctacagtccggcagagatgacagaaactggagcctaataaggagaaaccagctaaaggaatctggaaaccttgtcctgtcctgcctgttatacagcctcctttttaaccactacgaaatcatttccatctttttaactttcttctttccttaaagtactgtgataggagctaaccgtgaaaaaacaagattgggctcaattcttgctgggttttgatggttttgaagacagggtttctctgtgtagccctggctgtcctgggactcactctgtagcctaggctggccttgaactcagagatccgattgcctctgccttcttagtgccaggactgagggtatgtaccactaccaccgccaccccgcaattcttgcttttattctgcagtcaaatgctccaccactgagctacacacccctccatcttactatgcaggcagactggccttgcattgtgtatgtatggtgatggagtttgaagccagggtcttacctactaggcaagcgttctatcactgacctaagcctctctagtcccttctcttgaaccctctattctcctgtcccagcctcttgaatgctgggattacaggaatgcaccatcatgcttggtcatcccaattatttaaaaaaaatatataaatcttcctagtaaaa

SEQ ID NO: 107 - protein, 339 aa, Mus musculus
MASLGPSPWAPLSTPAPTAQLLLFLLLQVSAQPQGLSGMQGEPSLGDSSSGEDELGVDVLPSEEDAPEEADPPDGEDPPEVNSEDRMEESLGLEDLSTPEAPEHSQGSHGDEKGGGHSHWSYGGTLLWPQVSPACAGRFQSPVDIRLERTAFCRTLQPLELLGYELQPLPELSLSNNGHTVQLTLPPGLKMALGPGQEYRALQLHLHWGTSDHPGSEHTVNGHRFPAEIHVVHLSTAFSELHEALGRPGGLAVLAAFLQESPEENSAYEQLLSHLEEISEEVHVNSCFTAGDILALVFGLLFAVTSIAFLLQLRRQHRHRSGTKDRVSYSPAEMTETGA

SEQ ID NO: 108 - DNA, 1380 bp, Homo sapiens
  CDS (43)..(1119)
gcccgtacacaccgtgtgctgggacaccccacagtcagccgcatggctcccctgtgccccagcccctggctccctctgttgatcccggcccctgctccaggcctcactgtgcaactgctgctgtcactgctgcttctgatgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcaggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataat

SEQ ID NO: 109 - protein, 358 aa, Homo sapiens
MAPLCPSPWLPLLIPAPAPGLTVQLLLSLLLLMPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQVTS

(19) United States
(12) Patent Application Publication
(10) Pub. No.: US 2010/0056611 A1
(43) Pub. Date: Mar. 4, 2010

(54) MN/CA9 Splice Variants

(76) Inventors: Jaromir Pastorek, Stupava, (SK); Monika Barathova, Bratislava (SK)

Correspondence Address: LEONA L. LAUDER, 235 MONTGOMERY STREET, SUITE 1026, SAN FRANCISCO, CA, 94104-0332, US

(21) Appl. No.: 12/444,888

(22) PCT Filed: Oct. 12, 2007
(86) PCT No.: PCT/US2007/021905

§ 371 Date: Oct. 14, 2009

Related U.S. Application Data
(60) Provisional application No. 60/851,398, filed on Oct. 12, 2006.

(51) Int. Cl. A61K 31/7088; C12Q 1/68; C07H 21/04; C07K 16/00

(52) U.S. Cl. 514/44 R; 435/6; 536/24.31; 536/24.33; 536/23.1; 530/387.1

(57) ABSTRACT

Herein disclosed is an alternatively-spliced [AS] variant of MN/CA9 mRNA and its related protein—AS MN/CA IX. Unlike the tumor-associated, full-length [FL] MN/CA9 mRNA and FL MN/CA IX, which in most tissues signify oncogenesis and/or hypoxia, the AS MN/CA9 mRNA is constitutively-expressed under normoxia and is not stimulated by hypoxia, and the AS MN/CA IX is not confined to the cell membrane. Provided herein are diagnostic/prognostic methods for preneoplastic/neoplastic disease to differentiate between AS and FL MN/CA9 expression, and probes, primers, and antibodies useful in such methods. Also disclosed are methods to treat pre-neoplastic/neoplastic disease involving the MN gene and protein, which methods are based on the ability of AS MN protein (AS MN/CA IX) to interfere with the catalytic activity of FL MN protein (FL MN/CA IX); such methods may also use AS MN protein fragments that have that interference capability. Such methods may comprise increasing the levels of AS MN/CA IX relative to the levels of FL MN/CA IX. Exemplary therapeutic methods may comprise the administration of agents, such as, AS MN/CA IX itself, a vector expressing AS MN/CA9 mRNA, an antisense oligonucleotide that blocks expression of FL MN/CA IX but not that of AS MN/CA IX, a vector expressing such an antisense oligonucleotide, a FL MN/CA9 isoform-specific siRNA, or a vector expressing such FL MN/CA9 isoform-specific siRNA. Further disclosed are methods to identify agents capable of modulating levels of AS MN/CA IX.

### FIELD OF THE INVENTION

The present invention is in the general area of medical genetics and in the fields of biochemical engineering, immunochemistry and oncology. More specifically, it relates to the MN gene—a cellular gene considered to be an oncogene, known alternatively as MN/CA9, CA9, or carbonic anhydrase 9, which gene encodes the oncoprotein now known alternatively as the MN protein, the MN/CA IX isoenzyme, MN/CA IX, carbonic anhydrase IX, CA IX or the MN/G250 protein.

More specifically, the instant invention is directed to an alternatively-spliced [AS] form of MN/CA9 mRNA, and probes/primers to detect it. The AS MN/CA9 mRNA is primarily expressed in normal cells and under normoxia, and can interfere with assays measuring the expression of full-length [FL] MN/CA9 mRNA, particularly in RT-PCR assays. This invention is also directed to the AS form of MN/CA IX, and diagnostic/prognostic methods using assays to detect or to detect and quantify it, alone or in combination with the FL form of MN/CA IX protein. Further, this invention concerns therapeutic methods exploiting MN/CA9 alternative splicing as a means to target FL MN/CA IX protein. The forms of AS MN/CA9/CA IX that are the focus of this invention can be vertebrate, but preferably mammalian, and more preferably human.

### BACKGROUND OF THE INVENTION

As indicated above, the MN gene and protein are known by a number of alternative names, which names are used herein interchangeably. The MN protein was found to bind zinc and have carbonic anhydrase (CA) activity and is now considered to be the ninth carbonic anhydrase isoenzyme—MN/CA IX or CA IX [Opavsky et al. (1996), infra]. According to the carbonic anhydrase nomenclature, human CA isoenzymes are written in capital roman letters and numbers, while their genes are written in italic letters and arabic numbers. Alternatively, “MN” is used herein to refer either to carbonic anhydrase isoenzyme IX (CA IX) proteins/polypeptides, or carbonic anhydrase isoenzyme 9 (CA9) gene, nucleic acids, cDNA, mRNA etc. as indicated by the context.

The MN protein has also been identified with the G250 antigen. Uemura et al., “Expression of Tumor-Associated Antigen MN/G250 in Urologic Carcinoma: Potential Therapeutic Target, “J. Urol., 154 (4 Suppl.): 377 (Abstract 1475; 1997) states: “Sequence analysis and database searching revealed that G250 antigen is identical to MN, a human tumor-associated antigen identified in cervical carcinoma (Pastorek et al.,1994).”

CA IX is a cancer-related carbonic anhydrase identified by Zavada, J., Pastorekova, S. and Pastorek, J. [“Zavada et al.,” see, for example, U.S. Pat. No. 5,387,676] using the M75 monoclonal antibody first described by Pastorekova et al. [Virology 187: 620-626 (1992)]. That antibody was employed in cloning of cDNA encoding CA IX [Pastorek et al., Oncogene, 9: 2788-2888 (1994)], in the assessment of CA IX expression in tumors and normal tissues [Zavada et al., Int J Cancer. 54: 268-274, (1993), and many other references], in the study of CA IX regulation by cell density [Lieskovska et al., Neoplasma. 46: 17-24, (1999), Kaluz et al., Cancer Research, 62: 4469-4477, (2002)] as well in demonstration of CA IX induction by hypoxia [Wykoff et al., Cancer Research, 60: 7075-7083 (2000), and many other references]. All such studies supported Zavada et al's original conception and work [for example, Zavada et al., U.S. Pat. No. 5,387,676] that MN/CA IX/CA9 can be used diagnostically and/or prognostically as a preneoplastic/neoplastic tumor marker and therapeutically as a target, and showed that the M75 monoclonal antibody is a valuable CA IX-specific reagent useful for different immunodetection methods and immunotargeting approaches.

Zavada et al., International Publication Number WO 93/18152 (published 16 Sep. 1993) and U.S. Pat. No. 5,387,676 (issued Feb. 7, 1995), describe the discovery and biological and molecular nature of the MN gene and protein. The MN gene was found to be present in the chromosomal DNA of all vertebrates tested, and its expression to be strongly correlated with tumorigenicity.

The MN protein was first identified in HeLa cells, derived from a human carcinoma of cervix uteri. It is found in many types of human carcinomas (notably uterine cervical, ovarian, endometrial, renal, bladder, breast, colorectal, lung, esophageal, and prostate, among others). Very few normal tissues have been found to express MN protein to any significant degree. Those MN-expressing normal tissues include the human gastric mucosa and gallbladder epithelium, and some other normal tissues of the alimentary tract. Paradoxically, MN gene expression has been found to be lost or reduced in carcinomas and other preneoplastic/neoplastic diseases in some tissues that normally express MN, e.g., gastric mucosa.

In general, oncogenesis may be signified by the abnormal expression of MN protein. For example, oncogenesis may be signified: (1) when MN protein is present in a tissue which normally does not express MN protein to any significant degree; (2) when MN protein is absent from a tissue that normally expresses it; (3) when MN gene expression is at a significantly increased level, or at a significantly reduced level from that normally expressed in a tissue; or (4) when MN protein is expressed in an abnormal location within a cell.

Zavada et al., WO 93/18152 and Zavada et al., WO 95/34650 (published 21 Dec. 1995) disclose how the discovery of the MN gene and protein and the strong association of MN gene expression and tumorigenicity led to the creation of methods that are both diagnostic/prognostic and therapeutic for cancer and precancerous conditions. Methods and compositions were provided therein for identifying the onset and presence of neoplastic disease by detecting or detecting and quantitating abnormal MN gene expression in vertebrates. Abnormal MN gene expression can be detected or detected and quantitated by a variety of conventional assays in vertebrate samples, for example, by immunoassays using MN-specific antibodies to detect or detect and quantitate MN antigen, by hybridization assays or by PCR assays, such as RT-PCR, using MN nucleic acids, such as, MN cDNA, to detect or detect and quantitate MN nucleic acids, such as, MN mRNA.

MN/CA IX was first identified in HeLa cells, as both a plasma membrane and nuclear protein with an apparent molecular weight of 58 and 54 kilodaltons (kDa) as estimated by Western blotting. It is N-glycosylated with a single 3 kDa carbohydrate chain and under non-reducing conditions forms S-S-linked oligomers [Pastorekova et al., Virology, 187: 620-626 (1992); Pastorek et al., Oncogene, 9: 2788-2888 (1994)]. MN/CA IX is a transmembrane protein located at the cell surface, although in some cases it has been detected in the nucleus [Zavada et al., Int. J. Cancer. 54: 268-274 (1993); Pastorekova et al., supra].

MN is manifested in HeLa cells by a twin protein, p54/58N. Immunoblots using a monoclonal antibody reactive with p54/58N (MAb M75) revealed two bands at 54 kd and 58 kd. Those two bands may correspond to one type of protein that most probably differs by post-translational processing.

Zavada et al., WO 93/18152 and/or WO 95134650 disclose the MN cDNA sequence (SEQ ID NO: 1) shown herein in FIG. 8A-8C, the MN amino acid sequence (SEQ ID NO: 2) also shown in FIG. 8A-8C, and the MN genomic sequence (SEQ ID NO: 3) shown herein in FIG. 9A-9F. The MN gene is organized into 11 exons and 10 introns. The human MN cDNA sequence of SEQ ID NO: 1 contains 1522 base pairs (bp). The MN cDNA sequence of SEQ ID NO: 70 contains 1552 bp [EMBL Acc. No. X66839; Pastorek et al. (1994)].

The first thirty seven amino acids of the MN protein shown in FIG. 8A-8C (SEQ ID NO: 2) constitute the putative MN signal peptide [SEQ ID NO: 4]. The MN protein has an extracellular (EC) domain [amino acids (aa) 38-414 of FIG. 8A-8C (SEQ ID NO: 5)], a transmembrane (TM) domain [aa 415-434 (SEQ ID NO: 6)] and an intracellular (IC) domain [aa 435-459 (SEQ ID NO: 7)]. The extracellular domain contains the proteoglycan-like (PG) domain at about amino acids (aa) 53-111 (SEQ ID NO. 8) or preferably at about aa 52-125 (SEQ ID NO: 81), and the carbonic anhydrase (CA) domain at about aa 135-391 (SEQ ID NO: 9) or preferably, at about aa 121-397 (SEQ ID NO: 82).

Zavada et al, WO 93/18152 and WO 95/34650 describe the production of MN-specific antibodies. A representative and preferred MN-specific antibody, the monoclonal antibody M75 (Mab M75), the hybridoma for which (VU-M75) was deposited at the American Type Culture Collection (ATCC) in Manassas, Va. (USA) under ATCC Number HB 11128. The M75 antibody was used to discover and identify the MN protein and can be used to identify readily MN antigen in Western blots, in radioimmunoassays and immunohistochemically, for example, in tissue samples that are fresh, frozen, or formalin-, alcohol-, acetone- or otherwise fixed and/or paraffin-embedded and deparaffinized. Another representative and preferred MN-specific antibody, Mab MN12, is secreted by the hybridoma MN 12.2.2, which was deposited at the ATCC under the designation HB 11647. Example 1 of Zavada et al., WO 95/34650 provides representative results from immunohistochemical staining of tissues using MAb M75, which results demonstrate the MN gene's oncogenicity.

Immunodominant epitopes are considered to be essentially those that are within the PG domain of MN/CA IX, including the repetitive epitopes for the M75 mab, particularly the amino acid sequence PGEEDLP (SEQ ID NO: 11), which is 4× identically repeated in the N-terminal PG region [Zavada et al. (2000), infra]. The epitope for the MN12 mab is also immunodominant.

The M75 mab was first reported in Pastorekova et al., Virology, 187: 620-626 (1992) and is claimed specifically, as well as generically with all MN/CA IX-specific antibodies, polyclonal and monoclonal as well as fragments thereof, in a number of U.S. and foreign patents, including, for example, Zavada et al., U.S. Pat. No. 5,981,711 and EP 0 637 336 B1. [See also, Zavada et al., U.S. Pat. Nos. 5,387,676; 5,955,075; 5,972,353; 5,989,838; 6,004,535; 6,051,226; 6,069,242; 6,093,548; 6,204,370; 6,204,887; 6,297,041; and 6,297,051; and Zavada et al., AU 669694; CA 2,131,826; DE 69325577.3; and KR 282284.] Those Zavada et al. U.S. and foreign patents are herein incorporated by reference.

CA IX is a highly active member of a carbonic anhydrase family of zinc metalloenzymes that catalyze the reversible conversion between carbon dioxide and bicarbonate [Pastorek et al. (1994); Opavsky et al. (1996); Chegwidden et al., (2000), infra; Wingo et al, (2001), infra; Pastorekova et al. (2004), infra]. It is one of 14 isoforms that exist in mammals and occupy different subcellular positions, including cytoplasm (CA I, II, III, VII), mitochondrion (CA VA, VB), secretory vesicles (CA VI) and plasma membrane (CA IV, IX, XIl, XIV). Some of the isozymes are distributed over broad range of tissues (CA I, II, CA IV), others are more restricted to particular organs (CA VI in salivary glands) and two isoforms have been linked to cancer tissues (CA IX, XII) [reviewed in Chegwidden (2000); Pastorekova and Pastorek, Chapter 9, Carbonic Anhydrase: Its Inhibitors and Activators (eds. Supuran et al.; CRC Press (London et al.) 2004]. Enzyme activity and kinetic properties, as well as sensitivity to sulfonamide inhibitors vary from high (CA II, CA IX, CA XII, CA IV) to low (CA III) [Supuran and Scozzafava (2000), infra]. Several isoforms designated as CA-related proteins (CA-RP VIII, X, XI) are acatalytic due to incompletely conserved active site. This extraordinary variability among the genetically related members of the same family of proteins creates a basis for their employment in diverse physiological and pathological processes. The catalytic activity is of fundamental relevance for the maintenance of acid-base balance and exchange of ions and water in metabolically active tissues. Via this activity, CAs substantially contribute to respiration, production of body fluids (vitreous humor, gastric juice, cerebrospinal fluid), bone resorption, renal acidification etc. (Chegwidden et al. 2000).

CA IX isozyme integrates several properties that make it an important subject of basic as well as clinical research. First of all, expression of CA IX is very tightly associated with a broad variety of human tumors, while it is generally absent from the corresponding normal tissues [Zavada et al. (1993); Liao et al. (1994), infra; Turner et al. 1997, infra; Liao et al. 1997, infra; Saarnio et al., 1998, infra; Vermylen et al., 1999, infra; Ivanov et al. (2001), infra; Bartosova et al. (2002), infra]. This is principally related to tumor hypoxia that strongly activates transcription of the CA9 gene via a hypoxia-inducible factor (HIF), which binds to a hypoxia responsive element (HRE) localized in the minimal CA9 promoter proximal to transcription start site at the −10/−3 position [Wykoff et al. (2000), infra]. The HIF transcription factor significantly changes the expression profile of weakly oxygenated tumor cells by activation of genes that either support their survival and adaptation to hypoxic stress or lead to their death. As a result, hypoxia selects for more aggressive tumor cells with increased capability to invade and metastasize and is therefore inherently associated with bad prognosis and poor response to anticancer therapy [Harris (2002), infra].

Since tumor hypoxia is an important phenomenon with dramatic implications for cancer development and therapy [Hockel and Vaupel (2001), infra], MN bears a significant potential as an intrinsic hypoxic marker with a prognostic/predictive value and as a promising therapeutic target [Wykoff et al. (2000); Wykoff et al. (2001), infra; Beasley et al. (2001), infra; Giatromanolaki et al. (2001), infra; Koukourakis et al. (2001), infra; Potter and Harris (2003), infra]. In favor of the proposed clinical applications, CA IX is an integral plasma membrane protein with a large extracellular part exposed at the surface of cancer cells and is thus accessible by the targeting tools, including the specific monoclonal antibodies. Furthermore, CA IX differs from the other CA isozymes by the presence of a unique proteoglycan-related region (PG) that forms an N-terminal extension of the extracellular CA domain and allows for elimination of cross-recognition with other isozymes [Opavsky et al. (1996)]. CA IX appears to play an active role in tumor biology both via modulation of cell adhesion and control of pH (Svastova et al, 2003, infra, Svastova et al, 2004, infra, Swietach et al, 2007, infra). CA IX participates in bicarbonate transport metabolon and contributes to acidification of extracellular microenvironment in response to hypoxia (Morgan et al, 2007, infra, Svastova et al, 2004, infra). In addition, CA IX's intracellular domain (IC) has a potential third tumorigenic role, at least in renal cell carcinoma: tyrosine-phosphorylated CA IX (mediated via EGFR) interacts with the regulatory subunit of PI-3K (p85), resulting in activation of Akt [Dorai et al. (2005), infra]. Because of its many potential activities contributing to oncogenesis, targeting the CA IX protein for abrogation of its function is expected to have therapeutic effects. However, many basic molecular and functional aspects of CA IX have been unknown; one of which had been CA IX's potential alternative splicing.

Alternative splicing is an important molecular mechanism that contributes to structural and functional diversification of proteins. It frequently results from differential exon inclusion and leads to altered domain composition, subcellular localization, interaction potential, signalling capacity and other changes at the protein level. Data obtained by recent genomic technologies indicate that over 60% of human genes are alternatively spliced. It is also becoming increasingly evident that imbalances in expression of alternative splicing variants can significantly affect cell phenotype and play a role in various pathologies (Matlin et al, 2005, infra).

The instant invention is based upon the discovery that CA9 gene expression involves alternative splicing. Herein are described alternatively spliced (AS) mouse and human variants of MN mRNA. The inventors demonstrate that the human AS variant is less abundant than the full-length (FL) CA9 mRNA in tumors, but can be detected in normal tissues and under normoxia. The human AS CA9 mRNA does not contain exons 8 and 9 and codes for a truncated CA IX protein. Consequently, the AS CA IX is not confined to plasma membrane and shows reduced catalytic activity. Upon overexpression in HeLa cells, the AS CA IX reduces hypoxia-induced extracellular acidification and compromises growth of HeLa spheroids. Because the AS variant can be present in normoxic cells with a normal phenotype, it can produce false-positive results in diagnostic and/or prognostic studies designed to assess hypoxia- and tumor-related expression of CA9 gene. Moreover, the AS form of CA IX protein may functionally interfere with the FL CA IX form, especially under moderate hypoxia, when the FL levels are relatively low.

### SUMMARY OF THE INVENTION

The invention is based on the discovery that, in addition to a full-length (FL) CA9 mRNA transcript encoding a hypoxia-induced, membrane-bound and tumor-associated MN protein, there is also a constitutively-produced, alternatively-spliced (AS) CA9 mRNA transcript that encodes an AS MN protein (AS MN/CA IX or AS CA IX) which is not confined to the plasma membrane. A further discovery, upon which therapeutic aspects of the invention are based, is that AS CA IX interferes with the function of the FL CA IX. The hypoxia- and tumor-independent production of the AS variant of CA IX has many implications for diagnostic, prognostic and therapeutic aspects of CA IX.

This invention in one aspect concerns diagnostic and/or prognostic methods for preneoplastic/neoplastic diseases associated with abnormal MN/CA IX expression in vertebrates, preferably mammals, more preferably in humans, comprising differentiating between full-length [FL] and alternatively-spliced [AS] MN/CA9 mRNA or AS and FL MN/CA IX expression.

Said methods may comprise the use of one or more probes and/or primers to detect or detect and quantitate FL and/or AS MN/CA9 mRNA expression; preferably, said methods comprise the use of: (a) probes and/or primers to detect full-length [FL] MN/CA9 mRNA but not alternatively-spliced [AS] MN/CA9 mRNA; (b) probes and/or primers to detect AS MN/CA9 mRNA but not FL MN/CA9 mRNA; and/or (c) probes and/or primers to detect both FL and AS MN/CA9 mRNA. The diagnostic/prognostic methods of the invention in one aspect exploit the differences between the alternatively spliced MN/CA9 nucleic acids and the full length MN/CA9 nucleic acids, for example, by targeting probes to a splice junction present in AS MN/CA9 mRNA but not in FL MN/CA9 mRNA, or to a nucleic acid sequence absent in AS MN/CA9 mRNA but present in FL MN/CA9 mRNA. Analogously, primer pairs can be designed, for example, to amplify regions found only in AS MN/CA9 mRNA and not in FL MN/CA9 mRNA or vice versa. Ones of skill in the art in view of the instant disclosure would be able to design any number of probes and/or primers/primer pairs that would be useful in the diagnostic/prognostic methods of this invention.

In a preferred diagnostic/prognostic method for preneoplastic/neoplastic diseases in humans, one or more particularly preferred probes and/or primers of the invention is/are selected from the group consisting of SEQ ID NOS: 97-101 and nucleic acid sequences that are at least 80% homologous to SEQ ID NOS: 97-101, more preferably at least 90% homologous to SEQ ID NOS: 97-101. Said methods comprising the use of one or more probes and/or primers to detect or detect and quantitate FL and/or AS MN/CA9 mRNA expression, may further comprise determining the ratio of FL:AS MN/CA9 mRNA, or changes in the ratio of FL:AS MN/CA9 mRNA over time.

Further, said AS MN/CA9 mRNA expression can be used to indicate normal MN/CA9 gene expression, and said FL MN/CA9 mRNA expression to indicate abnormal MN/CA9 gene expression, particularly the levels of said AS and/or FL MN/CA9 mRNA expression. Alternatively, or additionally, said AS MN/CA9 mRNA expression can be used to indicate normoxic MN/CA9 gene expression, and said FL MN/CA9 mRNA expression to indicate hypoxic MN/CA9 gene expression. Again, the levels of said MN AS and/or FL mRNA expression would be of particular indicative value.

Said methods comprising the use of one or more probes and/or primers, to detect or detect and quantitate FL and/or AS MN/CA9 mRNA expression, may further comprise the use of a nucleic acid amplification method, preferably an amplification method selected from PCR, RT-PCR, real-time PCR or quantitative real-time RT-PCR and equivalent nucleic acid amplification methods known to those of skill in the art. Alternatively, said methods to detect or detect and quantitate FL and/or AS MN/CA9 mRNA expression may comprise the use of a microarray chip. For example, said microarray chip may comprise a probe that binds to full-length [FL] MN/CA9 mRNA but not to alternatively-spliced [AS] MN/CA9 mRNA, and/or a probe that binds to AS MN/CA9 mRNA but not FL MN/CA9 mRNA, wherein strategically locating said probe(s) on such a chip is within the skill of the art.

In another aspect, the invention concerns diagnostic and/or prognostic methods for preneoplastic/neoplastic diseases associated with abnormal MN/CA IX expression in a mammal, comprising differentiating between FL and AS MN/CA IX expression. Preferably, said methods comprise the use of one or more antibodies to differentiate between FL and AS MN/CA IX expression in a preneoplastic/neoplastic tissue. Said methods may comprise detecting or detecting and quantitating AS MN/CA IX in said tissue; and may further comprise determining the ratio of FL MN/CA IX levels to AS MN/CA IX levels in said tissue. Further, said FL:AS MN/CA IX ratio may be used to indicate the presence or degree of hypoxia in said tissue.

In one preferred embodiment of the invention, the diagnostic and/prognostic methods comprise detecting or detecting and quantitating FL MN/CA IX and AS MN/CA IX in a vertebrate tissue, comprising the steps of:
(a) contacting said sample synchronously or sequentially with at least two antibodies, at least two antigen binding antibody fragments, or a mixture of antibodies and antigen-binding antibody fragments, wherein at least one antibody/antibody fragment specifically binds to FL MN/CA IX protein but not to AS MN/CA IX protein, and wherein at least one other antibody/antibody fragment specifically binds to both FL and AS MN/CA IX;
(b) detecting and quantifying the binding of said antibodies/antibody fragments in said sample; and
(c) comparing the binding of said differentially binding antibodies/antibody fragments to determine the relative levels of FL MN/CA IX and AS MN/CA IX.

Preferably, said antibody/antibody fragment, or antibodies/antibody fragments, that specifically bind(s) to FL MN/CA IX but not to AS MN/CA IX is/are specific for the carbonic anhydrase (CA) domain of MN/CA IX; and said antibody/antibody fragment, or antibodies/antibody fragments, that specifically bind(s) both FL MN/CA IX and AS MN/CA IX is/are specific for the proteoglycan-like (PG) domain of MN/CA IX. Still more preferably, said antibody specific for the CA domain of MN/CA IX is the V/10 monoclonal antibody which is produced by the hybridoma VU-V/10, deposited at BCCM™/LMBP in Ghent, Belgium under Accession No. LMBP 6009CB; and said antibody specific for the PG domain of MN/CA IX is the M75 monoclonal antibody which is produced by the hybridoma VU-M75 deposited at the American Type Culture Collection (ATCC) under the ATCC designation No. HB 11128.

Still further, the invention is directed to diagnostic and/or prognostic methods for preneoplastic/neoplastic diseases associated with abnormal MN/CA IX expression in a vertebrate, comprising detecting or detecting and quantitating full-length [FL] MN/CA IX protein but not alternatively-spliced [AS] MN/CA IX protein in an appropriate vertebrate tissue sample, comprising the steps of:
(a) contacting said sample with an antibody or antibody fragment, wherein said antibody or antibody fragment specifically binds to FL MN/CA IX but not to AS MN/CA IX; and
(b) detecting and quantifying binding of said antibody/antibody fragment in said sample.
   Said vertebrate is preferably a mammal, and said mammal is more preferably a human.

An exemplary and preferred antibody or antibody fragment, which specifically binds to FL MN/CA IX but not to AS MN/CA IX, is one which is specific for the carbonic anhydrase (CA) domain of MN/CA IX. More preferably, said antibody specific for the CA domain of MN/CA IX is the V/10 monoclonal antibody which is produced by the hybridoma VU-V/10, deposited at BCCM™/LMBP in Ghent, Belgium under Accession No. LMBP 6009CB.

A particularly preferred embodiment of the invention concerns diagnostic and/or prognostic methods for preneoplastic/neoplastic diseases associated with abnormal MN/CA IX expression in a vertebrate, preferably a mammal, comprising detecting or detecting and quantitating full-length [FL] MN/CA9 mRNA but not alternatively-spliced [AS] MN/CA9 mRNA in a vertebrate, preferably mammalian preneoplastic/neoplastic sample, comprising contacting mRNA from said sample with a primer or a probe that specifically binds to FL MN/CA9 mRNA but not to AS MN/CA9 mRNA.

The invention further concerns nucleic acid probes and/or primers which are used to differentiate between alternatively-spliced [AS] MN/CA9 mRNA and full-length [FL] MN/CA9 mRNA expression in a mammal. The design of such probes/primers based upon the instant disclosure, as noted above, is within the skill of the art. Preferably, wherein said mammal is a human, and said probe and/or primer is used to detect AS MN/CA9 mRNA but not FL MN/CA9 mRNA, said probe or primer comprises a nucleic acid which binds to the splice junction of exons 7 and 10 of the MN/CA9 gene. More preferably, said probe or primer has a sequence of SEQ ID NO: 101 or a sequence that is at least 80% homologous to SEQ ID NO: 101, more preferably at least 90% homologous to SEQ ID NO: 101. Alternatively, wherein said mammal is a human, and said probe or primer is used to detect FL MN/CA9 mRNA but not AS MN/CA9 mRNA, said probe or primer comprises a nucleic acid which binds to exon 8 or exon 9 of the human MN/CA9 gene, or binds to the splice junction of exons 7 and 8, the splice junction of exons 8 and 9, or the splice junction of exons 9 and 10 of the human MN/CA9 gene. More preferably, said probe or primer used to detect human FL MN/CA9 mRNA but not AS MN/CA9 mRNA has a sequence of SEQ ID NO: 100 or a sequence that is at least 80% homologous to SEQ ID NO: 100, more preferably at least 90% homologous to SEQ ID NO: 100. The invention further relates to a vector that expresses such a probe or primer, and/or a host cell comprising such a vector, and to a microarray chip comprising one or more such probes.

The invention further concerns a pair of probes and/or primers used to differentiate between alternatively-spliced [AS] MN/CA9 mRNA and full-length [FL] MN/CA9 mRNA expression in a mammal. Said pair of probes and/or primers can be used to detect alternatively-spliced [AS] human MN/CA9 mRNA but not full-length [FL] human MN/CA9 mRNA. An exemplary and preferred pair of probes or primers used to detect alternatively-spliced [AS] human MN/CA9 mRNA but not full-length [FL] human MN/CA9 mRNA consists of SEQ ID NOS: 99 and 101, or nucleic acid sequences that are at least 80% homologous, more preferably at least 90% homologous to SEQ ID NOS: 99 and 101.

Alternatively, said pair of probes and/or primers is used to detect full-length [FL] human MN/CA9 mRNA but not alternatively-spliced [AS] human MN/CA9 mRNA. An exemplary and preferred pair of probes and/or primers used to detect FL mRNA only consists of SEQ ID NOS: 99 and 100, or nucleic acid sequences that are at least 80% homologous, more preferably at least 90% homologous, to SEQ ID NOS: 99 and 100. In a still further embodiment of the invention, the pair of probes and/or primers is used to detect both AS human MN/CA9 mRNA and FL human MN/CA9 mRNA, and said AS mRNA and said FL mRNA are differentiated by length. Preferably, said pair of probes and/or primers used to detect both AS and FL human MN/CA9 mRNA consists of SEQ ID NOS: 97 and 98, or nucleic acid sequences that are at least 80% homologous, more preferably at least 90% homologous, to SEQ ID NOS: 97 and 98.

The invention is still further directed to an isolated nucleic acid encoding an alternatively-spliced [AS] MN/CA IX in a mammal. Preferably, said AS MN/CA IX has a molecular weight of from about 43 to about 48 kilodaltons. The invention further relates to a vector that expresses such a nucleic acid or fragments thereof, a host cell comprising such a vector and/or to production of AS MN/CA IX proteins and polypeptides by recombinant, synthetic or other biological means.

An exemplary and preferred AS form of MN/CA IX encoded by said isolated nucleic acid is further characterized in that it is specifically bound by an antibody specific for the PG domain of MN/CA IX but is not bound by an antibody specific for the CA domain of MN/CA IX. In a still more preferred embodiment of the invention, said AS form of MN/CA IX is specifically bound by the M75 monoclonal antibody that is secreted from the hybridoma VU-M75, which was deposited at the American Type Culture Collection under ATCC No. HB 11128, but is not bound by the V/10 monoclonal antibody which is produced by the hybridoma VU-V/10, deposited at BCCM™/LMBP in Ghent, Belgium under Accession No. LMBP 6009CB.

More preferably, said mammal is a human, and said isolated nucleic acid is characterized in that nucleotides corresponding to exon 8 and exon 9 of MN/CA9 are deleted. Still more preferably, said isolated human nucleic acid has the nucleic acid sequence of SEQ ID NO: 108, or an isolated nucleic acid at least 80% homologous to SEQ ID NO: 108, more preferably at least 90% homologous to SEQ ID NO: 108. Preferably, the exemplary AS form of human MN/CA IX encoded by SEQ ID NO: 108 or closely related sequences, is specifically bound by the M75 monoclonal antibody that is secreted from the hybridoma VU-M75, which was deposited at the American Type Culture Collection under ATCC NO. HB 11128, but is not bound by the V/10 monoclonal antibody which is produced by the hybridoma VU-V/10, deposited at BCCM™/LMBP in Ghent, Belgium under Accession No. LMBP 6009CB.

Still further, the invention concerns antibodies or antigen-binding antibody fragments that bind specifically to AS MN/CA IX, but not to other forms of MN/CA IX. For example, such AS-specific antibodies may be an antibody or antigen-binding antibody fragment that binds specifically to the AS form of MN/CA IX, but does not bind specifically to the FL form of MN/CA IX; or an antibody or antigen binding antibody fragment that binds specifically to AS MN/CA IX, but does not bind specifically to soluble MN/CA IX (s-CA IX).

Further disclosed herein are therapeutic methods for treating preneoplastic/neoplastic disease in a mammal, wherein said disease is associated with abnormal expression of MN/CA IX, which methods comprise administering to said mammal a therapeutically effective amount of a composition comprising an agent that increases levels of alternatively-spliced [AS] MN/CA IX relative to levels of full-length [FL] MN/CA IX. Said AS MN/CA IX would also comprise any protein or polypeptide fragment of AS MN/CA IX that interferes with the activity of FL MN/CA IX. Preferably, said increased relative levels of AS MN/CA IX interfere with carbonic anhydrase activity of said FL MN/CA IX. Said agent may preferably be AS MN/CA IX itself in a physiologically acceptable carrier, a vector expressing AS MN/CA9 mRNA, an antisense oligonucleotide that blocks expression of FL MN/CA IX but not that of AS MN/CA IX, a vector expressing said antisense oligonucleotide, a FL MN/CA9 isoform-specific siRNA, or a vector expressing said FL MN/CA9 isoform-specific siRNA.

For example, said agent may be a FL MN/CA9 isoform-specific siRNA targeted to the splice junction of exons 7 and 8, exons 8 and 9, or exons 9 and 10 of MN/CA9 mRNA. Alternatively, said agent is an antisense oligonucleotide that modulates AS and/or FL MN/CA9 pre-mRNA splicing.

In another aspect, the invention concerns an oligonucleotide that increases levels of alternatively-spliced [AS] MN/CA IX relative to levels of full-length [FL] MN/CA IX, wherein said oligonucleotide is used in treatment of a preneoplastic/neoplastic disease associated with abnormal MN/CA IX expression. For example, said oligonucleotide can be an antisense oligonucleotide that is complementary to FL MN/CA9 pre-mRNA but not to AS MN/CA9 pre-mRNA; preferably, said oligonucleotide is complementary to the splice junction of exons 7 and 8, exons 8 and 9, or exons 9 and 10 of FL MN/CA9 mRNA. Alternatively, said oligonucleotide that increases levels of AS MN/CA IX relative to levels of FL MN/CA IX can be an siRNA complementary to FL MN/CA9 mRNA but not to AS MN/CA9 mRNA.

This invention also concerns an in vitro method of identifying agents capable of modulating levels of alternatively-spliced [AS] MN/CA IX, comprising contacting cells expressing AS MN/CA IX with an agent suspected of modulating the level of said AS MN/CA IX in the cells, and detecting and quantitating changes in levels of said AS MN/CA IX.

### REFERENCES

The following references are cited herein or provide updated information concerning the MN/CA9 gene and the MN/CA IX protein, or alternatively-spliced mRNAs. All the listed references as well as other references cited herein are specifically incorporated by reference.

Anderson et al., Oncogene 25: 61-69 (2006)

Alvarez-Pérez et al., MAGMA 18: 293-301 (2005)

Bartosova et al., J. Pathol. 197: 314-321 (2002)

Beasley et al., Cancer Res. 61(13): 5262-5267 (2001)

Cecchi et al., J Med Chem 48: 483441 (2005)

Chegwidden et al., EXS 90: 343-363 (2000)

Chrastina et al., Int. J Cancer 105: 873-881 (2003)

Dorai et al., Eur. J Cancer 41: 2935-2947 (2005).

Garcia-Blanco, Curr Opin Mol Ther., 7(5): 476-82 (2005)

Giatromanolaki et al., Cancer Res. 61(21): 7992-7998 (2001)

Gothie et al., J Biol Chem 275: 6922-6927 (2000)

Gut et al., Gastroenterology 123: 1889-1903 (2002)

Harris A L., Nature Rev Cancer 2: 3847 (2002)

He et al., Oncogene 25: 192-2202 (2006)

Hockel and Vaupel, Semin. Oncol. 28(2 Suppl 8): 3641 (2001)

Ivanov et al., J. Pathol. 158(3): 905-919 (2001)

Kaluz et al., Cancer Res 62: 44694477 (2002)

Kivela et al., World J Gastroenterol 11: 155-163 (2005)

Koukourakis et al., Clin. Cancer Res. 7(11): 3399-3403 (2001)

Liao et al., Am. J. Pathol. 145(3): 598-609 (1994)

Liao et al., Cancer Res. 57: 2827-2831 (1997)

Lieskovska et al., Neoplasma 46: 17-24 (1999)

Matlin et al., Nat Rev Mol Cell Biol 6: 386-398 (2005)

Morgan et al., Am J Physiol—Cell Physiol 293(2): C738-C748 (2007)

Olive et al., Cancer Res 61: 8924-8929 (2001)

Opavsky et al., Genomics 33(3): 480487 (1996)

Ortova-Gut, Gastroenterology 123: 1889-1903 (2002)

Pajares et al., Lancet Oncol., 8(4):349-57 (2007)

Pastorek et al., Oncogene 9: 2877-2888 (1994)

Pastorekova and Pastorek, Chapter 9, Carbonic Anhydrase: Its Inhibitors and Activators [eds. Supuran et al.; CRC Press (London et al.) 2004]

Pastorekova et al., Virology 187: 620-626 (1992)

Pastorekova et al., Gastroenterology 112: 398408 (1997)

Pastorekova et al., J Enz Inhib Med Chem 19: 199-229 (2004)

Potter and Harris, Br J Cancer 89: 2 -7 (2003)

Robertson et al., Cancer Res 64: 6160-6165 (2004)

Robinson and Stringer, J Cell Sci 114: 853-865 (2001)

Romero-Ramirez et al., Cancer Res 64: 5943-5947 (2004)

Roy et al., Nucl Acid Res 33: 5026-5033 (2005)

Saarnio et al., Am J Pathol 153: 279-285 (1998)

Skotheim and Nees, Int J Biochem Cell Biol., 39(7-8):143249 (2007)

Srebrow and Kornblihft, J Cell Sci.,119(Pt 13): 263541 (2006)

Supuran and Scozzafava, J. Enzyme Inhib. 15(6): 597-610 (2000)

Svastova et al., Exp Cell Res 290: 332-345 (2003)

Svastova et al., FEBS Letters 577: 439-445 (2004)

Swietach et al., Cancer Metastasis Rev, DOI 10.1007/s10555-007-9064-0 (2007)

Taconelli et al., Cancer Cell 6: 347-60 (2004)

Turner et al., Human Pathol. 28(6): 740-744 (1997)

Venables J P, BioEssays 28: 378-386 (2006)

Vermylen et al., Eur. Respir. J. 14: 806-811 (1999)

Wilton and Fletcher, Curr Gene Ther., 5(5):467-83 (2005)

Wingo et al., Biochemical and Biophysical Research Communications 288: 666-669 (2001)

Wong et al., Exp Mol Pathol 75: 124-130 (2003)

Wykoff et al., Cancer Res 60: 7075-7083 (2000)

Wykoff et al., Am. J. Pathol. 158(3): 1011-1019 (2001)

Xing Y. Front Biosci.,12: 403441 (2007)

Zatovicova et al., J Immunol Methods 282: 117-134 (2003)

Zavada et al., Int. J. Cancer 54: 268-274 (1993)

Zavada et al., Br. J. Cancer 82(11): 1808-1813 (2000)

### Abbreviations

The following abbreviations are used herein:
aa—amino acid
AS—alternative splicing
ATCC—American Type Culture Collection
bp—base pairs
BSA—bovine serum albumin
CA—carbonic anhydrase
CAM—cell adhesion molecule
CARP—carbonic anhydrase related protein
cm—centimeter
C-terminus—carboxyl-terminus
CTL—cytotoxic T lymphocytes
° C.—degrees centigrade
DEAE—diethylaminoethyl
DMEM—Dulbecco modified Eagle medium
ds—double-stranded
EDTA—ethylenediaminetetraacetate
EGFR—epidermal growth factor receptor
EIA—enzyme immunoassay
ELISA—enzyme-linked immunosorbent assay
ER—estrogen receptor
FCS—fetal calf serum
FITC—fluorescein isothiocyanate
FITC-CAI—fluorescent CA inhibitor (homosulfanilamide conjugated with FITC)
FL—full-length
FTP—DNase 1 footprinting analysis
GST—glutathione S-transferase
GST-MN—fusion protein MN glutathione-S transferase
h—hour(s)
H—hypoxia
HBS—HIF-binding site
HIF—hypoxia-inducible factor
HRE—hypoxia response element
IC—intracytoplasmic or intracellular
IF—immunofluorescence
IHC—immunohistochemistry
IL-2—interleukin-2
IP—immunoprecipitation with the Protein A Sepharose kb—kilobase
kd or kDa—kilodaltons
KS—keratan sulphate
M—molar
Mab or mab—monoclonal antibody
min.—minute(s)
mg—milligram
ml—milliliter
mM—millimolar
M-MuLV—murine leukemia virus
N—normal concentration; normoxia
ND—notdone
ng—nanogram
nt—nucleotide
N-terminus—amino-terminus
ODN—oligodeoxynucleotide
ORF—open reading frame
PAGE—polylacrylamide gel electrophoresis
PBS—phosphate buffered saline
PCR—polymerase chain reaction
PG—proteoglycan-like region
pl—isoelectric point
RACE—rapid amplification of CDNA ends
RCC—renal cell carcinoma
RIA—radioimmunoassay
RIPA—radioimmunoprecipitation assay
RNP—RNase protection assay
RT-PCR—reverse transcriptase polymerase chain reaction
SDS—sodium dodecyl sulfate
SDS-PAGE—sodium dodecyl sulfate-polyacrylamide gel electrophoresis
SP—signal peptide
TC—tissue culture
tk—thymidine kinase
TM—transmembrane
Tris—tris (hydroxymethyl) aminomethane
μg—microgram
μl—microliter
μM—micromolar
VEGF—vascular endothelial growth factor

### Cell Lines

ACHN—human kidney carcinoma
C33a—human cervical carcinoma cells [ATCC HTB-31; J. Natl. Cancer Inst. (Bethesda) 32: 135 (1964)]
CAKI-1—human kidney carcinoma
Caski—human cervical carcinoma
CGL1—H/F-N hybrid cells (HeLa D98/AH.2 derivative)
CGL2—H/F-N hybrid cells (HeLa D98/AH.2 derivative)
CGL3—H/F-T hybrid cells (HeLa D98/AH.2 derivative)
CGL4—H/F-T hybrid cells (HeLa D98/Ah.2 derivative)
HeLa—human cervical carcinoma; from American Type Culture Collection (ATCC)
MDCK—canine epithelial cell line, derived from a kidney from an apparently normal adult female cocker spaniel by S. H. Madin and N. B. Barby in 1958. (ATCC CCL-34)
NIH3T3—murine fibroblast cell line reported in Aaronson, Science, 237: 178 (1987)
SiHa—human cervical squamous carcinoma cell line [ATCC HTB-35; Friedl et al., Proc. Soc. Exp. Biol. Med., 135: 543 (1990)]

### Nucleotide and Amino Acid Symbols

The following symbols are used to represent nucleotides herein:

| Base | |
|---|---|
| Symbol | Meaning |
| A | adenine |
| C | cytosine |
| G | guanine |
| T | thymine |
| U | uracil |
| I | inosine |
| M | A or C |
| R | A or G |
| W | A or T/U |
| S | C or G |
| Y | C or T/U |
| K | G or T/U |
| V | A or C or G |
| H | A or C or T/U |
| D | A or G or T/U |
| B | C or G or T/U |
| N/X | A or C or G or T/U |

There are twenty main amino acids, each of which is specified by a different arrangement of three adjacent nucleotides (triplet code or codon), and which are linked together in a specific order to form a characteristic protein. A three-letter or one-letter convention is used herein to identify said amino acids, as, for example, in FIG. 1 as follows:

| | 3 Ltr. | 1 Ltr. |
|---|---|---|
| Amino acid name | Abbrev. | Abbrev. |
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Unknown or other | | X |

### BRIEF DESCRIPTION OF THE FIGURES

FIG. 1 provides identification and predicted structure of the mouse splicing variant of CA IX. (A) Genomic structure of the mouse Car9 gene (GenBank # AY049077). (B) RT-PCR of Car9 splicing variants in the mouse gastrointestinal tissues. [See Table 2, infra, for the sequences of primers used in the Examples.] (C) Separate amplification of the FL and AS transcripts. (D) Comparison of the FL and AS amino acid sequences. (E) Predicted structure of the mouse AS CA IX protein.

FIG. 2 depicts immunoblotting analysis and localization of the mouse AS CA IX. pSG5C-AS plasmid containing the mouse AS cDNA was transfected to NIH3T3 and MDCK cells, respectively. (A) Immunoblotting analysis of AS-transfected cells using the polyclonal serum against the mouse CA IX shows a single AS-related band. (B) Immunofluorescence analysis of the transfectants demonstrates an intracellular localization of the mouse AS protein.

FIG. 3 provides identification and predicted structure of the human splicing variant of CA IX. (A) Schematic illustration of the genomic structure of the human CA9 gene (GenBank # Z54349). Positions of primers are indicated by arrows. [See Table 2 for the sequences of primers used in the Examples.] Exons excluded by alternative splicing are in dark grey color. (B) RT-PCR analysis of CA9 in the human stomach and intestine using h1S-h6A primers [SEQ ID NOS: 95 and 96] that do not discriminate between the splicing variants. (C) Amplification of both FL and AS transcripts in the human tissues using h6S-h11A primers [SEQ ID NOS: 97 and 98]. (D) Comparison of amino-acid sequences deduced from the human FL and AS CA9 cDNAs. Signal peptide (SP) is written in italic, proteoglycan-like domain (PG) is in bold, carbonic anhydrase domain (CA) is boxed by solid lines, the transmembrane region (TM) which starts at amino acid (aa) 415, is boxed by broken lines. Dashed lines represent amino acid residues deleted in AS. Histidines that bind a catalytic zinc and cysteines involved in formation of S-S bonds are singly boxed by broken lines. (E) Predicted structure of the human FL and AS CA IX proteins.

FIG. 4 shows the expression of the AS CA IX variant in human tumor cell lines and in human tissues, with RT-PCR analysis of human AS CA9 using the primers designed for individual amplification of the splicing variants, namely h7S-h8A [SEQ ID NOS: 99 and 100] for FL and h7S-h10/7A [SEQ ID NOS: 99 and 101] for AS (see FIG. 3). Beta-actin was used as a standard. The cDNAs were isolated (A) from the cells exposed to normoxia (N) and hypoxia (H) for 48 h, (B) from the cells incubated at low and high density for 72 h, and (C) from normal and tumor human tissues. The results indicate that the AS expression is steady and does not depend on hypQxia, density and tumor phenotype.

FIG. 5 depicts localization and oligomerization of the human AS CA IX. CA IX-negative MDCK cells and HeLa cells with natural hypoxia-induced expression of FL CA IX were permanently transfected with AS CA9 cDNA in pSG5C plasmid. (A) Immunofluorescence analysis of the AS-transfected (AS), FL-transfected (FL) and control cells (mock) was performed using M75 MAb recognizing both AS and FL proteins. (B) Immunoblotting analysis of the protein extracts and media from HeLa-AS and control HeLa cells. The AS CA IX variant was detected with M75 MAb in extract as well as in medium of AS-transfected cells.

FIG. 6 shows the ability of FL and AS splicing variants to form oligomers. (A) Non-reducing SDS-PAGE and immunoblotting with M75 showed that AS is unable to form oligomers. (B) Detection of splicing variants in oligomers by immunoprecipitation from HeLa-AS extract with MAb V/10 (recognizes FL but not AS) or M75 (recognizes both variants). Components of the precipitated oligomers were visualized using peroxidase-labelled M75.

FIG. 7 shows the effect of overexpressed AS variant on acidification, inhibitor binding and spheroid formation. (A) The AS-transfected HeLa cells and related mock-transfected controls were incubated for 48 h in normoxia and hypoxia, respectively, and extracellular pH was measured in culture medium immediately at the end of experiment. Data are expressed as differences between the pH values (ΔpH) measured in normoxic versus hypoxic cells and include standard deviations. Results show that expression of AS reduces the acidification mediated by FL CA IX protein under hypoxia. (B) MDCK-CA IX transfected cells that constitutively express human FL CA IX protein were treated for 48 h by a fluorescent CA inhibitor (FITC-CAI) in the absence (control) or in the presence of the secreted AS variant added with the conditioned medium from MDCK-AS transfectants. Conditioned medium was mixed with a fresh cultivation medium. FITC-CAI bound only to hypoxic cells and was considerably reduced in the presence of the AS protein. (C) The same experiment was performed repeatedly with either one half (½ AS) or one third (⅓ AS) of conditioned medium from MDCK-AS cells. Binding of FITC-CAI and corresponding fluorescence was evaluated from acquired images using Scion Image software. Data were expressed as a percentage of positive control represented by hypoxic MDCK-CA IX cells incubated with FITC-CAI in the absence of AS. The results confirmed that AS reduces the binding of FITC-CAI to CA IX. (D) Microscopic images of spheroids grown from control mock-transfected HeLa cells and from AS-transfected HeLa cells, respectively. Control HeLa cells express hypoxia-induced, functional FL CA IX protein and produce spheroids that form compact cores. HeLa-AS cells, which contain both hypoxia-induced FL CA IX and constitutively expressed AS, contain loose cores possibly due to AS-compromised function of FL leading to decreased survival of hypoxic core cells.

FIG. 8A-C provides the nucleotide sequence for a MN cDNA [SEQ ID NO: 1] clone isolated as described herein. FIG. 8A-C also sets forth the predicted amino acid sequence [SEQ ID NO: 2] encoded by the cDNA.

FIG. 9A-F provides a 10,898 bp complete genomic sequence of MN [SEQ ID NO: 3]. The base count is as follows: 2654 A; 2739 C; 2645 G; and 2859 T. The 11 exons are in general shown in capital letters, but exon 1 is considered to begin at position 3507 as determined by RNase protection assay.

FIG. 10 is a nucleotide sequence for the proposed promoter of the human MN gene [SEQ ID NO: 24]. The nucleotides are numbered from the transcription initiation site according to RNase protection assay. Potential regulatory elements are overlined. Transcription start sites are indicated by asterisks (RNase protection) and dots (RACE) above the corresponding nucleotides. The sequence of the 1 st exon begins under the asterisks. FTP analysis of the MN4 promoter fragment revealed 5 regions (I-V) protected at both the coding and noncoding strands, and two regions (VI and VII) protected at the coding strand but not at the noncoding strand.

### DETAILED DESCRIPTION

The MN/CA IX protein is functionally implicated in tumorigenesis as part of the regulatory mechanisms that control pH and cell adhesion. MN/CA IX is induced primarily under hypoxia via the HIF-1 pathway; HIF-1 may also be expressed under normoxia by different extracellular signals and oncogenic changes, such as high cell density, transmitted via the PI3K pathway, which can result in increased MN/CA IX expression. Both the HIF-1 and PI3K pathways increase HIF-1 protein levels, which increases can be translated into increased MN/CA IX levels.

The inventors found, as shown in the Examples below, that in addition to full-length (FL) CA9 transcript encoding a hypoxia-induced CA IX protein with high enzyme activity and capacity to regulate pH, there is also a less abundant, constitutively-produced, alternatively-spliced (AS) CA9 transcript. As demonstrated in Example 2 below, the alternative splicing variant of the human CA9 mRNA does not contain exons 8 and 9 and is expressed in tumor cells independently of hypoxia. It is also detectable in normal tissues in the absence of the full-length transcript and can therefore produce false-positive data in prognostic studies based on detection of the hypoxia- and cancer-related CA9 expression. The splicing variant encodes a truncated CA IX protein lacking the C-terminal part of the catalytic domain, shows diminished catalytic activity, and is either localized intracellularly or secreted. When overexpressed, it reduces the capacity of the full-length CA IX protein to acidify extracellular pH of hypoxic cells and to bind carbonic anhydrase inhibitor. Examples 4 and 5 describe experiments showing that the human AS CA IX variant is not confined to plasma membrane and upon overexpression interferes with the function of the FL protein. In Example 5, HeLa cells transfected with the splicing variant cDNA generate spheroids that do not form compact cores, suggesting that they fail to adapt to hypoxic stress. This AS capability may be relevant particularly under conditions of mild hypoxia, when the cells do not suffer from severe acidosis and do not need excessive pH control.

The hypoxia- and tumor- independent production of the AS variant of CA IX has many implications for diagnostic, prognostic and therapeutic aspects of CA IX. Future diagnostic/prognostic studies of the full-length CA9 mRNA (encoding the functional CA IX protein) can design probes/primers designed to avoid simultaneous detection of an alternatively spliced variant, and cancer therapies can be based on CA9 alternative splicing, e.g., by design of oligonucleotides used for antisense and RNA interference therapies, among other therapies.

### Preneoplastic/Neoplastic Diseases

The preneoplastic/neoplastic diseases (and affected tissues) that are the subject of the diagnostic/prognostic and therapeutic methods of the invention are those that are associated with abnormal expression of MN/CA IX. As used herein, “preneoplastic/neoplastic tissues” may also include preneoplastic/neoplastic cells within body fluids. Preferably, said preneoplastic/neoplastic disease is selected from the group consisting of mammary, urinary tract, bladder, kidney, ovarian, uterine, cervical, endometrial, squamous cell, adenosquamous cell, vaginal, vulval, prostate, liver, lung, skin, thyroid, pancreatic, testicular, brain, head and neck, mesodermal, sarcomal, stomach, spleen, gastrointestinal, esophageal, and colon preneoplastic/neoplastic diseases.

### Normoxia and Hypoxia

As used herein, “normoxia” is defined as oxygen tension levels in a specific mammalian tissue that are within the normal ranges of physiological oxygen tension levels for that tissue. As used herein, “hypoxia” is defined as an oxygen tension level necessary to stabilize HIF-1α in a specific tissue or cell. Experimentally-induced hypoxia is generally in the range of 2% pO₂ or below, but above anoxia (0% pO₂, as anoxia would be lethal). The examples described herein that concern hypoxia were performed at 2% pO₂ which is an exemplary hypoxic condition. However, ones of skill in the art would expect other oxygen tension levels to be understood as “hypoxic” and to produce similar experimental results. For example, Wykoff et al. [Cancer Research. 60: 7075-7083 (2000)] used a condition of 0.1% pO₂ as representative of hypoxia to induce HIF-1α-dependent expression of CA9. Tomes et al. has demonstrated varying degrees of HIF-1α stabilization and CA9 expression in HeLa cells or primary human breast fibroblasts under exemplary in vitro hypoxic conditions of 0.3%, 0.5% and 2.5% pO₂ [Tomes et al., Br. Cancer Res. Treat. 81(1):61-69 (2003)]. Alternatively, Kaluz et al. has used the exemplary hypoxic condition of 0.5% pO₂ for experimental induction of CA9 [Kaluz et al., Cancer Res., 63: 917-922 (2003)] and referred to “experimentally-induced ranges” of hypoxia as 0.1-1% pO₂ [Cancer Res., 62: 44694477 (2002)].

Oxygen tension levels above 2% pO₂ may also be hypoxic, as shown by Tomes et al., supra. One of skill in the art would be able to determine whether a condition is hypoxic as defined herein, based on a determination of HIF-1α stabilization. Exemplary ranges of hypoxia in a specific tissue or cell may be, for example, between about 3% to about 0.05% pO₂, between about 2% to about 0.1% pO₂, between about 1% to about 0.1% pO₂, and between about 0.5% to about 0.1% pO₂.

### MN Gene and Protein

The terms “CA IX” and “MN/CA9” are herein considered to be synonyms for MN. Also, the G250 antigen is considered to refer to MN protein/polypeptide.

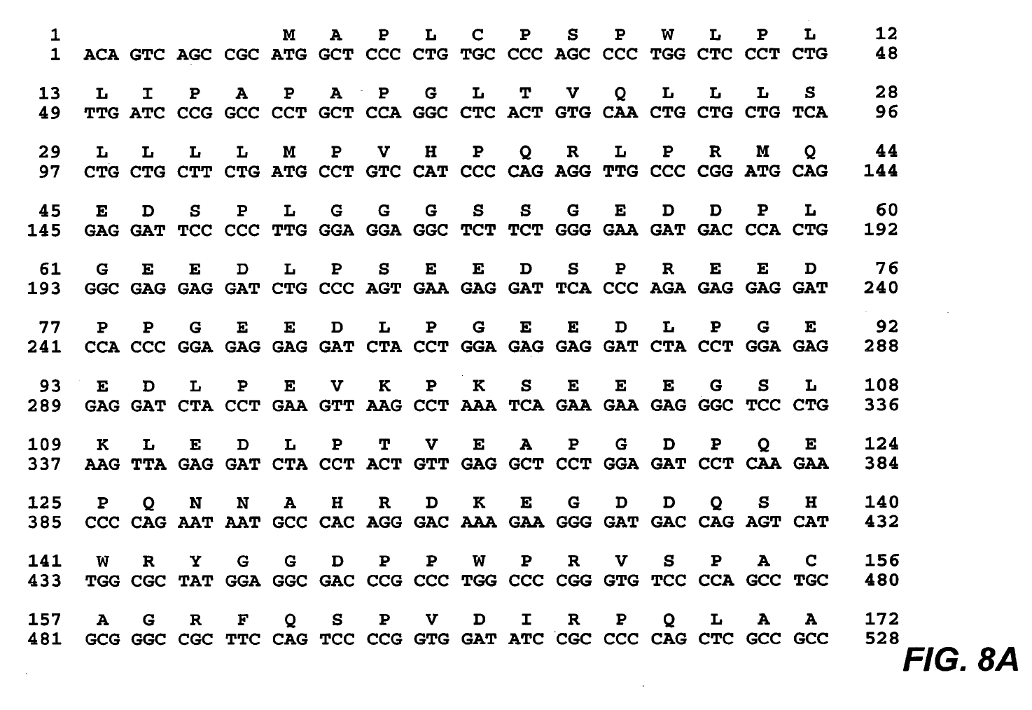

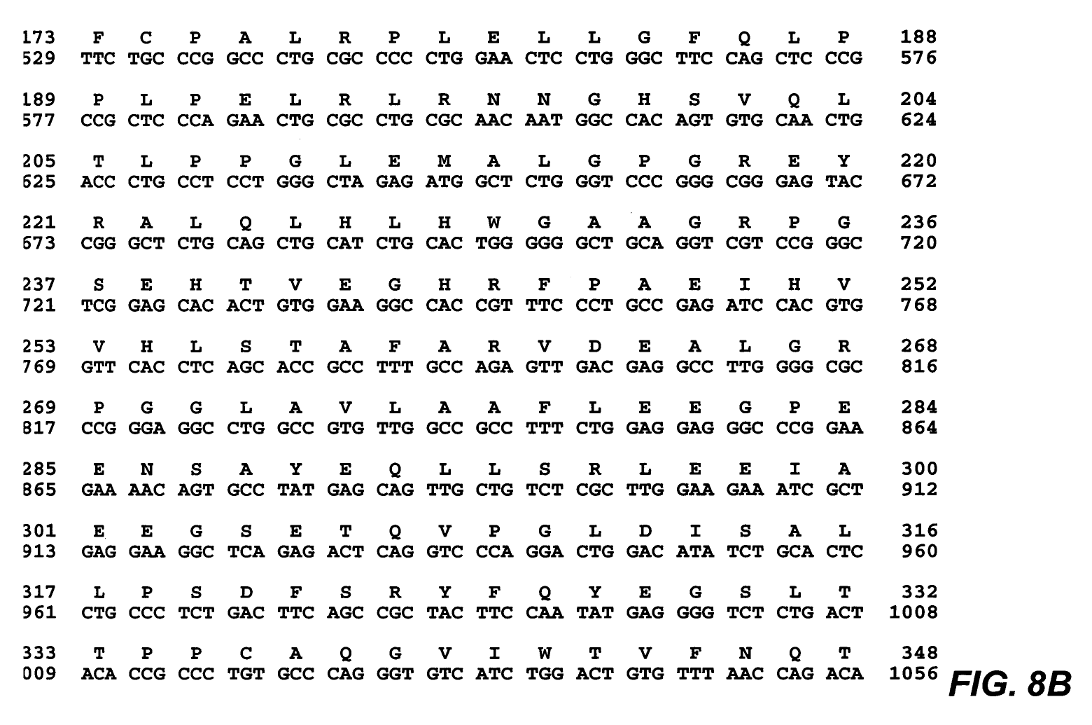

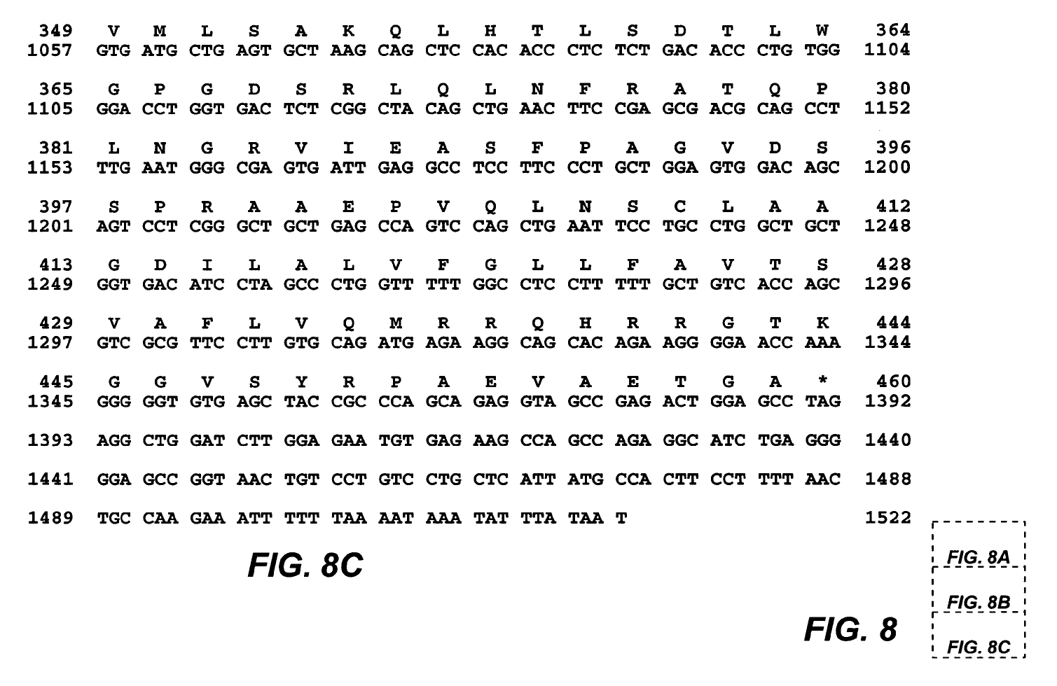

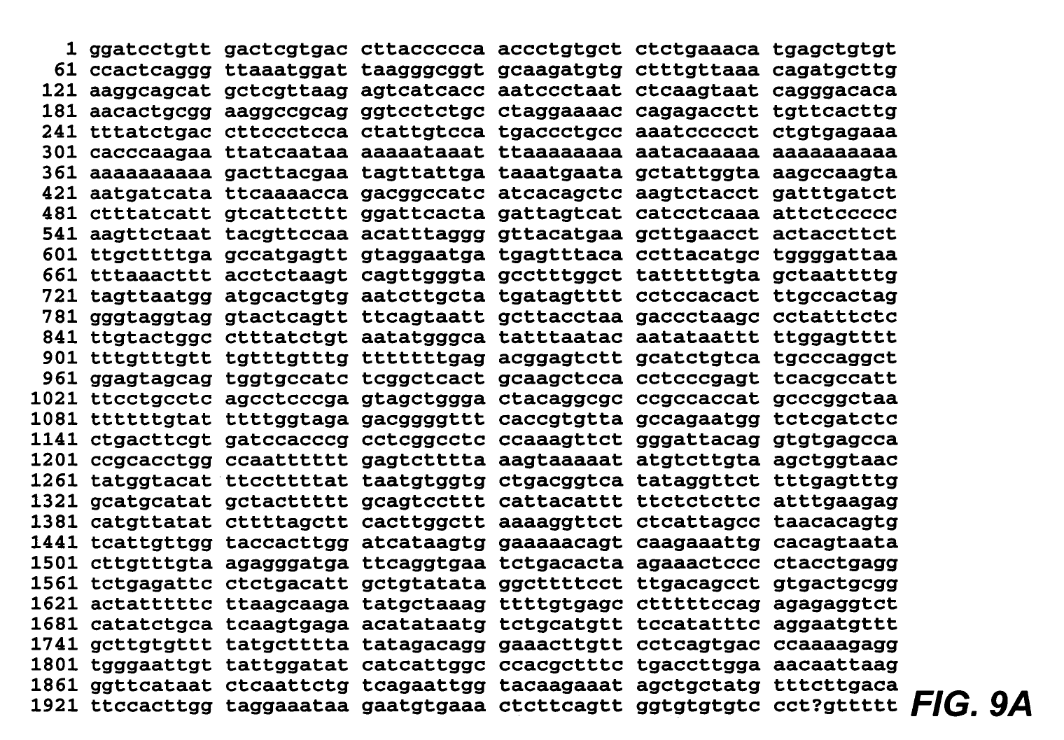

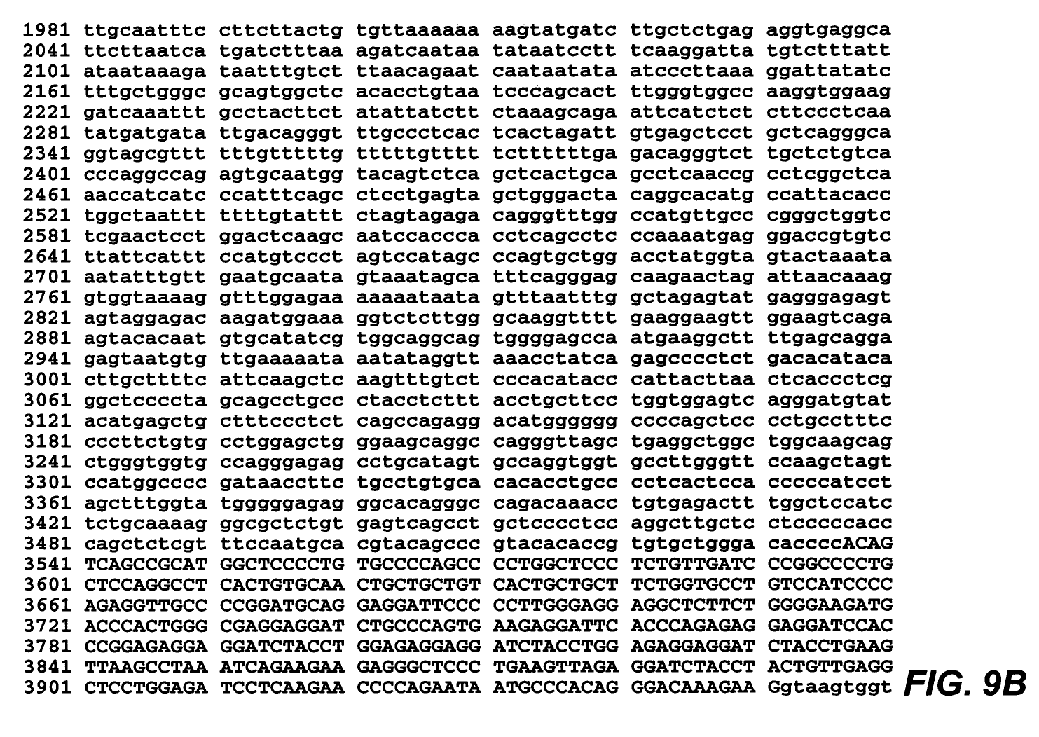

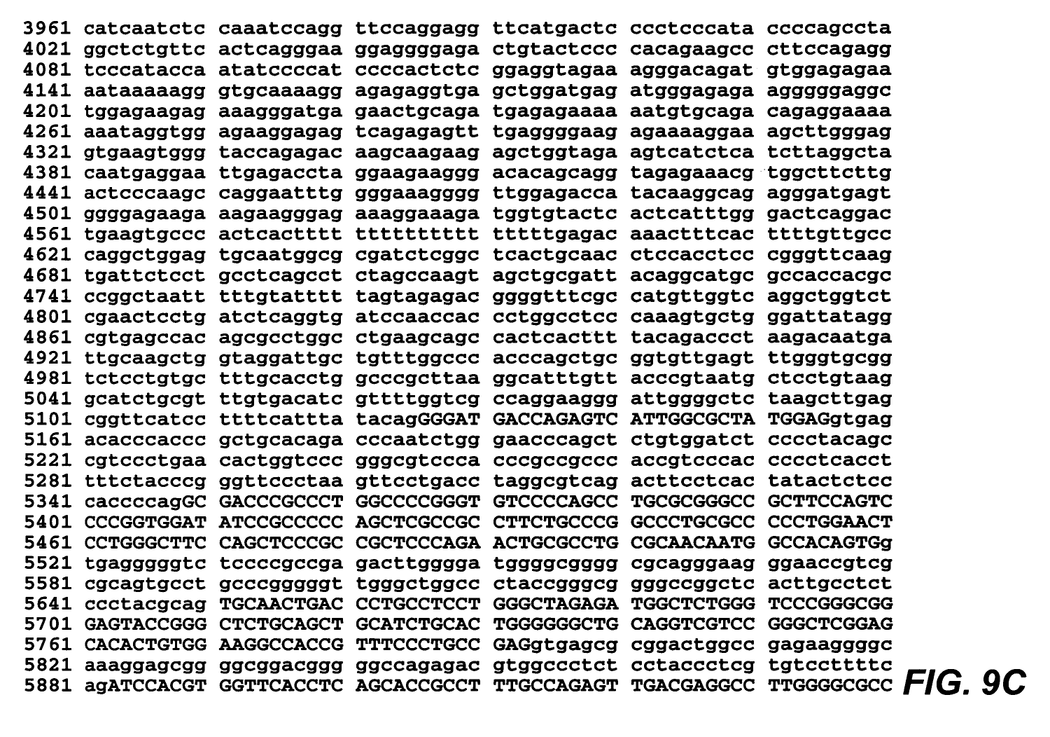

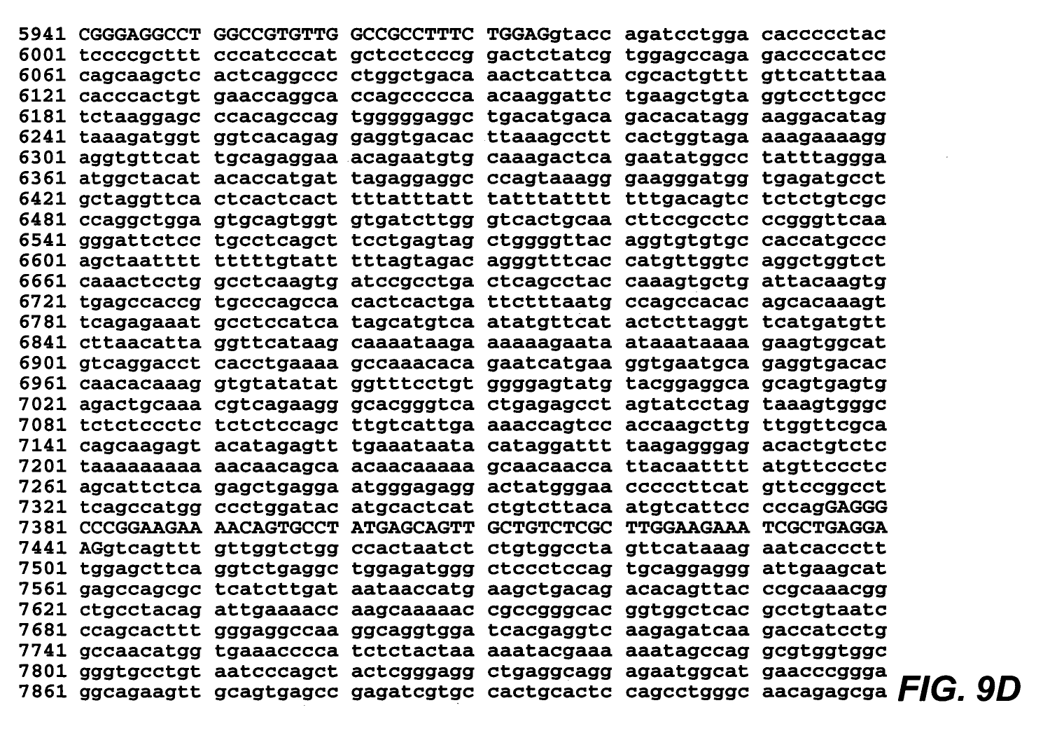

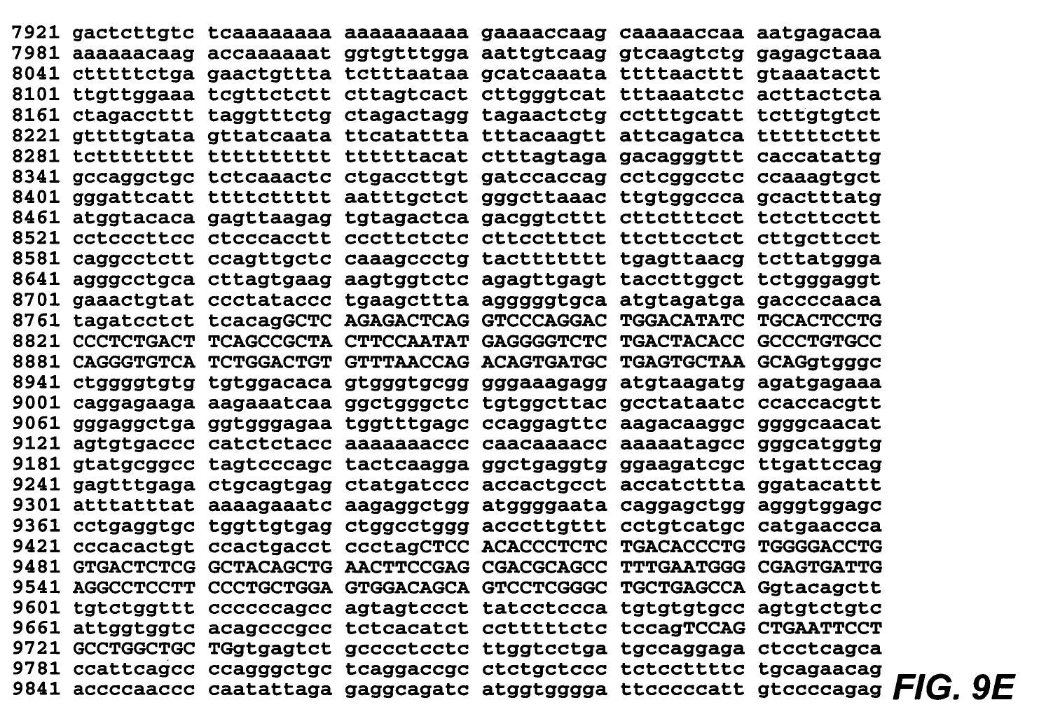

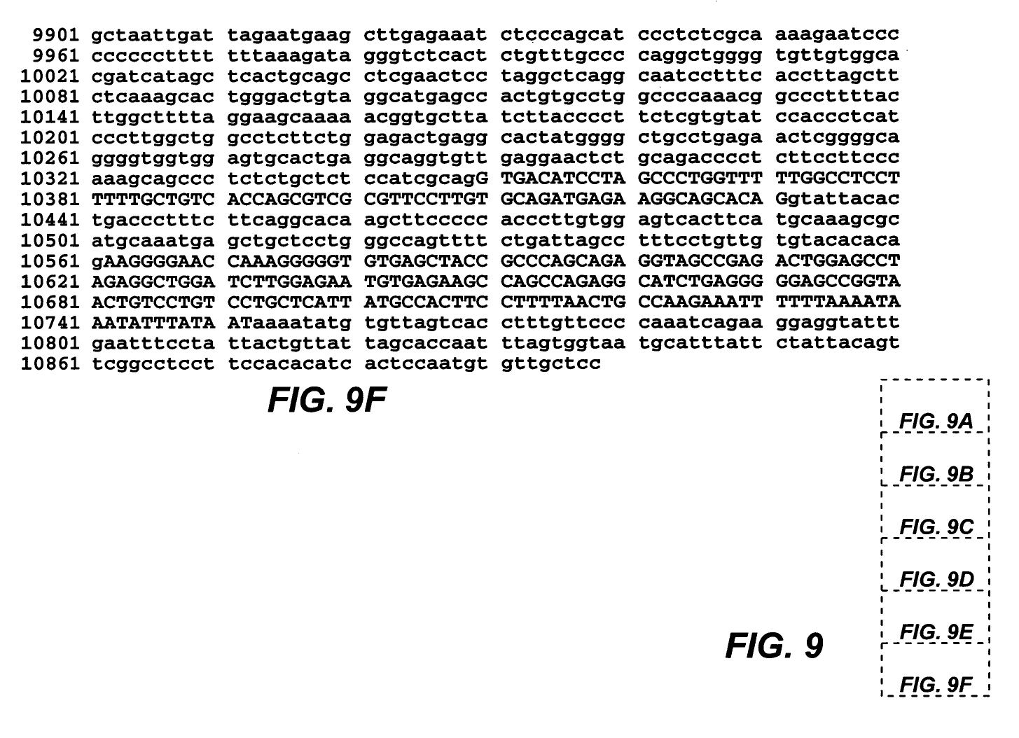

Zavada et al., WO 93/18152 and/or WO 95/34650 disclose the MN cDNA sequence shown herein in FIG. 8 [SEQ ID NO: 1], the MN amino acid sequence [SEQ ID NO: 2] also shown in FIG. 8, and the MN genomic sequence [SEQ ID NO: 3] shown herein in FIG. 9. The MN gene is organized into 11 exons and 10 introns.

The ORF of the MN cDNA shown in FIG. 8 has the coding capacity for a 459 amino acid protein with a calculated molecular weight of 49.7 kd. The overall amino acid composition of the MN protein is rather acidic, and predicted to have a pl of 4.3. Analysis of native MN protein from CGL3 cells by two-dimensional electrophoresis followed by immunoblotting has shown that in agreement with computer prediction, the MN is an acidic protein existing in several isoelectric forms with pls ranging from 4.7 to 6.3.

The first thirty seven amino acids of the MN protein shown in FIG. 8 is the putative MN signal peptide [SEQ ID NO: 4]. The MN protein has an extracellular domain [amino acids (aa) 38-414 of FIG. 8 [SEQ ID NO: 5], a transmembrane domain [aa 415-434; SEQ ID NO: 6] and an intracellular domain [aa 435-459; SEQ ID NO: 7]. The extracellular domain contains the proteoglycan-like domain [aa 53-111: SEQ ID NO: 8] and the carbonic anhydrase (CA) domain [aa 135-391; SEQ ID NO: 9].

The CA domain is essential for induction of anchorage independence, whereas the TM anchor and IC tail are dispensable for that biological effect. The MN protein is also capable of causing plasma membrane ruffling in the transfected cells and appears to participate in their attachment to the solid support. The data evince the involvement of MN in the regulation of cell proliferation, adhesion and intercellular communication.

### MN Gene—Cloning and Sequencing

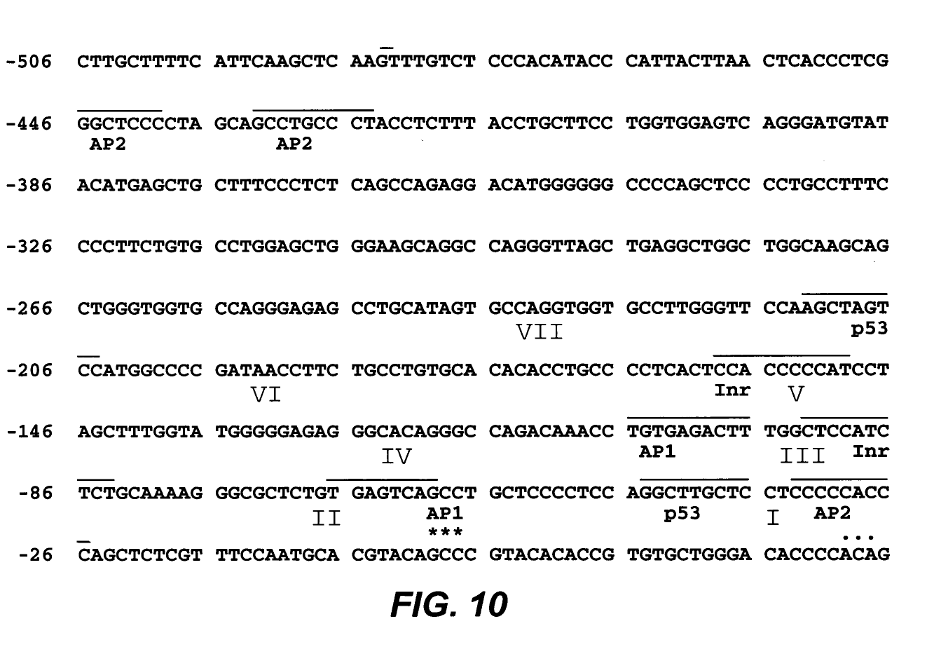

FIG. 8A-C provides the nucleotide sequence for a full-length MN cDNA clone [SEQ ID NO: 1]. FIG. 9A-F provides a complete MN genomic sequence [SEQ ID NO: 3]. The nucleotide sequence for a proposed MN promoter [SEQ ID NO: 24] is shown in FIG. 9A-F at nts 3001 to 3540, and in FIG. 10.

It is understood that because of the degeneracy of the genetic code, that is, that more than one codon will code for one amino acid [for example, the codons TTA, TTG, CTT, CTC, CTA and CTG each code for the amino acid leucine (leu)], that variations of the nucleotide sequences in, for example, SEQ ID NOS: 1 and 3 wherein one codon is substituted for another, would produce a substantially equivalent protein or polypeptide according to this invention. All such variations in the nucleotide sequences of the MN cDNA and complementary nucleic acid sequences are included within the scope of this invention.

It is further understood that the nucleotide sequences herein described and shown in FIGS. 8, 9 and 10 represent only the precise structures of the cDNA, genomic and promoter nucleotide sequences isolated and described herein. It is expected that slightly modified nucleotide sequences will be found or can be modified by techniques known in the art to code for substantially similar or homologous MN proteins and polypeptides, for example, those having similar epitopes, and such nucleotide sequences and proteins/polypeptides are considered to be equivalents for the purpose of this invention.

DNA or RNA having equivalent codons is considered within the scope of the invention, as are synthetic nucleic acid sequences that encode proteins/polypeptides homologous or substantially homologous to MN proteins/polypeptides, as well as those nucleic acid sequences that would hybridize to said exemplary sequences [SEQ. ID. NOS. 1, 3 and 24] under stringent conditions, or that, but for the degeneracy of the genetic code would hybridize to said cDNA nucleotide sequences under stringent hybridization conditions. Modifications and variations of nucleic acid sequences as indicated herein are considered to result in sequences that are substantially the same as the exemplary MN sequences and fragments thereof.

Only very closely related nt sequences having a homology of at least 80-90%, preferably at least 90%, would hybridize to each other under stringent conditions. A sequence comparison of the MN CDNA sequence shown in FIG. 8 and a corresponding cDNA of the human carbonic anhydrase II (CA II) showed that there are no stretches of identity between the two sequences that would be long enough to allow for a segment of the CA II cDNA sequence having 25 or more nucleotides to hybridize under stringent hybridization conditions to the MN cDNA or vice versa.

Stringent hybridization conditions are considered herein to conform to standard hybridization conditions understood in the art to be stringent. For example, it is generally understood that stringent conditions encompass relatively low salt and/or high temperature conditions, such as provided by 0.02 M to 0.15 M NaCl at temperatures of 50° C. to 70° C. Less stringent conditions, such as, 0.15 M to 0.9 M salt at temperatures ranging from 20° C. to 55° C. can be made more stringent by adding increasing amounts of formamide, which serves to destabilize hybrid duplexes as does increased temperature.

Exemplary stringent hybridization conditions are described in Sambrook et al., Molecular Cloning: A Laboratory Manual, pages 1.91 and 9.47-9.51 (Second Edition, Cold Spring Harbor Laboratory Press; Cold Spring Harbor, N.Y.; 1989); Maniatis et al., Molecular Cloning: A Laboratory Manual, pages 387-389 (Cold Spring Harbor Laboratory; Cold Spring Harbor, N.Y.; 1982); Tsuchiya et al., Oral Surgery, Oral Medicine, Oral Pathology, 71(6): 721-725 (June 1991); and in U.S. Pat. No. 5,989,838, U.S. Pat. No. 5,972,353, U.S. Pat. No. 5,981,711, nd U.S. Pat. No. 6,051,226.

Plasmids containing the MN genomic sequence (SEQ ID NO: 3)—the A4a clone and the XE1 and XE3 subclones—were deposited at the American Type Culture Collection (ATCC) on Jun. 6, 1995, respectively under ATCC Deposit Nos. 97199, 97200, and 97198.

### Exon-Intron Structure of Complete MN Genomic Region

The complete sequence of the overlapping clones contains 10,898 bp (SEQ ID NO: 3). The human MN gene comprises 11 exons as well as 2 upstream and 6 intronic Alu repeat elements. All the exons are small, ranging from 27 to 191 bp, with the exception of the first exon which is 445 bp. The intron sizes range from 89 to 1400 bp. The CA domain is encoded by exons 2-8, while the exons 1,10 and 11 correspond respectively to the proteoglycan-like domain, the transmembrane anchor and cytoplasmic tail of the MN/CA IX protein. Table 1 below lists the splice donor and acceptor sequences that conform to consensus splice sequences including the AG-GT motif [Mount, Nucleic Acids Res. 10: 459472 (1982)].

TABLE 1
Exon-Intron Structure of the Human MN Gene

| | | Genomic | SEQ | 5′splice | SEQ |
|---|---|---|---|---|---|
| Exon | Size | Position ** | ID NO | donor | ID NO |
| 1 | 445 | *3507-3951 | 25 | AGAAG gtaagt | 46 |
| 2 | 30 | 5126-5155 | 26 | TGGAG gtgaga | 47 |
| 3 | 171 | 5349-5519 | 27 | CAGTC gtgagg | 48 |
| 4 | 143 | 5651-5793 | 28 | CCGAG gtgagc | 49 |
| 5 | 93 | 5883-5975 | 29 | TGGAG gtacca | 50 |
| 6 | 67 | 7376-7442 | 30 | GGAAG gtcagt | 51 |
| 7 | 158 | 8777-8934 | 31 | AGCAG gtgggc | 52 |
| 8 | 145 | 9447-9591 | 32 | GCCAG gtacag | 53 |
| 9 | 27 | 9706-9732 | 33 | TGCTG gtgagt | 54 |
| 10 | 82 | 10350-10431 | 34 | CACAG gtatta | 55 |
| 11 | 191 | 10562-10752 | 35 | ATAAT end | |
| | | Genomic | SEQ | 3′splice | SEQ |
| Intron | Size | Position ** | ID NO | acceptor | ID NO |
| 1 | 1174 | 3952-5125 | 36 | atacag GGGAT | 56 |
| 2 | 193 | 5156-5348 | 37 | ccccag GCGAC | 57 |
| 3 | 131 | 5520-5650 | 38 | acgcag TGCAA | 58 |
| 4 | 89 | 5794-5882 | 39 | tttcag ATCCA | 59 |
| 5 | 1400 | 5976-7375 | 40 | ccccag GAGGG | 60 |
| 6 | 1334 | 7443-8776 | 41 | tcacag GCTCA | 61 |
| 7 | 512 | 8935-9446 | 42 | ccctag CTCCA | 62 |
| 8 | 114 | 9592-9705 | 43 | ctccag TCCAG | 63 |
| 9 | 617 | 9733-10349 | 44 | tcgcag GTGACA | 64 |
| 10 | 130 | 10432-10561 | 45 | acacag AAGGG | 65 |

** positions are related to nt numbering in whole genomic sequence including the 5′ flanking region [FIGS. 9A-F]
*number corresponds to transcription initiation site determined below by RNase protection assay

### Mapping of MN Gene Transcription Initiation and Termination Sites

Zavada et al., WO 95/34650 describes the process of mapping the MN gene transcription initiation and termination sites. A RNase protection assay was used for fine mapping of the 5′ end of the MN gene. The probe was a uniformly labeled 470 nucleotide copy RNA (nt −205 to +265) [SEQ ID NO: 66], which was hybridized to total RNA from MN-expressing HeLa and CGL3 cells and analyzed on a sequencing gel. That analysis has shown that the MN gene transcription initiates at multiple sites, the 5′ end of the longest MN transcript being 30 nt longer than that previously characterized by RACE.

Mouse Car9 cDNA

Cloning and characterization of the cDNA and gene encoding the mouse CA IX has been described previously [Ortova-Gut et al., Gastroenterology, 123: 1889-1903 (2002)]. Mouse Car9 cDNA fragment was isolated by RT PCR using primers derived from the human cDNA and the template RNA isolated from the stomach of C57 BU6J mouse. The full-length cDNA was obtained by rapid amplification of cDNA ends in both 5′/3′ directions. It encompasses 1982 bp composed of 49 bp 5′ untranslated region, 1311 bp open reading frame and 622 bp 3′ untranslated sequence (deposited in EMBL database under the Accession No. AJ245857; SEQ ID NO: 71).

The Car9 cDNA has a coding capacity for a 437 amino acid protein (deposited in EMBL database under the Accession No. CAC80975 (Q8VDE4); SEQ ID NO: 73) with a theoretical molecular mass of 47.3 kDa. The mouse protein shows 69.5% sequence identity with its human homologue and has a similar predicted domain arrangement [Opavsky et al. (1996)]. Amino acids (aa) 1-31 (SEQ ID NO: 74) correspond to a signal peptide. The N-terminal extracellular region of the mature protein (aa 32-389) (SEQ ID NO: 75) is composed of a proteoglycan-like region (aa 48-107) (SEQ ID NO: 76), and a carbonic anhydrase domain (aa 112-369) (SEQ ID NO: 77). The C-terminal region (aa 390437) (SEQ ID NO: 78) consists of the transmembrane anchor (aa 390411) (SEQ ID NO: 79) and a short cytoplasmic tail (aa 412437) (SEQ ID NO: 80). Most of the sequence differences between the mouse and human CA IX were found within the proteoglycan-like (PG) region, while the CA domain revealed the highest conservation. However, out of the five key amino acids involved in the enzyme active site (His⁹⁴, His⁹⁰, Glu¹⁰⁶, His¹¹⁹, Thr¹⁹⁹) [Christianson and Cox, Annu. Rev. Biochem., 68: 33-57 (1999)] all are preserved in human CA IX, but one is altered in the mouse isoenzyme (Thr¹⁹⁹→Ser). Despite that substitution, the mouse CA IX bound to a sulfonamide agarose suggesting that it may possess an enzyme activity.

Availability of Car9 cDNA allowed the analysis of the expression pattern of Car9 mRNA in mouse tissues. A ribonuclease protection assay (RNP) was carried out with a riboprobe of 170 bp designed to detect the 3′ part of the region encoding the CA domain. As expected on the basis of the distribution in human and rat tissues, the highest level of Car9 mRNA was detected in the mouse stomach. Medium level of Car9 mRNA was found in the small intestine and colon, while the kidney and brain showed very weak expression. The liver and spleen were negative. Noteworthy, the RNP signal was also present in the mouse embryo at the age of embryonic day E18.5, but not in embryonic stem cells and in the E10.5 embryo. That may suggest a role for CA IX in the development of the mouse gastrointestinal tract.

Organization of Mouse Car9 Gene

In order to isolate the Car9 gene and determine its organization, the full-length Car9 cDNA was used for screening of a mouse embryonic stem cell 129/Ola genomic library in pBAC108L. Obtained was one BACM-355(G13) clone that contained complete Car9 genomic sequence as confirmed by restriction mapping and Southern blot analysis of a mouse wild type genomic DNA. Three overlapping genomic fragments derived from this clone were subcloned into pBluescript II KS.

Analysis of the genomic sequence (GenBank Accession No. AY049077; SEQ ID NO: 72) revealed that the Car9 gene covers 6.7 kb of the mouse genome and consists of 11 exons and 10 introns. Distribution of introns and exon-to-protein domain relationships are similar to the human counterpart [Opavsky et al. (1996)]. The Southern hybridization pattern indicated that Car9 is a single copy gene. The EcoRI-HindIII fragment encompassing 5.9 kb and spanning the promoter region and exons 1-6 was used for a construction of the targeting vector.

### MN Proteins and/or Polypeptides

The phrase “MN proteins and/or polypeptides” (MN proteins/polypeptides) is herein defined to mean proteins and/or polypeptides encoded by an MN gene or fragments thereof. An exemplary and preferred MN protein according to this invention has the deduced amino acid sequence shown in FIG. 8. Preferred MN proteins/polypeptides are those proteins and/or polypeptides that have substantial homology with the MN protein shown in FIG. 8. For example, such substantially homologous MN proteins/polypeptides are those that are reactive with the MN-specific antibodies of this invention, preferably the Mabs M75, V/10, MN12, MN9 and MN7 or their equivalents. The VU-M75 hybridoma that secretes the M75 Mab was deposited at the ATCC under HB 11128 on Sep. 17, 1992.

A “polypeptide” or “peptide” is a chain of amino acids covalently bound by peptide linkages and is herein considered to be composed of 50 or less amino acids. A “protein” is herein defined to be a polypeptide composed of more than 50 amino acids. The term polypeptide encompasses the terms peptide and oligopeptide. As used herein, “AS MN/CA IX”, “AS CA IX” or “AS MN” refers to proteins and/or polypeptides encoded by the AS form of MN/CA9 mRNA.

MN proteins exhibit several interesting features: cell membrane localization, cell density dependent expression in HeLa cells, correlation with the tumorigenic phenotype of HeLa x fibroblast somatic cell hybrids, and expression in many human carcinomas among other tissues. MN protein can be found directly in tumor tissue sections but not in general in counterpart normal tissues (exceptions noted above as in normal gastric mucosa and gallbladder tissues). MN is also expressed sometimes in morphologically normal appearing areas of tissue specimens exhibiting dysplasia and/or malignancy. Taken together, those features indicate the involvement of MN in the regulation of cell proliferation, differentiation and/or transformation.

It can be appreciated that a protein or polypeptide produced by a neoplastic cell in vivo could be altered in sequence from that produced by a tumor cell in cell culture or by a transformed cell. Thus, MN proteins and/or polypeptides which have varying amino acid sequences including without limitation, amino acid substitutions, extensions, deletions, truncations and combinations thereof, fall within the scope of this invention. It can also be appreciated that a protein extant within body fluids is subject to degradative processes, such as, proteolytic processes; thus, MN proteins that are significantly truncated and MN polypeptides may be found in body fluids, such as, sera. The phrase “MN antigen” is used herein to encompass MN proteins and/or polypeptides.

It will further be appreciated that the amino acid sequence of MN proteins and polypeptides can be modified by genetic techniques. One or more amino acids can be deleted or substituted. Such amino acid changes may not cause any measurable change in the biological activity of the protein or polypeptide and result in proteins or polypeptides which are within the scope of this invention, as well as, MN muteins.

The MN proteins and polypeptides of this invention can be prepared in a variety of ways according to this invention, for example, recombinantly, synthetically or otherwise biologically, that is, by cleaving longer proteins and polypeptides enzymatically and/or chemically. A preferred method to prepare MN proteins is by a recombinant means.

### Recombinant Production of MN Proteins and Polypeptides

A representative method to prepare MN protein as, for example, the MN protein shown in FIG. 8 or fragments thereof, would be to insert the full-length or an appropriate fragment of MN cDNA into an appropriate expression vector. In Zavada et al., WO 93/18152, supra, production of a fusion protein GEX-3X-MN (now termed GST-MN) using a partial cDNA in the vector pGEX-3X (Pharmacia) is described. Nonglycosylated GST-MN (the MN fusion protein MN glutathione S-transferase) from XL1-Blue cells.

Zavada et al., WO 95/34650 describes the recombinant production of both a glycosylated MN protein expressed from insect cells and a nonglycosylated MN protein expressed from E. coli using the expression plasmid pEt-22b [Novagen Inc.; Madison, Wis. (USA)]. Recombinant baculovirus express vectors were used to infect insect cells. The glycosylated MN 20-19 protein was recombinantly produced in baculovirus-infected sf9 cells [Clontech; Palo Alto, Calif. (USA)].

### Preparation of MN-Specific Antibodies

The term “antibodies” is defined herein to include not only whole antibodies but also biologically active fragments of antibodies, preferably fragments containing the antigen binding regions. Further included in the definition of antibodies are bispecific antibodies that are specific for MN protein and to another tissue-specific antigen.

Antibodies useful according to the methods of the invention may be prepared by conventional methodology and/or by genetic engineering. Antibody fragments may be genetically engineered, preferably from the variable regions of the light and/or heavy chains (V_{H} and V_{L}), including the hypervariable regions, and still more preferably from both the V_{H} and V_{L} regions. For example, the term “antibodies” as used herein includes polyclonal and monoclonal antibodies and biologically active fragments thereof including among other possibilities “univalent” antibodies; Fab proteins including Fab′ and F(ab)₂ fragments whether covalently or non-covalently aggregated; light or heavy chains alone, preferably variable heavy and light chain regions (V_{H} and V_{L} regions), and more preferably including the hypervariable regions [otherwise known as the complementarity determining regions (CDRs) of the V_{H} and V_{L} regions]; F_{c} proteins; “hybrid” antibodies capable of binding more than one antigen; constant-variable region chimeras; “composite” immunoglobulins with heavy and light chains of different origins; “altered” antibodies with improved specificity and other characteristics as prepared by standard recombinant techniques and also oligonucleotide-directed mutagenesis techniques [Dalbadie-MacFarland et al., “Oligonucleotide-directed mutagenesis as a general and powerful method for studies of protein function,” PNAS USA 79: 6409 (1982)].

For many uses, particularly for pharmaceutical uses or for in vivo tracing, partially or more preferably fully humanized antibodies and/or biologically active antibody fragments may be found most particularly appropriate. Such humanized antibodies/antibody fragments can be prepared by methods well known in the art.

The antibodies useful according to this invention to identify MN proteins/polypeptides can be labeled in any conventional manner, for example, with enzymes such as horseradish peroxidase (HRP), fluorescent compounds, or with radioactive isotopes such as, ¹²⁵I, among many other labels. A preferred label, according to this invention is ¹²⁵I, and a preferred method of labeling the antibodies is by using chloramine-T [Hunter, W. M., “Radioimmunoassay,” In: Handbook of Experimental Immunology pp.14.1-14.40 (D. W. Weir ed.; Blackwell, Oxford/London/Edinburgh/Melbourne; 1978)]. Other exemplary labels may include, for example, allophycocyanin and phycoerythrin, among many other possibilities.

Zavada et al., WO 93/18152 and WO 95/34650 describe in detail methods to produce MN-specific antibodies, and detail steps of preparing representative MN-specific antibodies as the M75, MN7, MN9, and MN12 monoclonal antibodies.

### Epitopes

The affinity of a MAb to peptides containing an epitope depends on the context, e.g. on whether the peptide is a short sequence (4-6 aa), or whether such a short peptide is flanked by longer aa sequences on one or both sides, or whether in testing for an epitope, the peptides are in solution or immobilized on a surface. Therefore, it would be expected by ones of skill in the art that the representative epitopes described herein for the MN-specific MAbs would vary in the context of the use of those MAbs.

The term “corresponding to an epitope of an MN protein/polypeptide” will be understood to include the practical possibility that, in some instances, amino acid sequence variations of a naturally occurring protein or polypeptide may be antigenic and confer protective immunity against neoplastic disease and/or anti-tumorigenic effects. Possible sequence variations include, without limitation, amino acid substitutions, extensions, deletions, truncations, interpolations and combinations thereof. Such variations fall within the contemplated scope of the invention provided the protein or polypeptide containing them is immunogenic and antibodies elicited by such a polypeptide or protein cross-react with naturally occurring MN proteins and polypeptides to a sufficient extent to provide protective immunity and/or anti-tumorigenic activity when administered as a vaccine.

### Immunodominant Epitopes in PG Domain and in Neighboring Regions

As indicated above, the extracellular domain of the full-length CA IX comprises the PG and CA domains as well as some spacer or perhaps hinge regions. The CA IX immunodominant epitopes are primarily in the PG region at about aa 53-111 (SEQ ID NO: 8) or at about aa 52-125 (SEQ ID NO: 81), preferably now considered to be at about aa 52-125 (SEQ ID NO: 81). The immunodominant epitopes of CA IX may be located in regions neighboring the PG region. For example, the epitope for aa 36-51 (SEQ ID NO: 21) would be considered an immunodominant epitope.

The main CA IX immunodominant epitope is that for the M75 mab. The M75 monoclonal antibody is considered to be directed to an immunodominant epitope in the N-terminal, proteoglycan-like (PG) region of CA IX. Alignment of amino acid sequences illustrates significant homology between the MN/CA IX protein PG region (aa 53-111) [SEQ ID NO: 8] and the human aggrecan (aa 781-839) [SEQ ID NO: 10]. The epitope of M75 has been identified as amino acid sequence PGEEDLP (SEQ ID NO: 11), which is 4x identically repeated in the N-terminal PG region of CA IX [Zavada et al. (2000)]. Closely related epitopes to which the M75 mab may also bind, which are also exemplary of immunodominant epitopes include, for example, the immunodominant 6× tandem repeat that can be found at amino acids (aa) 61-96 (SEQ ID NO. 12) of FIG. 8A-8C, showing the predicted CA IX amino acid sequence. Variations of the immunodominant tandem repeat epitopes within the PG domain include GEEDLP (SEQ ID NO: 13) (aa 61-66, aa 79-84, aa 85-90 and aa 91-96), EEDL (SEQ ID NO: 14) (aa 62-65, aa 80-83, aa 86-89, aa 92-95), EEDLP (SEQ ID NO: 15) (aa 62-66, aa 80-84, aa 86-90, aa 92-96), EDLPSE (SEQ ID NO: 16) (aa 63-68), EEDLPSE (SEQ ID NO: 17) (aa 62-68), DLPGEE (SEQ ID NO: 18) (aa 82-87, aa 88-98), EEDLPS (SEQ ID NO: 19) (aa 62-67) and GEDDPL (SEQ ID NO: 20) (aa 55-60). Other immunodominant epitopes could include, for example, aa 68-91 (SEQ ID NO: 22).

The monoclonal antibodies MN9 and MN12 are considered to be directed to immunodominant epitopes within the N-terminal PG region SEQ ID NOS: 19-20, respectively. The MN7 monoclonal antibody could be directed to an immunodominant epitope neighboring the PG region at aa 127-147 (SEQ ID NO: 23) of FIG. 8A-8C.

An epitope considered to be preferred within the CA domain (SEQ ID NO: 9) is from about aa 279-291 (SEQ ID NO: 67). An epitope considered to be preferred within the intracellular domain (IC domain) (SEQ ID NO: 7) is from about aa 435450 (SEQ ID NO: 68).

SEQ ID NO: 69 (aa 166-397 of FIG. 8A-8C) is considered to be an important antigenic component of the CA domain. There are several antigenic sites within the CA domain. There are four groups of the CA IX-specific monoclonal antibodies that have been prepared in CA IX-deficient mice such that they are directed to the CA domain; three of those groups are within SEQ ID NO: 69. Antigenic site(s) may be partly located also on the amino acids 135-166 (SEQ ID NO: 84). An exemplary preferred MN-specific antibody that specifically binds the carbonic anhydrase domain of MN protein is the V/10 Mab, which is produced by the hybridoma VU-V/10, deposited at BCCM™/LMBP in Ghent, Belgium under Accession No. LMBP 6009CB.

### Assays

### Assays to Screen for AS and FL MN/CA IX Expression in Tissues

The methods may comprise screening for AS and/or FL MN/CA9 gene expression product(s), if any, present in a sample taken from a patient diagnosed with a preneoplastic/neoplastic disease; the MN/CA9 gene expression product(s) can be AS or FL form(s) of MN protein, MN polypeptide, mRNA encoding a MN protein or polypeptide, a cDNA corresponding to an mRNA encoding a MN protein or polypeptide, or the like.

Many formats can be adapted for use with the methods of the present invention. The detection and quantitation of AS and/or FL MN mRNA can be performed, for example, by a nucleic acid amplification method, such as the use of PCR, RT-PCR, real-time PCR or quantitative real-time RT-PCR, or may be performed by the use of a microarray chip. The detection and quantitation of AS and/or FL MN protein or MN polypeptide can be performed by Western blots, enxyme-linked immunosorbent assays, radioimmunoassays, competition immunoassays, dual antibody sandwich assays, immunohistochemical staining assays, agglutination assays, fluorescent immunoassays, immunoelectron and scanning microscopy using immunogold, among other assays commonly known in the art. The detection of MN AS and/or FL gene expression products in such assays can be adapted by conventional methods known in the art.

Nucleic Acid Probes and/or Primers

Nucleic acid probes and/or of this invention are those comprising sequences that are complementary or substantially complementary to the MN cDNA sequence shown in FIG. 8 [SEQ ID NO: 1] or to other MN gene sequences, such as, the complete genomic sequence of FIG. 9A-F [SEQ ID NO: 3]. The phrase “substantially complementary” is defined herein to have the meaning as it is well understood in the art and, thus, used in the context of standard hybridization conditions. The stringency of hybridization conditions can be adjusted to control the precision of complementarity. Two nucleic acids are, for example, substantially complementary to each other, if they hybridize to each other under stringent hybridization conditions. As indicated above, only very closely related nt sequences having a homology of at least 80-90%, preferably at least 90%, would hybridize to each other under stringent conditions.

Particularly preferred probes and/or primers for use in the invention are probes and/or primers that differentiate between full-length [FL] and alternatively-spliced [AS] MN/CA9 mRNA expression. Many recent articles provide general information regarding alternative splicing in cancer, and specific information regarding the design of specific probes and/or primers used to detect mRNA variants expressed by cancer-related genes, from which probes and/or primers could be designed to detect AS and/or FL CA9 mRNA variants [See, for example, Matlin et al., Nat Rev Mol Cell Biol. 6: 386-398 (2005); Venables J P, BioEssays, 28: 378-386 (2006); Skotheim and Nees, Int J Biochem Cell Biol. 39(7-8): 1432-1449 (2007); Srebrow and Komnblihit, J Cell Sci., 119(Pt 13): 2635-2641 (2006); Gothie et al., J Biol Chem. 275: 6922-6927 (2000); Robinson et al., J Cell Sci., 114: 853-865 (2001); He et al., Oncogene, 25: 2192-2202 (2006); Roy et al., Nucleic Acids Res., 33(16): 5026-5033 (2005); Taconelli et al., Cancer Cell, 6: 347-360 (2004)]. In one method, at least one probe or primer used to detect only FL CA9 mRNA would be derived wholly or in part from inside a region deleted in AS CA9 mRNA, whereas at least one probe or primer used to detect only AS CA9 mRNA would be derived from the alternative splicing-generated junction. For example, a human FL MN/CA9-specific probe/primer could comprise a nucleic acid which binds with adequate specificity, preferably specifically, to exon 8 or exon 9 of the human MN/CA9 gene, or binds with adequate specificity, preferably specifically, to the splice junction of exons 7 and 8, the splice junction of exons 8 and 9, or the splice junction of exons 9 and 10 of the human MN/CA9 gene; or could comprise any nucleic acid sufficiently homologous to bind with adequate specificity, preferably specifically to any of those sequences. Similarly, a human AS MN/CA9-specific probe/primer could comprise a nucleic acid which binds with adequate specificity, preferably specifically, to the splice junction of exons 7 and 10 of the human MN/CA9 gene; or could comprise any nucleic acid sufficiently homologous to bind with adequate specificity, preferably specifically, to that splice junction. Alternatively, probes and/or primers could be used to detect both FL and AS CA9 mRNA, and the FL and AS mRNA products differentiated by their length, e.g., on a gel.

### Methods of Cancer Therapy Based on MN Alternative Splicing Variants

A number of articles discuss cancer therapies based on alternative splicing variants of cancer-related genes, and provide strategies for the design of oligonucleotides used for antisense and RNA interference therapies, among other therapies [e.g., Garcia-Blanco, Curr Opin Mol Ther., 7(5): 476-482 (2005); Wilton and Fletcher, Curr Gene Ther., 5(5): 467483 (2005); Pajares et al., Lancet Oncol., 8(4):349-357 (2007); Xing Y., Front Biosci., 12: 4034-4041 (2007)]. For example, ones of ordinary skill in the art could determine appropriate antisense nucleic acid sequences, preferably antisense oligonucleotides, specific to the human FL CA9 mRNA, and not the human AS CA9 mRNA, from the nucleic acid sequences of SEQ ID NOS: 1 and 108, respectively.

In addition to the entire AS MN/CA IX expressed by the AS form of CA9 mRNA, one of skill in the art would expect that isolated AS MN/CA IX protein or polypeptide fragments would have the ability to interfere with FL MN/CA IX activity. Accordingly, any protein or polypeptide derived from AS MN/CA IX that interferes with the activity of FL MN/CA IX is considered within the scope of therapeutic methods of the invention.

### MN RNA Interference (MN RNAI)

Inhibition of the expression of the MN gene can be carried out, for example, by applying an RNA interference effect on the expression of the MN gene. RNA interference is a method for inhibiting the expression of a gene by using RNA, as has been reported in recent years [Elbashir et al., Nature. 411: 494-498 (2001)]. More specifically, the expression of the MN gene can be inhibited by using one or more oligonucleotides that exhibit an RNA interference effect on the expression of a particular mRNA splice variant (such as the FL splice variant) of the MN gene.

Inhibition of the expression of an mRNA splice variant of the MN gene can be carried out by transfecting a cell with a vector containing a fragment of the cDNA or with the complementary RNA thereof. Accordingly, an agent for inhibiting an MN splice variant comprising the said oligonucleotide(s) is also included in the scope of the present invention. The agent for inhibiting an MN mRNA splice variant may contain one kind of oligonucleotide, or may contain two or more kinds of oligonucleotide. The said oligonucleotide exhibiting an RNA interference effect can be obtained from oligonucleotides that are designed on the basis of the nucleotide sequence of the AS and/or FL mRNA variants of the MN gene, by selecting oligonucleotides that specifically silence the expression of the FL mRNA variant using an MN gene expression system.

### MN Gene Therapy Vectors

For inhibiting the expression of FL MN/CA IX using an oligonucleotide, it is possible to introduce the oligonucleotide into the targeted cell by use of gene therapy. The gene therapy can be performed by using a known method. For example, either a non-viral transfection, comprising administering the oligonucleotide directly by injection, or a transfection using a virus vector can be used. A preferred method for non-viral transfection comprises administering a phospholipid vesicle such as a liposome that contains the oligonucleotide, as well as a method comprising administering the oligonucleotide directly by injection. A preferred vector used for a transfection is a virus vector, more preferably a DNA virus vector such as a retrovirus vector, an adenovirus vector, an adeno-associated virus vector and a vaccinia virus vector, or a RNA virus vector.

### Materials and Methods

### Cell Culture, Tissues and Antibodies

Mouse NIH 3T3 fibroblasts, canine MDCK epithelial cells, human tumor cell lines CAKI-1 and ACHN derived from kidney carcinoma, as well as Caski, SiHa, HeLa, and C33a lines from cervical carcinoma were cultivated in DMEM supplemented with 10% FCS (BioWhittaker, Verviers, Belgium) and 40 μg/ml gentamicin (Lek Slovenia) in a humidified atmosphere with 5% CO₂ at 37° C. Hypoxic treatments were performed in an anaerobic workstation (Ruskin Technologies, Bridgend, UK) in 2% O₂, 5% CO₂, 10% H₂ and 83% N₂ at 37° C.

HeLa spheroids were pre-formed from 400 cells per 20 μl of culture medium in drops hanging on the lid of tissue culture dish for three days at 37° C. The resulting cell aggregates were transferred to Petri dish with a non-adherent surface and cultivated in suspension for additional 11 days, with the medium exchange every third day. The spheroids were examined with a Nikon E400 microscope and photographed with a Nikon Coolpix 990 camera.

Human tissues were selected from the collection described previously (Kivela et al, 2005). Mouse tissues were dissected from BALB/c mouse sacrificed by cervical dislocation. The tissues were stored at −80° C. until used for RNA isolation and/or protein extraction.

M75 and V/10 mouse MAbs specific for the human MN/CA IX protein were characterized earlier (Pastorekova et al, 1993, Zatovicova et al, 2003). Secondary anti-mouse peroxidase-conjugated antibodies and anti-rabbit antibodies conjugated with horse-radish peroxidase were from Sevapharma (Prague, Czech Republic). Anti-mouse FITC-conjugated antibodies were from Vector Laboratories (Burlingame, Calif.). Alexa 488-conjugated anti-rabbit secondary antibodies were obtained from Advanced Targeting Systems (San Diego, Calif.).

### Immunofluorescence

Immunofluorescence was performed as described previously (Svastova et al, 2004). Cells grown on glass coverslips were rinsed twice with ice-cold PBS and fixed with cold methanol for 5 min at −20° C. The coverslips were incubated with PBS containing 1% BSA for 30 min at 37° C., and then with undiluted hybridoma medium containing M75 MAb or rabbit polyclonal serum against mCA IX diluted 1:1000. Antibodies against the mouse CA IX protein were described elsewhere (Gut et al, 2002). Incubation with primary antibody was performed for 1 h in a humidified chamber at 37° C. The coverslips were washed three times with PBS containing 0.02% Tween-20 for 10 min and then treated with fluorescein-conjugated anti-mouse secondary antibodies diluted 1:300 in 0.5% BSA in PBS for 1 h at 37° C. or with anti-rabbit Alexa 488-conjugated secondary antibody diluted 1:1000 in 0.5% BSA in PBS. After rinsing three times with PBS for 10 min, the coverslips were mounted onto microscope slides with mounting medium (Calbiochem, Cambridge, MA) and then examined with a Nikon E400 microscope and photographed with Nikon Coolpix 990 camera.

### Expression Plasmids

The eukaryotic expression plasmid pSG5C-mAS encoding the mouse splicing variant was generated by inverse PCR from pSG5C-Car9 plasmid containing the mouse CA9 cDNA. The forward primer was designed to the start of exon 9 (m9S, 5′-TCCATGTGMTTCCTGCTTCACTG-3′) [SEQ ID NO: 102] and the reverse primer was specific to the end of exon 6 (m6A, 5′-CTTCCTCCGAGATTTCTTCCAAAT-3′) [SEQ ID NO: 103]. Similarly, the eukaryotic expression plasmid pSG5C-AS encoding the human splicing variant was generated by inverse PCR from the pSG5C-MN/CA9 expression plasmid (Pastorek et al, 1994) that contains a full-length human CA9 cDNA (GenBank # X66839) using the primers to exons 10 and 7. The forward primer (h10S, 5′-GTGACATCCTAGCCCTGGTTTTT-3′) [SEQ ID NO: 104] was specific to the start of exon 10 and the reverse primer (h7A, 5′-CTGCTTAGCACTCAGCATCA CTG-3′) [SEQ ID NO: 105] was specific to the end of exon 7. The same h7A and h10S primers were used for the preparation of a bacterial expression vector pGEX-3X-AS encoding a GST-fused splice variant of the human CA IX protein, from the primary plasmid construct pGEX-3X-CA9 coding for the full-length CA IX protein without the signal peptide. PCR amplifications were performed using a Phusion polymerase (Finnzymes, Espoo, Finland). PCR reactions consisted of an initial denaturing at 98° C. for 30 s, 32 cycles of denaturing at 98° C. for 10 s, annealing at 64° C. for 30 s, extension at 72° C. for 1 min 40 s, and final extension for 5 min at 72° C. PCR products were gel purified, treated with T4 polynucleotide kinase and ligated with T4 DNA ligase (Invitrogen, Carlsbad, USA). All constructs were verified by sequencing. The construct coding for GST-PGCA fusion protein containing the extracellular part of the human CA IX was described earlier (Zatovicova et al, 2003). Table 2 below provides the sequences of primers used in the Examples.

TABLE 2

| | | | | |
|---|---|---|---|---|
| Primer | | | | SEQ |
| desig- | | | ID | |
| nation | Position | Sequence (5′-3′) | NO | |

| | | | |
|---|---|---|---|
| mβ- | 768-787 | GTTGGCATAGAGGTCTTACG | 85 |
| actin S | | | |
| mβ- | 968-948 | GCCGCATCCTCTTCCTCCCT | 86 |
| actin A | | | |
| M6S | 794-814 | GGAGGCCTGGCAGTTTTGGCT | 87 |
| M11A | 1358-1336 | CTCCAGTTTCTGTCATCTCTGCC | 88 |
| M8S | 1156-1175 | CCCTGCTGCAGAGGATAGCA | 89 |
| M10A | 1312-1293 | GGTCCCACTTCTGTGCCTGT | 90 |
| M6/9S | 883-893/ | CTCGGAGGAAG/TCCATGTGAA | 91 |
| | 1188-1194 | | |
| M10A | 1312-1293 | GGTCCCACTTCTGTGCCTGT | 92 |
| hβ- | 414-433 | CCAACCGCGGGAAGATGACC | 93 |
| actin S | | | |
| hβ- | 649-629 | GATCTTCATGAGGTAGTCAGT | 94 |
| actin A | | | |
| h1S | 412-433 | GAACCCCAGAATAATGCCCACA | 95 |
| h6A | 924-945 | TCGCTTGGAAGAAATCGCTGAG | 96 |
| h6S | 915-937 | GTTGCTGTCTCGCTTGGAAGAAA | 97 |
| h11A | 1392-1372 | GCGGTAGCTCACACCCCCTTT | 98 |
| h7S | 980-1001 | TATCTGCACTCCTGCCCTCTG | 99 |
| h8A | 1133-1155 | CACAGGGTGTCAGAGAGGGTGT | 100 |
| h10/7A | 1291-1279/ | CTAGGATGTCAC/CTGCTTAGCACTC | 101 |
| | 1106-1095 | | |

### Transfection

The cells were plated in 60 mm Petri dishes to reach approximately 70% density on the next day. Transfection was performed with 2 μg of the pSG5C-hAS and pSG5C-mAS plasmids encoding the splicing variants of the human and mouse CA IX, respectively, together with 200 ng of pSV2neo plasmid. Transfection was performed with 2 μg of the pSG5C-hAS and pSG5C-mAS plasmids encoding the splicing variants of the human and mouse CA IX, respectively, together with 200 ng of pSV2neo plasmid using the Gene Porter II transfection reagent (Genlantis, San Diego, Calif.). The transfected cells were subjected to selection using G418 (Invitrogen) at a concentration of 900 μg/ml for HeLa cells, and 500 μg/ml for MDCK cells. The resistant colonies were cloned, tested for expression of the splicing variant by immunoblotting and expanded.

### Binding of Fluorescent CA Inhibitor

The fluorescent CA inhibitor (FITC-CAI) was obtained by reaction of homosulfanilamide with fluorescein isothiocyanate and showed a K_{I} value of 24 nM towards CA IX (Svastova et al, 2004, Cecchi et al, 2005). The inhibitor was dissolved in PBS with 20% DMSO at 100 mM concentration and diluted in a culture medium to a final 1 mM concentration just before the addition to cells. The MDCK-CA IX cells (Svastova et al, 2004) were plated at a density of 4×10⁵ cells per 3.5 cm dish in the medium containing the conditioned medium from MDCK-AS transfectants that secrete the human AS variant. Control cells were incubated in the absence of secreted AS. After 24 h incubation, equivalent fresh media were replenished, FITC-CAI was added to cells, the cells were transferred to hypoxic workstation and the binding was allowed for additional 48 h. Parallel samples were incubated in normoxia. At the end, the cells were washed five times with PBS and viewed by a Nikon E400 epifluorescence microscope. Intensity of the fluorescence was evaluated from acquired images using the Scion Image Beta 4.02 software (Scion Corporation, Frederick, Md.) and relative FITC-CAI binding was expressed in per cent.

### Protein Extraction

Proteins were extracted from cell monolayer or tissue homogenate with RIPA buffer as described previously (Svastova et al, 2004). Proteins were extracted from cell monolayer or tissue homogenate with RIPA buffer (1% Triton X-100, 0.1% sodium deoxycholate, 1 mM phenylmethylsulfonyl fluoride in PBS) containing inhibitors of proteases Complete mini (Roche Applied Science, Mannhein, Germany) for 30 min on ice. The extracts were centrifuged for 15 min at 13000 rpm and total protein concentrations were determined by BCA assay (Pierce, Rockford, Ill.) according to the manufacturer's instructions. The extracts (aliquots containing 30-50 μg) of total proteins were separated in 10% and 8% SDS-PAGE in Laemmli sample buffer with 2-mercaptoethanol (reducing conditions) or without 2-mercaptoethanol (non-reducing conditions).

### Immunoprecipitation and Immunoblotting

The samples for detection of extracellular human AS were prepared from the culture medium of AS-transfected cells incubated without FCS under hypoxia and normoxia for 24 h. One fourth (500 μl) of the culture medium was 10-times concentrated and separated in SDS-PAGE. For immunoprecipitation, CA IX-specific MAbs in 1 ml of hybridoma medium were bound to 25 μl 50% suspension of Protein-A Sepharose (Pharmacia, Uppsala, Sweden) for 2 h at RT. Cell extract (200 μl) was pre-cleared with 20 μl of 50% suspension of Protein-A Sepharose and then added to the bound MAbs. Immunocomplexes collected on Protein-A Sepharose were washed, boiled and subjected to SDS-PAGE and immunoblofting as described previously (Zatovicova et al, 2003). Proteins were separated in SDS-PAGE and blotted onto the polyvinylidene fluoride (PVDF) membrane (Immobilon™-P, Millipore, Billerica, Mass.). The membrane was treated with the blocking buffer containing 5% non-fat milk in PBS with 0.2% Nonidet P40 for 1 h and then incubated for 1 h with the primary antibody diluted in the blocking buffer (either M75 monoclonal antibody in hybridoma medium diluted 1:2, or rabbit anti-mouse CA IX polyclonal antibody diluted 1 :1000). After treatment, the membrane was thoroughly washed in PBS with 0.2% Nonidet P40 for 45 min, incubated for 1 h with the swine anti-mouse or anti-rabbit secondary antibodies conjugated with horseradish peroxidase (Sevapharma) diluted 1:7500 and 1:5000 in the blocking buffer. The membranes were washed in PBS with 0.2% Nonidet P40 (Sigma, St Louis, Mo.) and developed with ECL detection system.

For isolation of membrane and sub-membrane proteins and analysis of oligomers, the cells were washed with PBS and incubated with RIPA extraction buffer for 30 s on ice. RIPA buffer with proteins was aspirated and fresh RIPA buffer was added to the cells. The remaining proteins were then extracted for 15 min on ice. Oligomers were first immunoprecipitated from HeLa-AS extract using the CA IX-specific MAbs V/10 (recognizes FL but not AS) or M75 (recognizes both variants). Components of the precipitated oligomers were resolved in reducing SDS-PAGE, blotted and visualized using the peroxidase-labelled M75.

### Reverse Transcription PCR

Total RNA was isolated either from cells or from tissues using InstaPure reagent (Eurogentec, Seraing, Belgium). Reverse transcription was performed with M-MuLV reverse transcriptase (Finnzymes, Oy, Finland) using random heptameric primers (400 ng/μl). The mixture of 5 μg of total RNA and random primers (400 ng/μl) was heated for 10 min at 70° C., cooled quickly on ice and supplemented with 0.5 mM dNTPs (Finnzymes), reverse transcriptase buffer containing 6 mM MgCl₂, 40 mM KCl, 1 mM DTT, 0.1 mg/ml BSA and 50 mM Tris-HCl, pH 8.3. The mixture in a final volume of 24 μl was further supplemented with 200 U of reverse transcriptase M-MuLV, incubated for 1 h at 42° C., heated for 15 min at 70° C. and stored at −80° C. until used.

PCR was performed with Dynazyme EXT polymerase (Finnzymes) with the primers listed in Table 2 (supra). Resulting PCR fragments were run on 1.5% agarose gels. The protocol of PCR consisted of 94° C. for 3 min followed by 30 cycles of: denaturing at 94° C. for 30 s, annealing for 40 s (temperature depended on sets of primers) and extension at 72° C. for 40 s, followed by a final extension at 72° C. for 5 min. The PCR products were purified and sequenced using automatic sequencer from Applied Biosystems ABI 3100 (Foster City, USA).

The following examples are for purposes of illustration only and are not meant to limit the invention in any way.

### Example 1

### Identification, structure and Expression of a Mouse Splice Variant of CA IX

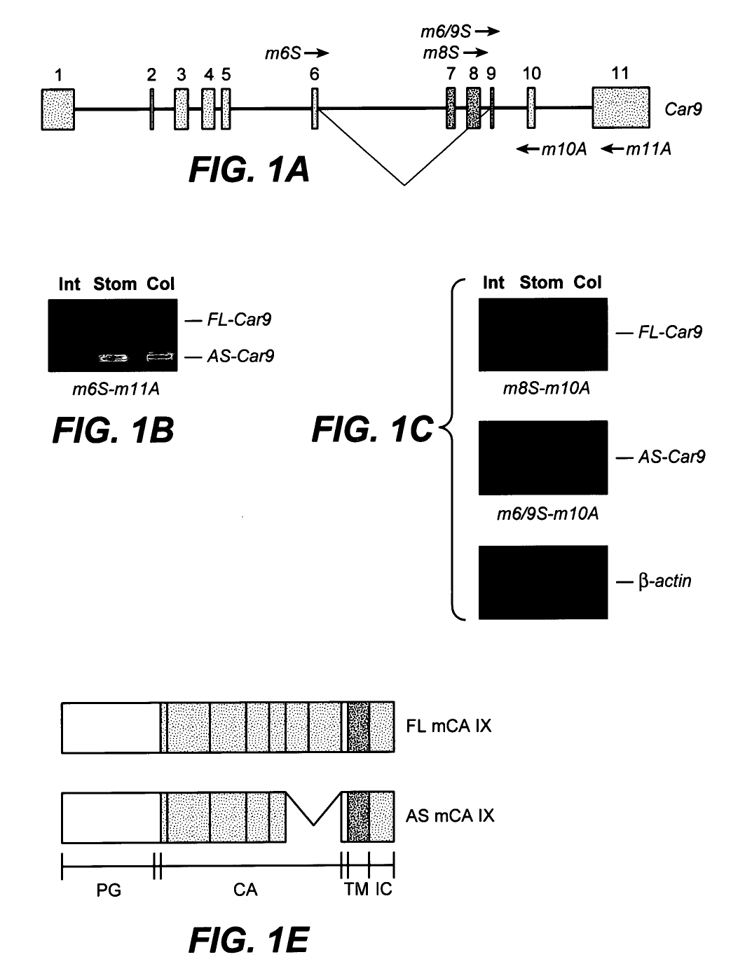

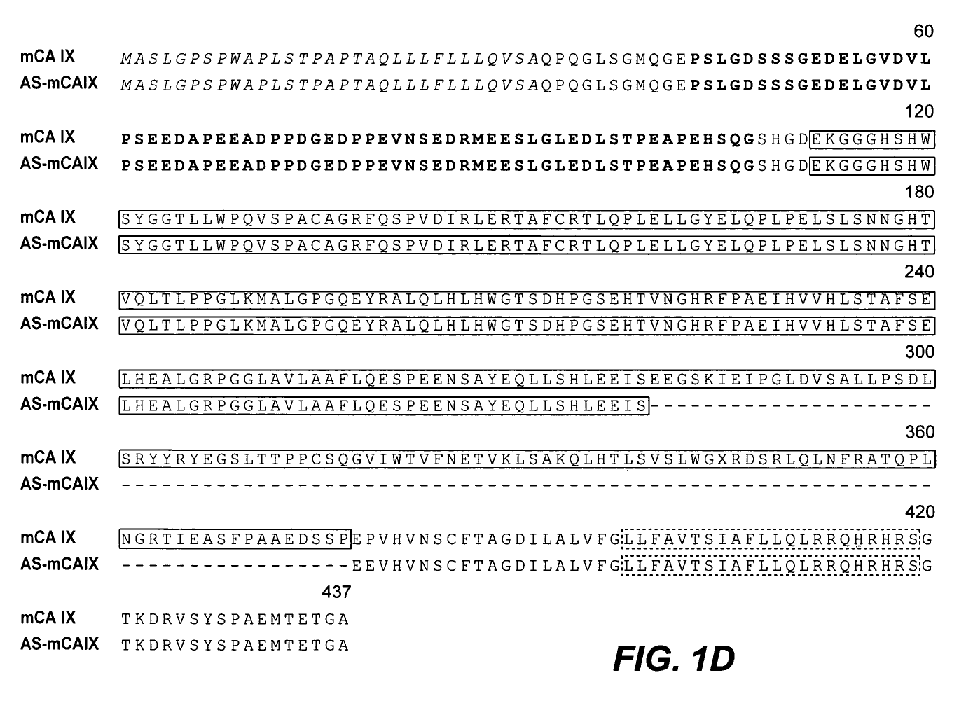

Earlier reverse transcription (RT) PCR data related to expression of Car9 mRNA in the mouse tissues were based on amplification of exons 1-6. However, RT PCR analysis of Car9 mRNA using the primers m6S and m11A to amplify the region spanning exons 6-11 revealed the presence of two amplification products—one PCR product of expected size and one smaller product (FIG. 1A,B). Sequencing of this smaller PCR product proved its Car9 specificity and showed that it represents an alternative splicing (AS) variant of the mouse Car9 mRNA, which is lacking the exons 7 and 8. This mouse AS variant was found in all three analyzed tissues—the stomach, small intestine and colon (FIG. 1B). Individual RT PCR amplification of the wild type and AS variant of Car9 using the corresponding pairs of primers (m8S-m10A for wt and m6/9S-m10A for AS) confirmed simultaneous presence of both products in the analyzed tissues (FIG. 1C).

Computer analysis of AS variant sequence showed that the deduced protein is by about 6 kDa smaller than the full-length mouse CA IX and its predicted molecular weight is 48 kDa. The splicing variant has a coding capacity for the protein with deleted amino acids 335-379, which lacks the C-terminal part of the catalytic (CA) domain and the region upstream of the transmembrane anchor, whereas the transmembrane and intracytoplasmic domains remain intact (FIG. 1D,E).

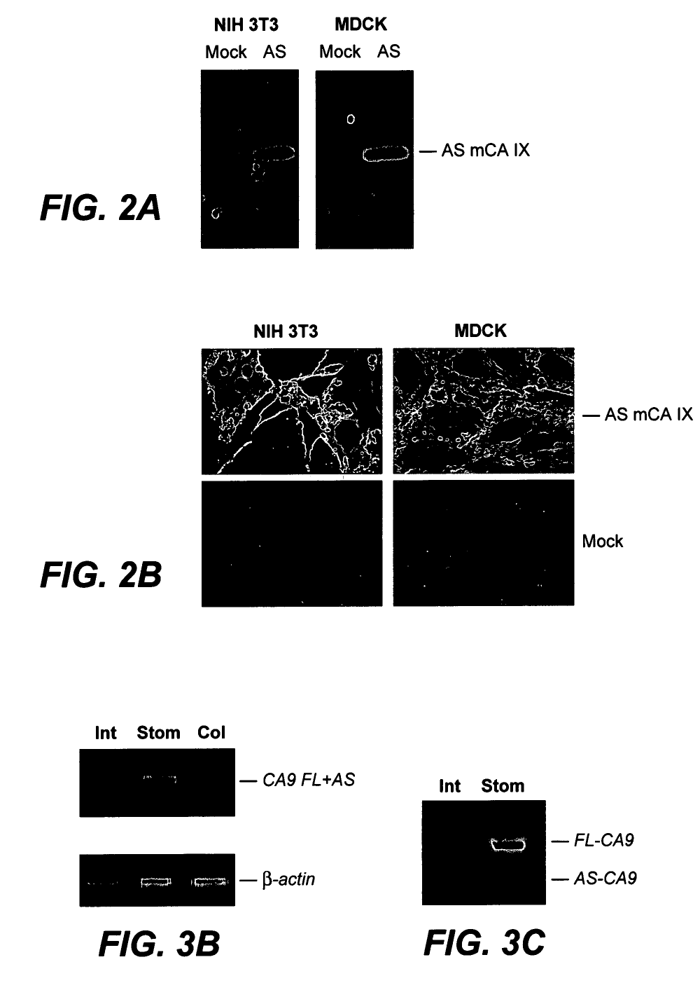

To study a subcellular localization of the mouse AS CA IX variant, the inventors cloned AS Car9 cDNA into pSG5C expression plasmid and used it for the generation of permanently transfected cell lines. AS variant has been overexpressed in the mouse NIH3T3 fibroblasts and canine MDCK epithelial cells that do not contain an endogenous CA IX protein. Both transfected cell lines were examined by immunoblotting and immunofluorescence using polyclonal anti-mouse CA IX antibodies (Gut et al, 2002). A single band of approximately 48 kDa was detected in the cell extracts of transfectants, corresponding well with the computer-predicted molecular weight of the mouse AS CA IX protein (FIG. 2A). The transfected cells exhibited clear cytoplasmic staining, suggesting that the mouse AS variant is localized in the cytosol (FIG. 2B).

### Example 2

### Identification and structure of a Human splice Variant of CA IX

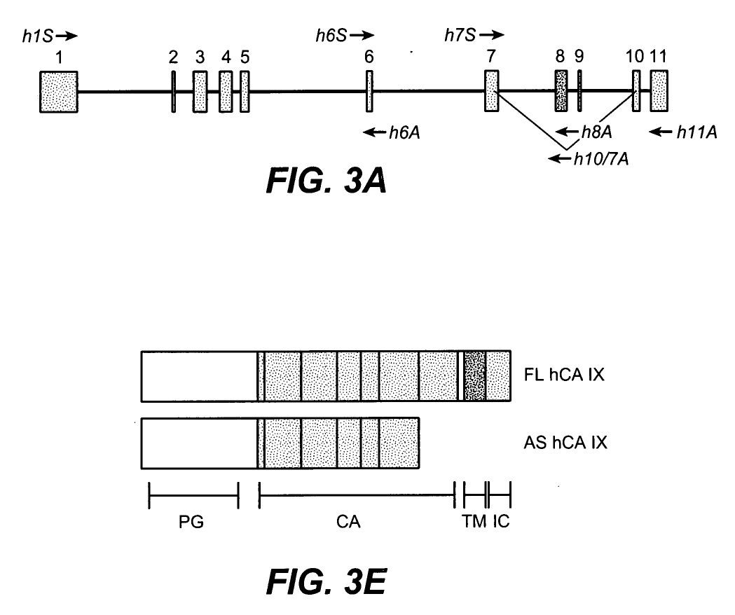

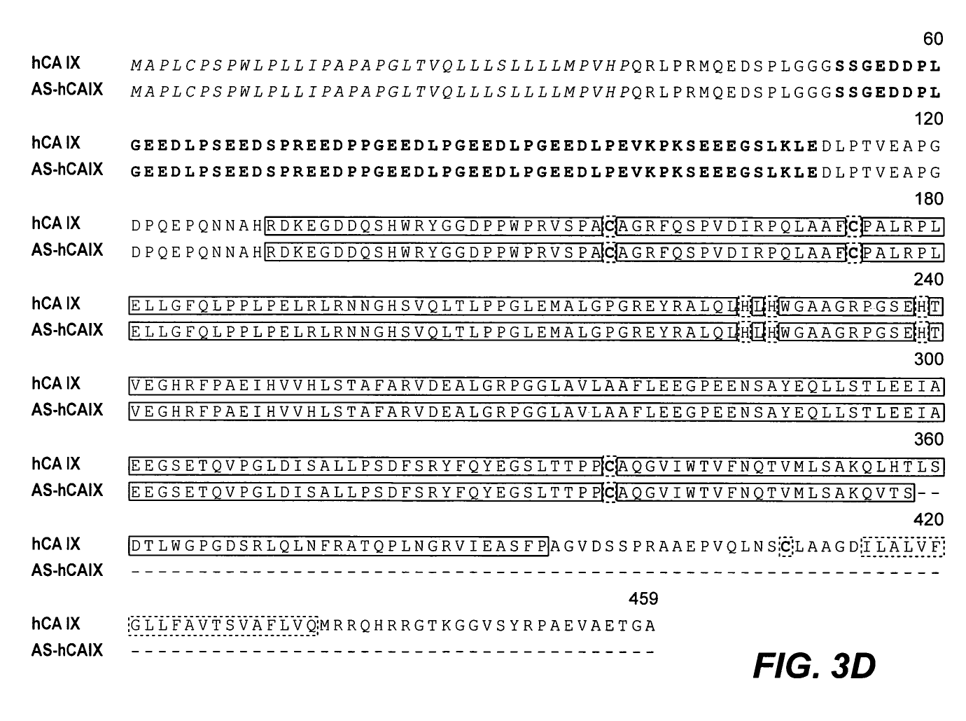

To search for the AS CA9 mRNA in human tissues and cell lines, the inventors designed a set of primers that covered the entire human CA9 mRNA (FIG. 3A). These were employed in RT-PCR on cDNA templates reverse-transcribed from mRNAs isolated from the human stomach and small intestine. Using the primers designed against exons 1 and 6 the inventors detected only a predicted PCR product (FIG. 3B). However, the primers to exons 6 and 11 generated two PCR amplicons—a more abundant longer product and a much less abundant shorter product (FIG. 3C). Sequence analysis of the shorter product confirmed that it corresponds to a human AS variant of CA9 mRNA. The splicing led to a deletion of exons 8 and 9.

Computer-predicted human AS CA IX protein is lacking the amino acids 356412 and its deduced molecular weight is about 43 kDa compared to a predicted size of 49 kDa for the full-length (FL) CA IX. The deletion eliminated 35 amino acids from the C-terminal part of the catalytic CA domain and 21 amino acids localized between the CA domain and the transmembrane region, which include Cys⁴⁰⁹ that appears to participate in the formation of intermolecular S-S bonds (FIG. 3D). Due to a frameshift-generated stop codon at position 1119 bp in AS mRNA (in FL CA9 mRNA, the stop codon is at position 1142 bp), the AS protein is truncated and contains neither the transmembrane nor the intracytoplasmic domains (FIG. 3E).

### Example 3

### Expression of Human AS CA IX in tumor Cell Lines and Tissues

To facilitate a separate detection of the FL and AS variants of CA9 mRNA, the inventors utilized primers that allowed for their individual amplification. The design was based on placing one FL-specific primer inside the deleted region and one AS-specific primer on the alternative splicing-generated junction (FIG. 3A).

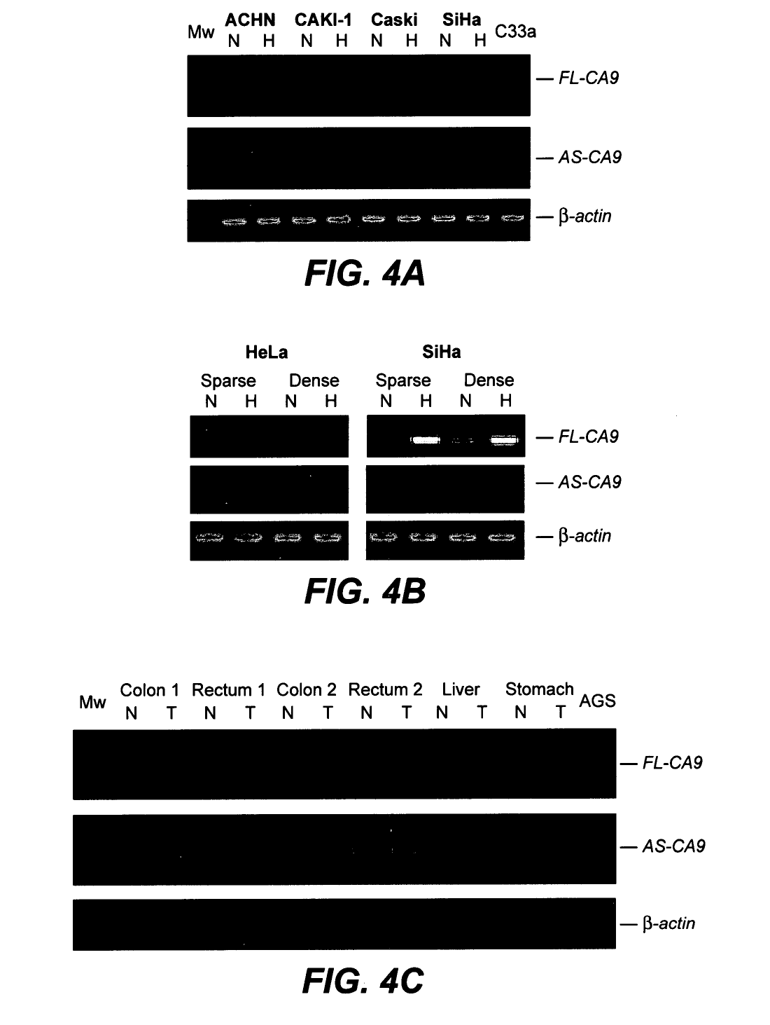

First, the inventors analyzed the presence of the AS variant in the human cancer cell lines exposed to hypoxia (2%) and normoxia (21%). The AS variant was detected in all examined cell lines and displayed similar levels under both normoxia and hypoxia (FIG. 4A). This was in contrast to FL CA9 mRNA, which was clearly hypoxia-inducible and showed considerably increased levels, namely in ACHN cells derived from kidney carcinoma and in Caski and SiHa cells derived from cervical carcinoma, whereas CAKI-1 cells expressed only a very low level of FL CA9 (FIG. 4A). No FL CA9-specific signal was observed in C33a cervical carcinoma cells that lack the CA9 gene (Lieskovska et al, 1999).

Previous studies have shown a density-induced expression of the FL CA IX that was associated with pericellular hypoxia (Kaluz et al, 2002). To see whether expression of the AS variant is density-dependent, the inventors used HeLa and SiHa cells cultivated in sparse culture (plated at 1×10⁴ cells per cm²) and dense culture (8×10⁴ cells per cm²), respectively, for 24 h. The dense cells clearly showed normoxic expression of the FL CA9 mRNA, although its level was lower that in the hypoxic cells. No remarkable differences were observed between the cells cultivated in sparse and dense monolayer with regard to level of the AS variant (FIG. 4B).

Finally, the inventors analyzed the AS expression in normal versus malignant human tissues, including the stomach, colon, rectum and liver. RT-PCR revealed the presence of the AS variant in all examined tissues (FIG. 4C). In accord with the previous studies, FL transcript was found only in the normal stomach and in tumors derived from colon and rectum (Saarnio et al, 1998, Kivela et al, 2005).

### Example 4

### Localization and Basic Characteristics of the Human AS Variant of CA IX

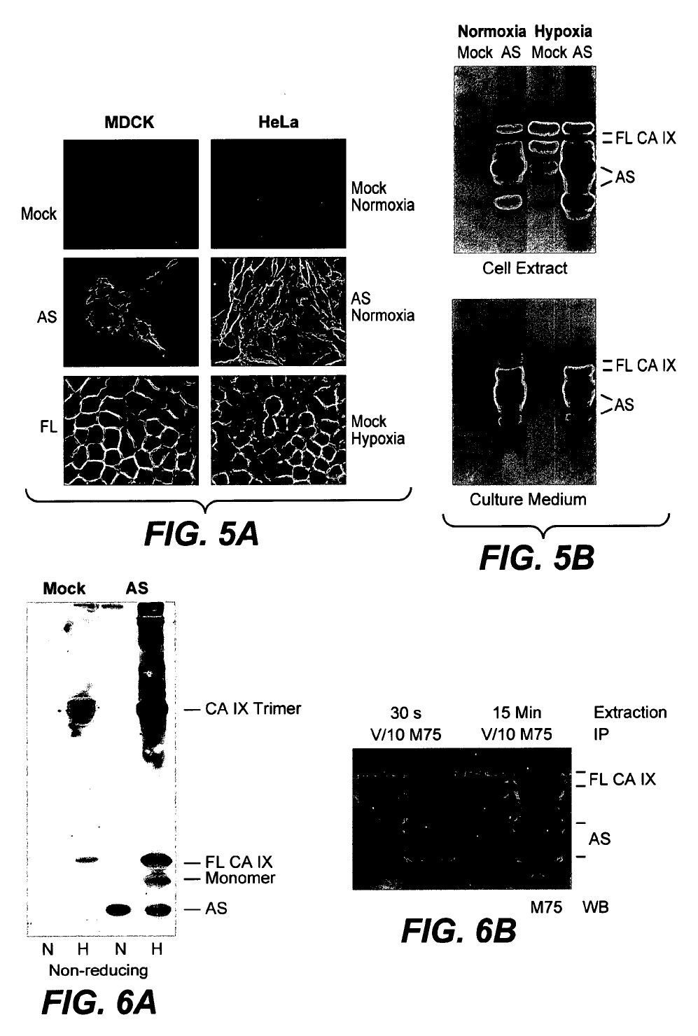

To perform a basic characterization of the AS variant of CA IX, the inventors generated stable transfectants with ectopic expression of the human AS protein. The human AS cDNA was transfected into CA IX-negative MDCK cells as well as to human HeLa cervical carcinoma cells that naturally express FL CA IX in response to density and hypoxia. In accord with the computer analysis that predicted splicing- and frameshift-mediated removal of TM and IC regions, the AS CA IX protein was not confined to the plasma membrane, but showed intracellular localization in both MDCK cells and in normoxic HeLa cells (FIG. 5A). This was clearly contrasting with the cell surface localization of the FL CA IX in the transfected MDCK cells and in the mock-transfected HeLa cells exposed to hypoxia (2% O₂).

The transfected HeLa-AS cells exhibited in immunoblotting two bands of approximately 43/47 kDa corresponding to the AS CA IX and additional two bands of 54/58 K corresponding to the hypoxia-induced FL CA IX (FIG. 5B). Because of the complete absence of the transmembrane and intracellular domains from the AS protein the inventors also assumed that at least a portion of the AS CA IX molecules should be released into the culture medium. To investigate this possibility, the cells were cultivated under normoxia and hypoxia in the serum-free medium. After 24 h of incubation, one fourth of the culture medium was concentrated and analyzed by SDS-PAGE. Immunoblotting showed the presence of AS CA IX in the culture medium under both normoxia and hypoxia (FIG. 5B). Taken together, these data indicated that the AS is present in the intracellular as well as extracellular space, in contrast to FL CA IX, which is mostly confined to plasma membrane.

However, it was still possible that some AS molecules could be incorporated into heterooligomers with the FL CA IX. This assumption has been tested using the monoclonal antibody V/10, which normally binds to the intact domain of CA IX, but cannot recognize the AS variant (data not shown). This V/10 Mab was utilized for immunoprecipitation of the CA IX oligomers via its interaction with FL molecules. Components of the oligomers (including potentially incorporated AS molecules) were then resolved in reducing PAGE and visualized by immunoblotting using the peroxidase-labeled M75 antibody that reacts with both FL and AS forms. Under non-reducing conditions, the FL protein formed oligomers of about 153 K, whereas the AS CA IX variant was unable to do so and was also unable to enter into oligomers built by FL CA IX protein (FIG. 6; for details see Materials and Methods).

### Example 5

### Functional Properties of the Human AS CA IX

Expression of the FL CA IX in tumor cells is induced by hypoxia. Hypoxia also activates the catalytic performance of CA IX, which results in enhanced acidification of extracellular pH (Svastova et al, 2004). This acidification capacity can be abolished by overexpression of a dominant-negative mutant lacking the catalytic CA domain of CA IX (Svastova et al, 2004). Since the AS protein contains only incomplete CA domain, it was particularly important to analyze whether it is catalytically active and whether it is capable to disturb acidification mediated by the FL CA IX protein. Measurement of an enzyme activity was accomplished by stopped flow spectrometry using the recombinant bacterial GST-AS fusion protein containing the truncated CA domain compared to a GST-fused extracellular portion of the FL CA IX containing the complete CA domain [eg., SEQ ID NO: 9] and thereby forming GST-PGCA which contains both the PG and CA domains [aa 52-397 (SEQ ID NO: 83)]. The results revealed that the catalytic activity of the wild-type CA IX, K_{cat}(WT)=3.8×10⁵ s⁻¹, was reduced to a half in the splicing variant, K_{cat}(AS)=1.9×10⁵ s⁻¹. In addition, GST-AS protein showed considerably lower affinity for acetazolamine, a sulphonamide inhibitor of carbonic anhydrases: Kᵢ WT=14 nM versus Kᵢ AS=110 nM. Those data suggest that the splicing has compromised both, the enzyme activity of CA IX and its affinity to inhibitors.

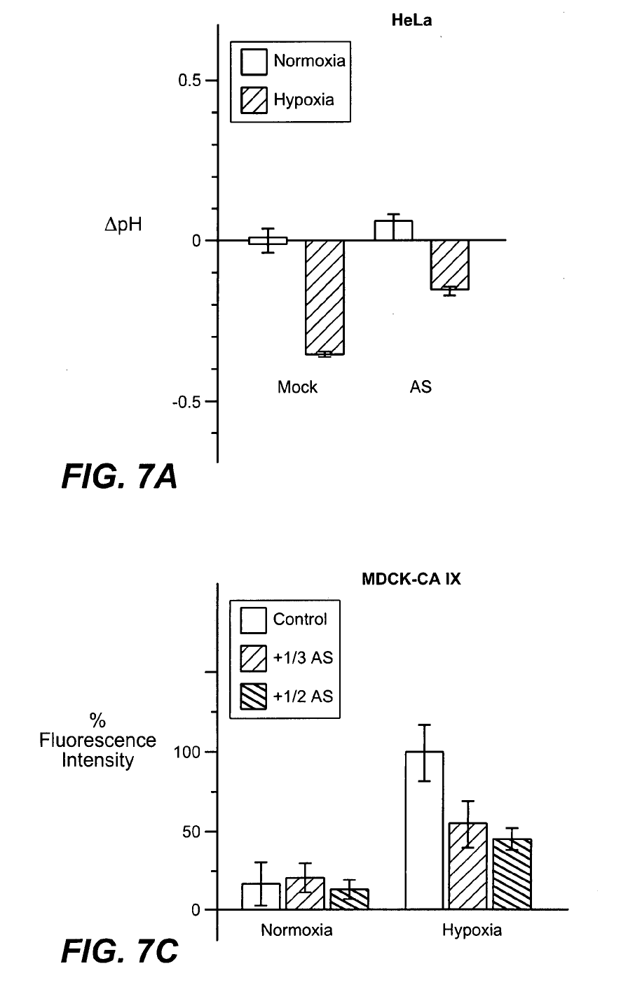

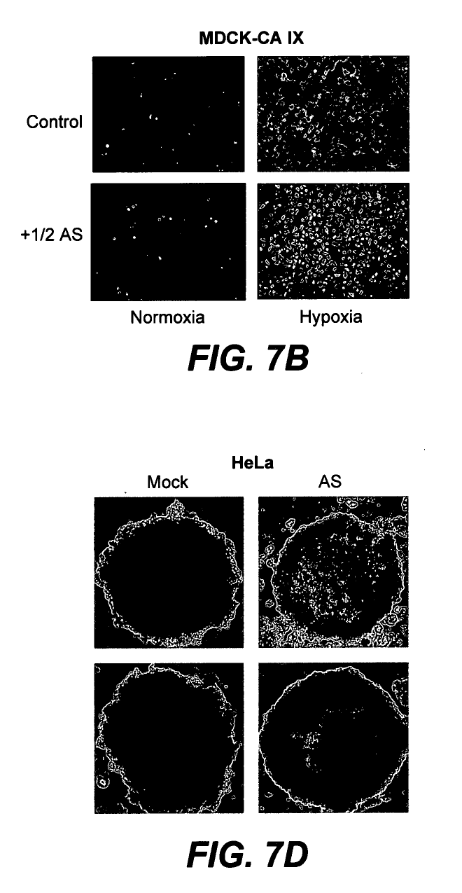

The inventors also wanted to learn whether the AS CA IX can modulate the capacity of the FL CA IX to acidify extracellular pH under hypoxic conditions. For that purpose the inventors analyzed the transfected HeLa-AS cells and the mock-transfected controls incubated for 48 h in 2% O₂ (hypoxia) and 21% O₂ (normoxia). Hypoxic incubation led to expected extracellular acidification in the control as well as in AS-transfected HeLa cells when compared to their normoxic counterparts (FIG. 7A). However, the medium was approximately 0.2 pH unit less acidified in the AS-overexpressing cells suggesting that the AS disturbed the activity of the wild-type CA IX protein.

Since the catalytic site of CA IX is exposed to extracellular space, the inventors tested a possible role of the extracellular fraction of AS. As described earlier, the activity of CA IX can be indirectly demonstrated using the fluorescein-labelled CA inhibitor (FITC-CAI) that binds only to hypoxia-activated CA IX whose catalytic site is accessible by the inhibitor (Svastova et al, 2004). Therefore, the inventors used an established model of CA IX-transfected MDCK cells that show CA IX-mediated extracellular acidification when treated by hypoxia and accumulate FITC-CAI in hypoxia but not in normoxia. Here the inventors analyzed the accumulation of FITC-CAI in MDCK-CA IX cells in the presence and absence of culture medium from the AS-secreting MDCK-AS transfectants. As shown on FIG. 7B, incubation of MDCK-CA IX cells in the fresh medium mixed with the AS-containing conditioned medium resulted in visibly reduced accumulation of FITC-CA IX supporting the idea that the extracellular AS diminished the binding of the inhibitor. This experiment has been repeated with one half as well as one third of the AS-containing conditioned medium. The acquired images were analyzed to determine the differences in intensity of fluorescence. The results clearly proved that the extracellular fraction of AS reduced FITC-CAI accumulation approximately to a half (FIG. 7C).

To see whether the effect of the AS variant on the functioning of the FL CA IX could have biological consequences, the inventors analyzed the growth parameters of the HeLa-AS transfectants compared to the mock-transfected controls. No significant differences were observed between these two cell types upon their short-term (72 h) growth in adherent culture independently of normoxic or hypoxic conditions (data not shown). Therefore, the inventors also produced HeLa cell spheroids grown for 14 days and compared the mass and shape of the spheroids generated from the HeLa-AS cells and the control HeLa cells, respectively. The HeLa-AS spheroids were less compact and lacked the central region, which usually contains the cells that suffer from low oxygen and acidic pH (FIG. 7D). The appearance of these HeLa-AS spheroids suggested that the effect of AS, which leads to reduced capacity of the FL CA IX to modulate pH, could influence the capability of cells to survive these microenvironmental stresses.

Altogether, our results showed that the AS CA IX is differently regulated, abnormally localized and functionally disabled when compared to FL CA IX.

### Discussion

Deregulation of alternative splicing is a well-recognized phenomenon particularly in cancer (Venables et al, 2006). There are numerous examples of alternatively spliced genes whose products are causally involved in tumor progression, such as CD44, HIF-α, VEGF, osteopontin and many others (Wong et al, 2003, Gothie et al, 2000, Robinson et al, 2001, He et al, 2006). In some cases, the splice form that is rare in normal tissues can become common in tumors, while the alternative splice form present in normal tissues can remain constant (Roy et al, 2005).

Alternative splicing variant of the human CA IX identified in this study can be classified to this category, although it is difficult to make a clear-cut conclusion, since the expression pattern of the full-length CA IX is quite particular. The FL CA IX is abundant in very few normal tissues including the stomach and small intestine, which at the same time express low level of the alternative splicing variant. In gastric carcinomas, expression of the FL CA IX decreases, but the level of AS is similar as in the normal stomach. On the other hand, expression of the full-length CA IX is absent or very low in the normal colon and rectum (and also in additional normal tissues not analyzed in this study) and significantly increases in corresponding tumors (Saarnio et al, 1998). However, the AS variant shows a steady expression level in both normal tissues and colorectal carcinomas. These data strongly suggest that its expression is not linked to tumor phenotype. Moreover, in contrast to the FL CA IX whose levels are induced in the cells growing in crowded culture and exposed to low oxygen, the AS variant is not principally dependent on hypoxia and cell density.

Relatively low, but constitutive expression of AS is of considerable importance for clinical studies using CA9 transcription as a marker of hypoxic tumors for potential prognostic or predictive purposes. Because of the presence of AS in the absence of FL CA9 transcript in the normal and/or non-hypoxic tissues, primers or probes designed for detection of the regions that are not affected by the splicing cannot differentiate between the two forms of CA9 mRNA and thus might give false-positive results, which could influence the real clinical value of the hypoxia-induced FL CA9.

Noteworthy, 5′ RACE analysis of the AS mRNA compared to the FL transcript has generated the products of identical length supporting the conclusion that both variants are produced from the same promoter (data not shown). This fact might suggest a differential cooperation of the transcriptional apparatus with the components of the splicing machinery in the processing of the CA9 transcript depending on the physiological circumstances. Indeed, there are several examples of the splicing events regulated by hypoxia such as those related to hTERT, TrkA and XBP1 (Anderson et al, 2006, Taconelli et al, 2004, Romero-Ramirez et al, 2004). In the case of hTERT it has been demonstrated that the transcriptional complex containing RNA polymerase 11, TFIIB, HIF and co activators recruits at the promoter under hypoxia and remains associated with the gene as long as transcription proceeds. This induces switch in the splice pattern in favour of an active form of the enzyme (Anderson et al, 2006). It is quite conceivable that a similar mechanism might operate during the transcription of the CA9 gene.

The AS variant of the human CA9 mRNA results from deletion of exons 8 plus 9 and is translated to truncated protein which does not contain the transmembrane region, intracellular tail and C-terminal part of the catalytic domain. Removal of the TM and IC regions is apparently responsible for the altered localization of this AS variant, which predominantly occupies intracellular space and is also released to extracellular medium. This is contrasting with the FL CA IX protein, which is an integral plasma membrane protein. Such inappropriate localization linked with a partial deletion of the catalytic domain can be expected to compromise the protein functionality. Indeed, GST-AS shows only half of the enzyme activity of the corresponding GST-PG+CA protein containing the complete catalytic domain. However, it is very difficult to translate this finding directly into local cellular context, where CA IX interacts with bicarbonate transporters and contributes to pH regulation across the plasma membrane under hypoxic conditions (Morgan et al, 2007, Svastova et al, 2004, Swietach et al, 2007). Firstly, the activity measurements were performed with the proteins produced in bacteria in a setting free of any subcellular structures, protein-protein interactions, ion fluxes and microenvironmental influences which certainly play a role in modulating the catalytic performance of CA IX. Secondly, the catalytic activities of different carbonic anhydrase isoenzymes vary roughly within two orders of magnitude, with the highly active isoforms showing from 20- to only 3-times higher activity than the isoenzymes that are considered moderate (Pastorekova et al, 2004). So it is not possible to preclude whether the half-reduced activity would be sufficient for the physiological function of CA IX. Anyhow, this question is probably not critical, since the AS variant is not properly localized at the plasma membrane and is unable to form oligomers, which are very important constraints for the CA IX protein functioning.

However, reduced extracellular acidification observed in the culture of hypoxic HeLa-AS cells that constitutively overexpress the AS form of CA IX clearly indicates that it interferes with the function of endogenous, hypoxia-induced FL protein. Although the mechanism is not clear at present, based on the decreased accumulation of CA inhibitor in the hypoxic MDCK-CA IX cells treated with the AS variant, one can propose that AS competes with the FL CA IX for an interaction with the cell surface components of the bicarbonate transport metabolon. Moreover, overexpression of AS considerably affects the capacity of HeLa cells to form compact spheroids, which are often used as a 3D model that mimics tumor mass with corresponding intratumoral microenvironment. Many studies well document gradients of oxygen partial pressure, pH, nutrients and metabolites across the spheroids whose core regions show clear analogy with the hypoxic areas of solid tumors that are characterized by more acidic microenvironment (Alvarez-Perez et al, 2005). It has been shown elsewhere that the plasma membrane staining of the FL CA IX is significantly increased in the innermost cells of multicellular spheroids generated from SiHa and HeLa cervical carcinoma cells (Olive et al, 2001, Chrastina et al, 2003). These data indicate that FL CA IX is present exactly in the areas where the cells need increased protection and/or adaptation to harmful effects of the hypoxic stress and acidic microenvironment in order to survive. The FL CA IX acts here via bicarbonate-mediated buffering of intracellular pH (Swietach et al, 2007). The AS variant that partially perturbs this pH regulation, obviously does not permit the adaptation to acidic intra-spheroid pH, leading to elimination of the most stressed central cells from the core of spheroids. This idea is consistent with the findings that the catalytic activity of CA IX is regulated by hypoxia and suggests that the capacity of CA IX to modulate pH is vital for the survival of hypoxic tumor cells. The latter suggestion has been indirectly supported also by RNAi experiments by Robertson et al (2004).

Although the naturally produced AS variant is expressed at low level, there are physiological situations and cell types that only weakly induce FL CA IX. For example tumor cells localized at shorter distances from functional blood-supplying vessels are exposed to mild hypoxia and may express comparable levels of FL and AS allowing thus for dominant-negative down-modulation of CA IX activity. Such weakly hypoxic cells are presumably not exposed to severe acidosis and therefore may not benefit from full performance of this pH-control mechanism. Similar explanation can be applied also to normal tissues suffering from mild ischemia. This idea finds support in the recent as well as previous data showing that some tumor cell lines, dense normoxic cells (affected by weak pericellular hypoxia) and some early stage less-hypoxic tumors express just low levels of FL CA IX. In conclusion, the inventors propose that the AS variant functions as a modulator of the FL CA IX under circumstances when both proteins are co-expressed. The low but constitutive expression of the alternative splicing variant is of considerable importance for clinical studies based on CA9 transcription as a marker of hypoxic tumors for potential prognostic or predictive purposes. Because of the presence of AS in the absence of FL CA9 transcript in normal and/or non-hypoxic tissues, primers or probes designed for detection of the regions that are not affected by the splicing cannot differentiate between the two forms of CA9 mRNA and thus might give false-positive results, which could influence the real clinical value of hypoxia-induced FL CA9. This could, in fact, happen in several studies that have been published so far [e.g. McKiernan et al., Cancer, 86(3): 492497 (1999); Span et al., Br J Cancer, 89(2): 271-276 (2003); Simi et al., Lung Cancer, 52(1): 59-66 (2006); Greiner et al., Blood. 108(13): 4109-4117 (2006)]. For this reason, design of correct primers and probes for microarray chips and RT-PCR should be made with precaution and should take into account the AS form of MN/CA IX.

### Budapest Treaty Deposits

The materials listed below were deposited with the American Type Culture Collection (ATCC) now at 10810 University Blvd., Manassas, Va. 20110-2209 (USA). The deposits were made under the provisions of the Budapest Treaty on the International Recognition of Deposited Microorganisms for the Purposes of Patent Procedure and Regulations there under (Budapest Treaty). Maintenance of a viable culture is assured for thirty years from the date of deposit. The hybridomas and plasmids will be made available by the ATCC under the terms of the Budapest Treaty, and subject to an agreement between the Applicants and the ATCC which assures unrestricted availability of the deposited hybridomas and plasmids to the public upon the granting of patent from the instant application. Availability of the deposited strain is not to be construed as a license to practice the invention in contravention of the rights granted under the authority of any Government in accordance with its patent laws.

| | Deposit Date | ATCC # |
|---|---|---|
| Hybridoma | | |
| VU-M75 | Sep. 17, 1992 | HB 11128 |
| MN 12.2.2 | Jun. 9, 1994 | HB 11647 |
| Plasmid | | |
| A4a | Jun. 6, 1995 | 97199 |
| XE1 | Jun. 6, 1995 | 97200 |
| XE3 | Jun. 6, 1995 | 97198 |

Similarly, the hybridoma cell line V/10-VU which produces the V/10 monoclonal antibodies was deposited on Feb. 19, 2003 under the Budapest Treaty at the International Depository Authority (IDA) of the Belgian Coordinated Collections of Microorganisms (BCCM) at the Laboratorium voor Moleculaire Biologie-Plasmidencollectie (LMBP) at the Universeit Gent, K. L. Ledeganckstraat 35, B-9000 Gent, Belgium [BCCM/LMBP] under the Accession No. 6009CB.

The description of the foregoing embodiments of the invention have been presented for purposes of illustration and description. They are not intended to be exhaustive or to limit the invention to the precise form disclosed, and obviously many modifications and variations are possible in light of the above teachings. The embodiments were chosen and described in order to explain the principles of the invention and its practical application to enable thereby others skilled in the art to utilize the invention in various embodiments and with various modifications as are suited to the particular use contemplated.

All references cited herein are hereby incorporated by reference.

1. A diagnostic and/or prognostic method for a preneoplastic/neoplastic disease associated with abnormal MN/CA IX expression in a mammal, comprising differentiating between full-length [FL] and alternatively-spliced [AS] MN/CA9 mRNA or MN/CA IX expression.

2. The method of claim 1, comprising the use of one or more probes and/or primers to detect or detect and quantitate FL and/or AS MN/CA9 mRNA expression.

3. The method of claim 2, comprising the use of:
(a) probes and/or primers to detect full-length [FL] MN/CA9 mRNA but not alternatively-spliced [AS] MN/CA9 mRNA;
(b) probes and/or primers to detect AS MN/CA9 mRNA but not FL MN/CA9 mRNA; and/or
(c) probes and/or primers to detect both FL and AS MN/CA9 mRNA.

4. The method of claim 2, wherein said mammal is a human, and wherein said one or more probes and/or primers is/are selected from the group consisting of SEQ ID NOS: 97-101 and nucleic acid sequences that are at least 80% homologous to SEQ ID NOS: 97-101.

5. The method of claim 2, comprising the use of a nucleic acid amplification method.

6. The method of claim 5, wherein said nucleic acid amplification method comprises the use of PCR, RT-PCR, real-time PCR or quantitative real-time RT-PCR.

7. The method of claim 2, comprising the use of a microarray chip that comprises a probe that binds to full-length [FL] MN/CA9 mRNA but not to alternatively-spliced [AS] MN/CA9 mRNA, and/or a probe that binds to AS MN/CA9 mRNA but not FL MN/CA9 mRNA.

8. The method of claim 2, further comprising determining the ratio of FL:AS MN/CA9 mRNA.

9. The method of claim 2, wherein said AS MN/CA9 mRNA expression indicates normal MN/CA9 gene expression, and said FL MN/CA9 mRNA expression indicates abnormal MN/CA9 gene expression.

10. The method of claim 2, wherein said AS MN/CA9 mRNA expression indicates normoxic MN/CA9 gene expression, and said FL MN/CA9 mRNA expression indicates hypoxic MN/CA9 gene expression.

11. The method of claim 1, comprising the use of one or more antibodies to differentiate between FL and AS MN/CA IX expression in a preneoplastic/neoplastic tissue.

12. The method of claim 11, comprising detecting or detecting and quantitating AS MN/CA IX in said tissue.

13. The method of claim 12, further comprising determining the ratio of FL MN/CA IX levels to AS MN/CA IX levels in said tissue.

14. The method of claim 13, wherein said ratio indicates presence or degree of hypoxia in said tissue.

15. The method of claim 11, comprising detecting or detecting and quantitating FL MN/CA IX and AS MN/CA IX in a vertebrate tissue, comprising the steps of:
(a) contacting a sample of said vertebrate tissue synchronously or sequentially with at least two antibodies, at least two antigen-binding antibody fragments, or a mixture of antibodies and antigen-binding antibody fragments, wherein at least one antibody/antibody fragment specifically binds to FL MN/CA IX protein but not to AS MN/CA IX protein, and wherein at least one other antibody/antibody fragment specifically binds to both FL and AS MN/CA IX;
(b) detecting and quantifying the binding of said antibodies/antibody fragments in said sample; and
(c) comparing the binding of said differentially binding antibodies/antibody fragments to determine the relative levels of FL MN/CA IX and AS MN/CA IX.

16. The method of claim 15, wherein the antibody/antibody fragment, or antibodies/antibody fragments, that specifically bind(s) to FL MN/CA IX but not to AS MN/CA IX is/are specific for the carbonic anhydrase (CA) domain of MN/CA IX; and wherein the antibody/antibody fragment, or antibodies/antibody fragments, that specifically bind(s) both FL MN/CA IX and AS MN/CA IX is/are specific for the proteoglycan-like (PG) domain of MN/CA IX.

17. The method of claim 16, wherein said antibody specific for the CA domain of MN/CA IX is the V/10 monoclonal antibody which is produced by the hybridoma VU-V/10, deposited at BCCM™/LMBP in Ghent, Belgium under Accession No. LMBP 6009CB; and wherein the antibody specific for the PG domain of MN/CA IX is the M75 monoclonal antibody which is produced by the hybridoma VU-M75 deposited at the American Type Culture Collection (ATCC) under the ATCC designation No. HB 11128.

18. A diagnostic and/or prognostic method for a preneoplastic/neoplastic disease associated with abnormal MN/CA IX expression in a vertebrate, comprising detecting or detecting and quantitating full-length [FL] MN/CA IX protein but not alternatively-spliced [AS] MN/CA IX protein in a vertebrate tissue, comprising the steps of:
(a) contacting a sample of said vertebrate tissue with an antibody or antibody fragment, wherein said antibody or antibody fragment specifically binds to FL MN/CA IX but not to AS MN/CA IX; and
(b) detecting and quantifying binding of said antibody/antibody fragment in said sample.

19-20. (canceled)

21. A diagnostic and/or prognostic method for a preneoplastic/neoplastic disease associated with abnormal MN/CA IX expression in a mammal, comprising detecting or detecting and quantitating full-length [FL] MN/CA9 mRNA but not alternatively-spliced [AS] MN/CA9 mRNA in a mammalian preneoplastic/neoplastic sample, comprising contacting mRNA from said sample with a primer or a probe that specifically binds to FL MN/CA9 mRNA but not to AS MN/CA9 mRNA.

22-29. (canceled)

30. A pair of probes and/or primers used to differentiate between alternatively-spliced [AS] MN/CA9 mRNA and full-length [FL] MN/CA9 mRNA expression in a mammal.

31-35. (canceled)

36. An isolated nucleic acid encoding a mammalian alternatively-spliced [AS] MN/CA IX, wherein said AS MN/CA IX has a molecular weight of from about 43 to about 48 kilodaltons.

37-43. (canceled)

44. An antibody or antigen-binding antibody fragment that binds specifically to the AS MN/CA IX of claim 36, but does not bind specifically to FL MN/CA IX.

45. An antibody or antigen binding antibody fragment that binds specifically to the AS MN/CA IX of claim 36, but does not bind specifically to soluble MN/CA IX (s-CA IX).

46. A method for treating preneoplastic/neoplastic disease in a mammal, wherein said disease is associated with abnormal expression of MN/CA IX, the method comprising administering to said mammal a therapeutically effective amount of a composition comprising an agent that increases levels of alternatively-spliced [AS] MN/CA IX relative to levels of full-length [FL] MN/CA IX.

47-50. (canceled)

51. An oligonucleotide that increases levels of alternatively-spliced [AS] MN/CA IX relative to levels of full-length [FL] MN/CA IX, wherein said oligonucleotide is used in treatment of a preneoplastic/neoplastic disease associated with abnormal MN/CA IX expression.

52-54. (canceled)

55. An in vitro method of identifying agents capable of modulating levels of alternatively-spliced [AS] MN/CA IX, comprising contacting cells expressing AS MN/CA IX with an agent suspected of modulating the level of said AS MN/CA IX in the cells, and detecting and quantitating changes in levels of said AS MN/CA IX.
